SEQUENCE LISTING (45 sequences)

SEQ ID NO: 1 - protein, 107 aa, Mus musculus
QIVLSQSPAILSASPGEKVTMTCRASSSVSYMHWYQQKPGSSPKPWIYAPSNLASGVPARFSGSGSGTSYSLTISRVEAEDAATYYCQQWSFNPPTFGAGTKLELKR

SEQ ID NO: 2 - protein, 107 aa, Artificial sequence
  sequence is synthesized
DIQMTQSPSSLSASVGDRVTITCRASSSVSYMHWYQQKPGKAPKPLIYAPSNLASGVPSRFSGSGSGTDFTLTISSLQPEDFATYYCQQWSFNPPTFGQGTKVEIKR

SEQ ID NO: 3 - protein, 108 aa, Homo sapiens
DIQMTQSPSSLSASVGDRVTITCRASQSISNYLAWYQQKPGKAPKLLTYAASSLESGVPSRFSGSGSGTDFTLTISSLQPEDFATYYCQQYNSLPWTFGQGTKVEIKR

SEQ ID NO: 4 - protein, 10 aa, Mus musculus
RASSSVSYMH

SEQ ID NO: 5 - protein, 7 aa, Mus musculus
APSNLAS

SEQ ID NO: 6 - protein, 9 aa, Mus musculus
QQWSFNPPT

SEQ ID NO: 7 - protein, 122 aa, Mus musculus
QAYLQQSGAELVRPGASVKMSCKASGYTFTSYNMHWVKQTPRQGLEWIGAIYPGNGDTSYNQKFKGKATLTVDKSSSTAYMQLSSLTSEDSAVYFCARVVYYSNSYWYFDVWGTGTTVTVSS

SEQ ID NO: 8 - protein, 122 aa, Artificial sequence [sequence omitted]

SEQ ID NO: 9 - protein, 119 aa, Homo sapiens
EVQLVESGGGLVQPGGSLRLSCAASGFTFSSYAMSWVRQAPGKGLEWVAVISGDGGSTYYADSVKGRFTISRDNSKNTLYLQMNSLRAEDTAVYYCARGRVGYSLYDYWGQGTLVTVSS

SEQ ID NO: 10 - protein, 10 aa, Mus musculus
GYTFTSYNMH

SEQ ID NO: 11 - protein, 17 aa, Mus musculus
AIYPGNGDTSYNQKFKG

SEQ ID NO: 12 - protein, 13 aa, Mus musculus
VVYYSNSYWYFDV

SEQ ID NO: 13 - DNA, 5679 bp, Artificial Sequence
  sequence is synthesized
gaattcaacttctccatactttggataaggaaatacagacatgaaaaatctcattgctgagttgttatttaagcttgcccaaaaagaagaagagtcgaatgaactgtgtgcgcaggtagaagctttggagattatcgtcactgcaatgcttcgcaatatggcgcaaaatgaccaacagcggttgattgatcaggtagagggggcgctgtacgaggtaaagcccgatgccagcattcctgacgacgatacggagctgctgcgcgattacgtaaagaagttattgaagcatcctcgtcagtaaaaagttaatcttttcaacagctgtcataaagttgtcacggccgagacttatagtcgctttgtttttattttttaatgtatttgtaactagaattcgagctcggtacccggggatcctctagaggttgaggtgattttatgaaaaagaatatcgcatttcttcttgcatctatgttcgttttttctattgctacaaacgcgtacgctgatatccagatgacccagtccccgagctccctgtccgcctctgtgggcgatagggtcaccatcacctgcagagccagtcagagcgtgtcgactagctcttatagctatatgcactggtatcaacagaaaccaggaaaagctccgaaactactgatttactatgctagcaacctcgagtctggagtcccttctcgcttctctggatccggttctgggacggatttcactctgaccatcagcagtctgcagccagaagacttcgcaacttattactgtcaacactcttggggtattccgcgcacatttggacagggtaccaaggtggagatcaaacgaactgtggctgcaccatctgtcttcatcttcccgccatctgatgagcagttgaaatctggaactgcttctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttaagctgatcctctacgccggacgcatcgtggccctagtacgcaagttcacgtaaaaagggtatctagaggttgaggtgattttatgaaaaagaatatcgcatttcttcttgcatctatgttcgttttttctattgctacaaacgcgtacgctgaggttcagctggtggagtctggcggtggcctggtgcagccagggggctcactccgtttgtcctgtgcagcttctggctacaccttcaccgaatatatcatccactgggtccgtcaggccccgggtaagggcctggaatgggttgcatcgattaatcctgactacgacatcacgaactataaccagcgcttcaagggccgtttcactataagtcgcgacgattccaaaaacacattatacctgcagatgaacagcctgcgtgctgaggacactgccgtctattattgtgctcgatggatcagcgatttcttcgactactggggtcaaggaaccctggtcaccgtctcctcggcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgaccaccgcatgcaccagtatcgtccattccgacagcatcgccagtcactatggcgtgctgctagcgccgccctataccttgtctgcctccccgcgttgcgtcgcggtgcatggagccgggccacctcgacctgaatggaagccggcggcacctcgctaacggattcaccactccaagaattggagccaatcaattcttgcggagaactgtgaatgcgcaaaccaacccttggcagaacatatccatcgcgtccgccatctccagcagccgcacgcggcgcatctcgggcagcgttgggtcctggccacgggtgcgcatgatcgtgctcctgtcgttgaggacccggctaggctggcggggttgccttactggttagcagaatgaatcaccgatacgcgagcgaacgtgaagcgactgctgctgcaaaacgtctgcgacctgagcaacaacatgaatggtcttcggtttccgtgtttcgtaaagtctggaaacgcggaagtcagcgccctgcaccattatgttccggatctgcatcgcaggatgctgctggctaccctgtggaacacctacatctgtattaacgaagcgctggcattgaccctgagtgatttttctctggtcccgccgcatccataccgccagttgtttaccctcacaacgttccagtaaccgggcatgttcatcatcagtaacccgtatcgtgagcatcctctctcgtttcatcggtatcattacccccatgaacagaaattcccccttacacggaggcatcaagtgaccaaacaggaaaaaaccgcccttaacatggcccgctttatcagaagccagacattaacgcttctggagaaactcaacgagctggacgcggatgaacaggcagacatctgtgaatcgcttcacgaccacgctgatgagctttaccgcagcatccggaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggctatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtccgcatcctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaa

SEQ ID NO: 14 - DNA, 5679 bp, Artificial sequence
  sequence is synthesized
ttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcaggatgcggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatagccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttccggatgctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcacttgatgcctccgtgtaagggggaatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggccaggacccaacgctgcccgagatgcgccgcgtgcggctgctggagatggcggacgcgatggatatgttctgccaagggttggtttgcgcattcacagttctccgcaagaattgattggctccaattcttggagtggtgaatccgttagcgaggtgccgccggcttccattcaggtcgaggtggcccggctccatgcaccgcgacgcaacgcggggaggcagacaaggtatagggcggcgctagcagcacgccatagtgactggcgatgctgtcggaatggacgatactggtgcatgcggtggtcatgtgtgagttttgtcacaagatttgggctcaactttcttgtccaccttggtgttgctgggcttgtgattcacgttgcagatgtaggtctgggtgcccaagctgctggagggcacggtcaccacgctgctgagggagtagagtcctgaggactgtaggacagccgggaaggtgtgcacgccgctggtcagggcgcctgagttccacgacaccgtcaccggttcggggaagtagtccttgaccaggcagcccagggccgctgtgcccccagaggtgctcttggaggagggtgccagggggaagaccgatgggcccttggtggaggccgaggagacggtgaccagggttccttgaccccagtagtcgaagaaatcgctgatccatcgagcacaataatagacggcagtgtcctcagcacgcaggctgttcatctgcaggtataatgtgtttttggaatcgtcgcgacttatagtgaaacggcccttgaagcgctggttatagttcgtgatgtcgtagtcaggattaatcgatgcaacccattccaggcccttacccggggcctgacggacccagtggatgatatattcggtgaaggtgtagccagaagctgcacaggacaaacggagtgagccccctggctgcaccaggccaccgccagactccaccagctgaacctcagcgtacgcgtttgtagcaatagaaaaaacgaacatagatgcaagaagaaatgcgatattctttttcataaaatcacctcaacctctagataccctttttacgtgaacttgcgtactagggccacgatgcgtccggcgtagaggatcagcttaacactctcccctgttgaagctctttgtgacgggcgagctcaggccctgatgggtgacttcgcaggcgtagactttgtgtttctcgtagtctgctttgctcagcgtcagggtgctgctgaggctgtaggtgctgtccttgctgtcctgctctgtgacactctcctgggagttacccgattggagggcgttatccaccttccactgtactttggcctctctgggatagaagttattcagcaggcacacaacagaagcagttccagatttcaactgctcatcagatggcgggaagatgaagacagatggtgcagccacagttcgtttgatctccaccttggtaccctgtccaaatgtgcgcggaataccccaagagtgttgacagtaataagttgcgaagtcttctggctgcagactgctgatggtcagagtgaaatccgtcccagaaccggatccagagaagcgagaagggactccagactcgaggttgctagcatagtaaatcagtagtttcggagcttttcctggtttctgttgataccagtgcatatagctataagagctagtcgacacgctctgactggctctgcaggtgatggtgaccctatcgcccacagaggcggacagggagctcggggactgggtcatctggatatcagcgtacgcgtttgtagcaatagaaaaaacgaacatagatgcaagaagaaatgcgatattctttttcataaaatcacctcaacctctagaggatccccgggtaccgagctcgaattctagttacaaatacattaaaaaataaaaacaaagcgactataagtctcggccgtgacaactttatgacagctgttgaaaagattaactttttactgacgaggatgcttcaataacttctttacgtaatcgcgcagcagctccgtatcgtcgtcaggaatgctggcatcgggctttacctcgtacagcgccccctctacctgatcaatcaaccgctgttggtcattttgcgccatattgcgaagcattgcagtgacgataatctccaaagcttctacctgcgcacacagttcattcgactcttcttctttttgggcaagcttaaataacaactcagcaatgagatttttcatgtctgtatttccttatccaaagtatggagaagttgaattc

SEQ ID NO: 15 - protein, 241 aa, Artificial sequence
  sequence is synthesized
MKKNIAFLLASMFVFSIATNAYADIQMTQSPSSLSASVGDRVTITCRASQSVSTSSYSYMHWYQQKPGKAPKLLIYYASNLESGVPSRFSGSGSGTDFTLTISSLQPEDFATYYCQHSWGIPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 16 - protein, 248 aa, Artificial sequence
  sequence is synthesized
MKKNIAFLLASMFVFSIATNAYAEVQLVESGGGLVQPGGSLRLSCAASGYTFTEYIIHWVRQAPGKGLEWVASINPDYDITNYNQRFKGRFTISRDDSKNTLYLQMNSLRAEDTAVYYCARWISDFFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHT

SEQ ID NO: 17 - DNA, 5678 bp, Artificial Sequence
  sequence is synthesized
gaattcaacttctccatactttggataaggaaatacagacatgaaaaatctcattgctgagttgttatttaagcttgcccaaaaagaagaagagtcgaatgaactgtgtgcgcaggtagaagctttggagattatcgtcactgcaatgcttcgcaatatggcgcaaaatgaccaacagcggttgattgatcaggtagagggggcgctgtacgaggtaaagcccgatgccagcattcctgacgacgatacggagctgctgcgcgattacgtaaagaagttattgaagcatcctcgtcagtaaaaagttaatcttttcaacagctgtcataaagttgtcacggccgagacttatagtcgctttgtttttattttttaatgtatttgtaactagaattcgagctcggtacccggggatcctctagaggttgaggtgatttatgaaaaagaatatcgcatttcttcttgcatctatgttcgttttttctattgctacaaacgcgtacgctcagatagtactgtcccagtccccggctatcctgtccgcctctcctggcgagaaggtcactatgacctgcagagccagctcttctgtgagctatatgcattggtatcaacagaaaccaggaagctctccgaaaccatggatttacgctccatcgaacctcgcgtctggagtccctgcgcgcttctctggatccggttctgggactagttactctctgaccatcagcagagtggaggcagaagacgccgcaacttattactgtcaacagtggagcttcaatccgcccacatttggagccggcaccaagctggagctcaaacgaactgtggctgcaccatctgtcttcatcttcccgccatctgatgagcagttgaaatctggaactgcttctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttaagctgatcctctacgccggacgcatcgtggccctagtacgcaagttcacgtaaaaagggtatctagaggttgaggtgattttatgaaaaagaatatcgcatttcttcttgcatctatgttcgttttttctattgctacaaacgcgtacgctcaggcttatctgcagcagtctggcgccgagctggtgcggccaggagctagcgtcaagatgtcctgtaaagcttctggctacaccttcaccagctataacatgcattgggtcaagcagacaccgaggcaaggcctggaatggattggagcgatctatcctggcaacggcgacacgagctataaccagaagttcaagggcaaggccactctgactgtggacaagtccagcagtactgcctacatgcaactgagcagcctgacttctgaggacagcgctgtctacttttgtgctcgcgtggtctactatagcaacagctactggtacttcgacgtctggggtaccggaaccacagtcaccgtctcctcggcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgaccaccgcatgcaccagtatcgtccattccgacagcatcgccagtcactatggcgtgctgctagcgccgccctataccttgtctgcctccccgcgttgcgtcgcggtgcatggagccgggccacctcgacctgaatggaagccggcggcacctcgctaacggattcaccactccaagaattggagccaatcaattcttgcggagaactgtgaatgcgcaaaccaacccttggcagaacatatccatcgcgtccgccatctccagcagccgcacgcggcgcatctcgggcagcgttgggtcctggccacgggtgcgcatgatcgtgctcctgtcgttgaggacccggctaggctggcggggttgccttactggttagcagaatgaatcaccgatacgcgagcgaacgtgaagcgactgctgctgcaaaacgtctgcgacctgagcaacaacatgaatggtcttcggtttccgtgtttcgtaaagtctggaaacgcggaagtcagcgccctgcaccattatgttccggatctgcatcgcaggatgctgctggctaccctgtggaacacctacatctgtattaacgaagcgctggcattgaccctgagtgatttttctctggtcccgccgcatccataccgccagttgtttaccctcacaacgttccagtaaccgggcatgttcatcatcagtaacccgtatcgtgagcatcctctctcgtttcatcggtatcattacccccatgaacagaaattcccccttacacggaggcatcaagtgaccaaacaggaaaaaaccgcccttaacatggcccgctttatcagaagccagacattaacgcttctggagaaactcaacgagctggacgcggatgaacaggcagacatctgtgaatcgcttcacgaccacgctgatgagctttaccgcagcatccggaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggctatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtccgcatcctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaa

SEQ ID NO: 18 - DNA, 5678 bp, Artificial sequence
  sequence is synthesized
ttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcaggatgcggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatagccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttccggatgctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcacttgatgcctccgtgtaagggggaatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggccaggacccaacgctgcccgagatgcgccgcgtgcggctgctggagatggcggacgcgatggatatgttctgccaagggttggtttgcgcattcacagttctccgcaagaattgattggctccaattcttggagtggtgaatccgttagcgaggtgccgccggcttccattcaggtcgaggtggcccggctccatgcaccgcgacgcaacgcggggaggcagacaaggtatagggcggcgctagcagcacgccatagtgactggcgatgctgtcggaatggacgatactggtgcatgcggtggtcatgtgtgagttttgtcacaagatttgggctcaactttcttgtccaccttggtgttgctgggcttgtgattcacgttgcagatgtaggtctgggtgcccaagctgctggagggcacggtcaccacgctgctgagggagtagagtcctgaggactgtaggacagccgggaaggtgtgcacgccgctggtcagggcgcctgagttccacgacaccgtcaccggttcggggaagtagtccttgaccaggcagcccagggccgctgtgcccccagaggtgctcttggaggagggtgccagggggaagaccgatgggcccttggtggaggccgaggagacggtgactgtggttccggtaccccagacgtcgaagtaccagtagctgttgctatagtagaccacgcgagcacaaaagtagacagcgctgtcctcagaagtcaggctgctcagttgcatgtaggcagtactgctggacttgtccacagtcagagtggccttgcccttgaacttctggttatagctcgtgtcgccgttgccaggatagatcgctccaatccattccaggccttgcctcggtgtctgcttgacccaatgcatgttatagctggtgaaggtgtagccagaagctttacaggacatcttgacgctagctcctggccgcaccagctcggcgccagactgctgcagataagcctgagcgtacgcgtttgtagcaatagaaaaaacgaacatagatgcaagaagaaatgcgatattctttttcataaaatcacctcaacctctagataccctttttacgtgaacttgcgtactagggccacgatgcgtccggcgtagaggatcagcttaacactctcccctgttgaagctctttgtgacgggcgagctcaggccctgatgggtgacttcgcaggcgtagactttgtgtttctcgtagtctgctttgctcagcgtcagggtgctgctgaggctgtaggtgctgtccttgctgtcctgctctgtgacactctcctgggagttacccgattggagggcgttatccaccttccactgtactttggcctctctgggatagaagttattcagcaggcacacaacagaagcagttccagatttcaactgctcatcagatggcgggaagatgaagacagatggtgcagccacagttcgtttgagctccagcttggtgccggctccaaatgtgggcggattgaagctccactgttgacagtaataagttgcggcgtcttctgcctccactctgctgatggtcagagagtaactagtcccagaaccggatccagagaagcgcgcagggactccagacgcgaggttcgatggagcgtaaatccatggtttcggagagcttcctggtttctgttgataccaatgcatatagctcacagaagagctggctctgcaggtcatagtgaccttctcgccaggagaggcggacaggatagccggggactgggacagtactatctgagcgtacgcgtttgtagcaatagaaaaaacgaacatagatgcaagaagaaatgcgatattctttttcataaatcacctcaacctctagaggatccccgggtaccgagctcgaattctagttacaaatacattaaaaaataaaaacaaagcgactataagtctcggccgtgacaactttatgacagctgttgaaaagattaactttttactgacgaggatgcttcaataacttctttacgtaatcgcgcagcagctccgtatcgtcgtcaggaatgctggcatcgggctttacctcgtacagcgccccctctacctgatcaatcaaccgctgttggtcattttgcgccatattgcgaagcattgcagtgacgataatctccaaagcttctacctgcgcacacagttcattcgactcttcttctttttgggcaagcttaaataacaactcagcaatgagatttttcatgtctgtatttccttatccaaagtatggagaagttgaattc

SEQ ID NO: 19 - protein, 236 aa, Artificial sequence
  sequence is synthesized
MKKNIAFLLASMFVFSIATNAYAQIVLSQSPAILSASPGEKVTMTCRASSSVSYMHWYQQKPGSSPKPWIYAPSNLASGVPARFSGSGSGTSYSLTISRVEAEDAATYYCQQWSFNPPTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 20 - protein, 253 aa, Artificial sequence
  sequence is synthesized
MKKNIAFLLASMFVFSIATNAYAQAYLQQSGAELVRPGASVKMSCKASGYTFTSYNMHWVKQTPRQGLEWIGAIYPGNGDTSYNQKFKGKATLTVDKSSSTAYMQLSSLTSEDSAVYFCARVVYYSNSYWYFDVWGTGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHT

SEQ ID NO: 21 - DNA, 5391 bp, Homo sapiens
ttcgagctcgcccgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggccgggaacggtgcattggaacgcggattccccgtgccaagagtgacgtaagtaccgcctatagagtctataggcccacccccttggcttcgttagaacgcggctacaattaatacataaccttatgtatcatacacatacgatttaggtgacactatagaataacatccactttgcctttctctccacaggtgtccactcccaggtccaactgcacctcggttctatcgattgaattccaccatgggatggtcatgtatcatcctttttctagtagcaactgcaactggagtacattcagatatccagatgacccagtccccgagctccctgtccgcctctgtgggcgatagggtcaccatcacctgccgtgccagtcaggacatccgtaattatttgaactggtatcaacagaaaccaggaaaagctccgaaactactgatttactatacctcccgcctggagtctggagtcccttctcgcttctctggttctggttctgggacggattacactctgaccatcagtagtctgcaaccggaggacttcgcaacttattactgtcagcaaggtaatactctgccgtggacgttcggacagggcaccaaggtggagatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttaagcttggccgccatggcccaacttgtttattgcagcttataatggttacaaataaagcaatagcatcacaaatttcacaaataaagcatttttttcactgcattctagttgtggtttgtccaaactcatcaatgtatcttatcatgtctggatcgatcgggaattaattcggcgcagcaccatggcctgaaataacctctgaaagaggaacttggttaggtaccttctgaggcggaaagaaccagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagctgttaacagcttggcactggccgtcgttttacaacgtcgtgactgggaaaaccctggcgttacccaacttaatcgccttgcagcacatccccccttcgccagctggcgtaatagcgaagaggcccgcaccgatcgcccttcccaacagttgcgtagcctgaatggcgaatggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccaactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagtattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgatgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgccagcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatccagctggcacgacaggtttcccgactggaaagcgggcagtgagcgcaacgcaattaatgtgagttacctcactcattaggcaccccaggctttacactttatgcttccggctcgtatgttgtgtggaattgtgagcggataacaatttcacacaggaaacagctatgaccatgattacgaattaa

SEQ ID NO: 22 - DNA, 6135 bp, Homo sapiens
attcgagctcgcccgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggccgggaacggtgcattggaacgcggattccccgtgccaagagtgacgtaagtaccgcctatagagtctataggcccacccccttggcttcgttagaacgcggctacaattaatacataaccttatgtatcatacacatacgatttaggtgacactatagaataacatccactttgcctttctctccacaggtgtccactcccaggtccaactgcacctcggttctatcgattgaattccaccatgggatggtcatgtatcatcctttttctagtagcaactgcaactggagtacattcagaagttcagctggtggagtctggcggtggcctggtgcagccagggggctcactccgtttgtcctgtgcagcttctggctactcctttaccggctacactatgaactgggtgcgtcaggccccaggtaagggcctggaatgggttgcactgattaatccttataaaggtgttactacctatgccgatagcgtcaagggccgtttcactataagcgtagataaatccaaaaacacagcctacctgcaaatgaacagcctgcgtgctgaggacactgccgtctattattgtgctagaagcggatactacggcgatagcgactggtattttgacgtctggggtcaaggaaccctggtcaccgtctcctcggcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgactgtgccctctagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaagagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatgagtgcgacggccctagagtcgacctgcagaagcttggccgccatggcccaacttgtttattgcagcttataatggttacaaataaagcaatagcatcacaaatttcacaaataaagcatttttttcactgcattctagttgtggtttgtccaaactcatcaatgtatcttatcatgtctggatcgatcgggaattaattcggcgcagcaccatggcctgaaataacctctgaaagaggaacttggttaggtaccttctgaggcggaaagaaccatctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagctgttaacagcttggcactggccgtcgttttacaacgtcgtgactgggaaaaccctggcgttacccaacttaatcgccttgcagcacatccccccttcgccagttggcgtaatagcgaagaggcccgcaccgatcgcccttcccaacagttgcgtagcctgaatggcgaatggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccaactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagtattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgatgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgccagcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatccaactggcacgacaggtttcccgactggaaagcgggcagtgagcgcaacgcaattaatgtgagttacctcactcattaggcaccccaggctttacactttatgcttccggctcgtatgttgtgtggaattgtgagcggataacaatttcacacaggaaacagctatgaccatgattacgaatta

SEQ ID NO: 23 - protein, 232 aa, Artificial sequence
  sequence is synthesized
MGWSCIILFLVATATGVHSDIQMTQSPSSLSASVGDRVTITCRASSSVSYMHWYQQKPGKAPKPLIYAPSNLASGVPSRFSGSGSGTDFTLTISSLQPEDFATYYCQQWSFNPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 24 - protein, 213 aa, Artificial sequence
  sequence is synthesized
DIQMTQSPSSLSASVGDRVTITCRASSSVSYMHWYQQKPGKAPKPLIYAPSNLASGVPSRFSGSGSGTDFTLTISSLQPEDFATYYCQQWSFNPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 25 - protein, 471 aa, Artificial sequence
  sequence is synthesized
MGWSCIILFLVATATGVHSEVQLVESGGGLVQPGGSLRLSCAASGYTFTSYNMHWVRQAPGKGLEWVGAIYPGNGDTSYNQKFKGRFTISVDKSKNTLYLQMNSLRAEDTAVYYCARVVYYSNSYWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 26 - protein, 452 aa, Artificial sequence
  sequence is synthesized
EVQLVESGGGLVQPGGSLRLSCAASGYTFTSYNMHWVRQAPGKGLEWVGAIYPGNGDTSYNQKFKGRFTISVDKSKNTLYLQMNSLRAEDTAVYYCARVVYYSNSYWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 27 - protein, 471 aa, Artificial sequence
  sequence is synthesized
MGWSCIILFLVATATGVHSEVQLVESGGGLVQPGGSLRLSCAASGYTFTSYNMHWVRQAPGKGLEWVGAIYPGNGDTSYNQKFKGRFTISVDKSKNTLYLQMNSLRAEDTAVYYCARVVYYSNSYWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNATYRVVSVLTVLHQDWLNGKEYKCKVSNKALPAPIAATISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 28 - protein, 452 aa, Artificial sequence
  sequence is synthesized
EVQLVESGGGLVQPGGSLRLSCAASGYTFTSYNMHWVRQAPGKGLEWVGAIYPGNGDTSYNQKFKGRFTISVDKSKNTLYLQMNSLRAEDTAVYYCARVVYYSNSYWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNATYRVVSVLTVLHQDWLNGKEYKCKVSNKALPAPIAATISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 29 - protein, 213 aa, Artificial sequence
  sequence is synthesized
DIQMTQSPSSLSASVGDRVTITCRASSSVSYLHWYQQKPGKAPKPLIYAPSNLASGVPSRFSGSGSGTDFTLTISSLQPEDFATYYCQQWAFNPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 30 - protein, 452 aa, Artificial sequence
  sequence is synthesized
EVQLVESGGGLVQPGGSLRLSCAASGYTFTSYNMHWVRQAPGKGLEWVGAIYPGNGATSYNQKFKGRFTISVDKSKNTLYLQMNSLRAEDTAVYYCARVVYYSASYWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNATYRVVSVLTVLHQDWLNGKEYKCKVSNAALPAPIAATISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 31 - DNA, 36 bp, Artificial sequence
  sequence is synthesized
ctacaccttcacgagctataacatgcactgggtccg

SEQ ID NO: 32 - DNA, 36 bp, Artificial sequence; identical to SEQ ID NO: 31
  sequence is synthesized

SEQ ID NO: 33 - DNA, 38 bp, Artificial sequence
  sequence is synthesized
gaatgggttgcagcgatctatcctggcaacggcgacac

SEQ ID NO: 34 - DNA, 65 bp, Artificial sequence
  sequence is synthesized
attattgtgctcgagtggtctactatagcaacagctactggtacttcgacgtctggggtcaagga

SEQ ID NO: 35 - DNA, 36 bp, Artificial sequence
  sequence is synthesized
ctgcacagccagctcttctgtcagctatatgcattg

SEQ ID NO: 36 - DNA, 42 bp, Artificial sequence
  sequence is synthesized
aactactgatttacgctccatcgaacctcgcgtctggagtcc

SEQ ID NO: 37 - DNA, 45 bp, Artificial sequence
  sequence is synthesized
tattactgtcaacagtggagcttcaatccgcccacatttggacag

SEQ ID NO: 38 - DNA, 37 bp, Artificial sequence
  sequence is synthesized
gtttcactataagtgtcgacaagtccaaaaacacatt

SEQ ID NO: 39 - DNA, 33 bp, Artificial sequence
  sequence is synthesized
gccaggatagatggcgccaacccattccaggcc

SEQ ID NO: 40 - DNA, 26 bp, Artificial sequence
  sequence is synthesized
aagctccgaaaccactgatttacgct

SEQ ID NO: 41 - DNA, 18 bp, Artificial sequence
  sequence is synthesized
agttttgagagcaaaatg

SEQ ID NO: 42 - DNA, 18 bp, Artificial sequence
  sequence is synthesized
aagctatgaacactaatg

SEQ ID NO: 43 - protein, 107 aa, Artificial sequence
  sequence is synthesized
DIQMTQSPSSLSASVGDRVTITCRASSSVSYLHWYQQKPGKAPKPLIYAPSNLASGVPSRFSGSGSGTDFTLTISSLQPEDFATYYCQQWAFNPPTFGQGTKVEIKR

SEQ ID NO: 44 - protein, 213 aa, Artificial sequence; identical to SEQ ID NO: 29
  sequence is synthesized

SEQ ID NO: 45 - protein, 452 aa, Artificial sequence
  sequence is synthesized
EVQLVESGGGLVQPGGSLRLSCAASGYTFTSYNMHWVRQAPGKGLEWVGAIYPGNGATSYNQKFKGRFTISVDKSKNTLYLQMNSLRAEDTAVYYCARVVYYSYRYWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNATYRVVSVLTVLHQDWLNGKEYKCKVSNAALPAPIAATISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

(19) United States
(12) Patent Application Publication
(10) Pub. No.: US 2006/0002930 A1
(43) Pub. Date: Jan. 5, 2006

(54) Treatment of disorders

(75) Inventors: Paul G. Brunetta, San Francisco, CA (US); Kathryn L. Sewell, San Francisco, CA (US)

Correspondence Address: GENENTECH, INC., 1 DNA WAY, SOUTH SAN FRANCISCO, CA, 94080, US

(73) Assignee: GENENTECH, INC.

(21) Appl. No.: 11/106,820

(22) Filed: Apr. 15, 2005

Related U.S. Application Data
(60) Provisional application No. 60/565,098, filed on Apr. 22, 2004.
(60) Provisional application No. 60/563,227, filed on Apr. 16, 2004.

(51) Int. Cl. A61K 39/395; A61K 51/00; A61K 31/525; A61K 31/66

(52) U.S. Cl. 424/144.1; 424/1.49; 514/11; 514/109; 514/251

(57) ABSTRACT

The present invention concerns treatment of polychondritis or mononeuritis multiplex in a mammal with an effective amount of an antibody that binds to CD20, optionally also with another agent that treats such disorders in an effective amount.

### RELATED APPLICATIONS

This application claims priority to U.S. Provisional Application Nos.: 60/563,227 filed Apr. 16, 2004 and 60/565,098 filed Apr. 22, 2004, to which U.S. Provisional Applications this application claims priority under 35 U.S.C. § 119, the contents of which are incorporated herein by reference.

### FIELD OF THE INVENTION

The present invention concerns treatment of disorders with antagonists that bind to B-cell surface markers, such as CD19 or CD20, e.g. antibodies that bind to CD20.

### BACKGROUND OF THE INVENTION

Lymphocytes are one of many types of white blood cells produced in the bone marrow during the process of hematopoiesis. There are two major populations of lymphocytes: B lymphocytes (B cells) and T lymphocytes (T cells). The lymphocytes of particular interest herein are B cells.

B cells mature within the bone marrow and leave the marrow expressing an antigen-binding antibody on their cell surface. When a naive B cell first encounters the antigen for which its membrane-bound antibody is specific, the cell begins to divide rapidly and its progeny differentiate into memory B cells and effector cells called “plasma cells.” Memory B cells have a longer life span and continue to express membrane-bound antibody with the same specificity as the original parent cell. Plasma cells do not produce membrane-bound antibody but instead produce the antibody in a form that can be secreted. Secreted antibodies are the major effector molecule of humoral immunity.

The CD20 antigen (also called human B-lymphocyte-restricted differentiation antigen, Bp35) is a hydrophobic transmembrane protein with a molecular weight of approximately 35 kD located on pre-B and mature B lymphocytes (Valentine et al. J. Biol. Chem. 264(19):11282-11287 (1989); and Einfeld et al. EMBO J. 7(3):711-717 (1988)). The antigen is also expressed on greater than 90% of B-cell non-Hodgkin's lymphomas (NHL) (Anderson et al. Blood 63(6): 1424-1433 (1984)), but is not found on hematopoietic stem cells, pro-B cells, normal plasma cells or other normal tissues (Tedder et al. J. Immunol. 135(2):973-979 (1985)). CD20 regulates an early step(s) in the activation process for cell-cycle initiation and differentiation (Tedder et al., supra) and possibly functions as a calcium ion channel (Tedder et al. J. Cell. Biochem. 14D: 195 (1990)).

Given the expression of CD20 in B-cell lymphomas, this antigen can serve as a candidate for “targeting” of such lymphomas. In essence, such targeting can be generalized as follows: antibodies specific to the CD20 surface antigen of B cells are administered to a patient. These anti-CD20 antibodies specifically bind to the CD20 antigen of (ostensibly) both normal and malignant B cells; the antibody bound to the CD20 surface antigen may lead to the destruction and depletion of neoplastic B cells. Additionally, chemical agents or radioactive labels having the potential to destroy the tumor can be conjugated to the anti-CD20 antibody such that the agent is specifically “delivered” to the neoplastic B cells. Irrespective of the approach, a primary goal is to destroy the tumor; the specific approach can be determined by the particular anti-CD20 antibody which is utilized and, thus, the available approaches to targeting the CD20 antigen can vary considerably.

CD19 is another antigen that is expressed on the surface of cells of the B lineage. Like CD20, CD19 is found on cells throughout differentiation of the lineage from the stem cell stage up to a point just prior to terminal differentiation into plasma cells (Nadler, L. Lymphocyte Typing II 2: 3-37 and Appendix, Renling et al. eds. (1986) by Springer Verlag). Unlike CD20, however, antibody binding to CD19 causes internalization of the CD19 antigen. CD19 antigen is identified by the HD237-CD19 antibody (also called the “AB4” antibody) (Kiesel et al. Leukemia Research II, 12: 1119 (1987)), among others. The CD19 antigen is present on 4-8% of peripheral blood mononuclear cells and on greater than 90% of B cells isolated from peripheral blood, spleen, lymph node or tonsil. CD19 is not detected on peripheral blood T cells, monocytes, or granulocytes. Virtually all non-T-cell acute lymphoblastic leukemias (ALL), B-cell chronic lymphocytic leukemias (CLL) and B-cell lymphomas express CD19 detectable by the antibody B4 (Nadler et al. J. Immunol. 131:244 (1983); and Nadler et al. in Progress in Hematology Vol. XII pp. 187-206, Brown, E. ed. (1981) by Grune & Stratton, Inc.).

Additional antibodies that recognize differentiation stage-specific antigens expressed by cells of the B-cell lineage have been identified. Among these are the B2 antibody directed against the CD21 antigen; B3 antibody directed against the CD22 antigen; and the J5 antibody directed against the CD10 antigen (also called CALLA). See, e.g., U.S. Pat. No. 5,595,721 issued Jan. 21, 1997 (Kaminski et al.).

The rituximab (RITUXAN®) antibody is a genetically engineered chimeric murine/human monoclonal antibody directed against the CD20 antigen. Rituximab is the antibody called “AC2B8” in U.S. Pat. No. 5,736,137 issued Apr. 7, 1998 (Anderson et al.). RITUXAN® is indicated for the treatment of patients with relapsed or refractory low-grade or follicular, CD20 positive, B-cell non-Hodgkin's lymphoma (Maloney et al. Blood 82 (Suppl 1): 445a (1993); Maloney et al. Proc Am Soc Clin Oncol 13: 993 (1994)). In vitro mechanism of action studies have demonstrated that RITUXAN® binds human complement and lyses lymphoid B-cell lines through complement-dependent cytotoxicity (CDC) (Reff et al. Blood 83(2):435-445 (1994)). Additionally, it has significant activity in assays for antibody-dependent cellular cytotoxicity (ADCC). More recently, RITUXAN® has been shown to have anti-proliferative effects in tritiated thymidine incorporation assays and to induce apoptosis directly, while other anti-CD19 and CD20 antibodies do not (Maloney et al. Blood 88(10):637a (1996)). Synergy between RITUXAN® and chemotherapies and toxins has also been observed experimentally. In particular, RITUXAN® sensitizes drug-resistant human B-cell lymphoma cell lines to the cytotoxic effects of doxorubicin, CDDP, VP-16, diphtheria toxin and ricin (Demidem et al. Cancer Chemotherapy & Radiopharmaceuticals 12(3): 177-186 (1997); Demidem A et al. FASEB J 9:A206 (1995)). In vivo preclinical studies have shown that RITUXAN® depletes B cells from the peripheral blood, lymph nodes, and bone marrow of cynomolgus monkeys, presumably through complement and cell-mediated processes (Reff et al., supra).

Rituximab has also been studied in a variety of non-malignant autoimmune disorders, in which B cells and autoantibodies appear to play a role in disease pathophysiology. Edwards et al., Biochem Soc. Trans. 30:824-828 (2002). Rituximab has been reported to potentially relieve signs and symptoms of, for example, rheumatoid arthritis (RA) (Leandro et al., Ann. Rheum. Dis. 61:883-888 (2002); Edwards et al., Arthritis Rheum., 46 (Suppl. 9): S46 (2002); Stahl et al., Ann. Rheum. Dis., 62 (Suppl. 1): OP004 (2003); Emery et al., Arthritis Rheum. 48(9): S439 (2003)), lupus (Eisenberg, Arthritis. Res. Ther. 5:157-159 (2003); Leandro et al. Arthritis Rheum. 46: 2673-2677 (2002); Gorman et al., Lupus, 13: 312-316 (2004)), immune thrombocytopenic purpura (D'Arena et al., Leuk. Lymphoma 44:561-562 (2003); Stasi et al., Blood, 98: 952-957 (2001); Saleh et al., Semin. Oncol., 27 (Supp 12):99-103 (2000); Zaia et al., Haematolgica, 87: 189-195 (2002); Ratanatharathorn et al., Ann. Int. Med., 133: 275-279 (2000)), pure red cell aplasia (Auner et al., Br. J. Haematol., 116: 725-728 (2002)); autoimmune anemia (Zaja et al., Haematologica 87:189-195 (2002) (erratum appears in Haematologica 87:336 (2002)), cold agglutinin disease (Layios et al., Leukemia, 15: 187-8 (2001); Berentsen et al., Blood, 103: 2925-2928 (2004); Berentsen et al., Br. J. Haematol., 115: 79-83 (2001); Bauduer, Br. J. Haematol., 112: 1083-1090 (2001); Damiani et al., Br. J. Haematol., 114: 229-234 (2001)), type B syndrome of severe insulin resistance (Coll et al., N. Engl. J. Med., 350: 310-311 (2004), mixed cryoglobulinemia (DeVita et al., Arthritis Rheum. 46 Suppl. 9:S206/S469 (2002)), myasthenia gravis (Zaja et al., Neurology, 55: 1062-63 (2000); Wylam et al., J. Pediatr., 143: 674-677 (2003)), Wegener's granulomatosis (Specks et al., Arthritis & Rheumatism 44: 2836-2840 (2001)), refractory pemphigus vulgaris (Dupuy et al., Arch Dermatol., 140:91-96 (2004)), dermatomyositis (Levine, Arthritis Rheum., 46 (Suppl. 9):S1299 (2002)), Sjögren's syndrome (Somer et al., Arthritis & Rheumatism, 49: 394-398 (2003)), active type-II mixed cryoglobulinemia (Zaja et al., Blood, 101: 3827-3834 (2003)), pemphigus vulgaris (Dupay et al., Arch. Dermatol., 140: 91-95 (2004)), autoimmune neuropathy (Pestronk et al., J. Neurol. Neurosurg. Psychiatry 74:485-489 (2003)), paraneoplastic opsoclonus-myoclonus syndrome (Pranzatelli et al. Neurology 60(Suppl. 1) PO5.128:A395 (2003)), and relapsing-remitting multiple sclerosis (RRMS). Cross et al. (abstract) “Preliminary Results from a Phase II Trial of Rituximab in MS” Eighth Annual Meeting of the Americas Committees for Research and Treatment in Multiple Sclerosis, 20-21 (2003).

A Phase II study (WA16291) has been conducted in patients with rheumatoid arthritis (RA), providing 48-week follow-up data on safety and efficacy of Rituximab. Emery et al. Arthritis Rheum 48(9):S439 (2003); Szczepanski et al. Arthritis Rheum 48(9):S 121 (2003). A total of 161 patients were evenly randomized to four treatment arms: methotrexate, rituximab alone, rituximab plus methotrexate, and rituximab plus cyclophosphamide (CTX). The treatment regimen of rituximab was one gram administered intravenously on days 1 and 15. Infusions of rituximab in most patients with RA were well tolerated by most patients, with 36% of patients experiencing at least one adverse event during their first infusion (compared with 30% of patients receiving placebo). Overall, the majority of adverse events was considered to be mild to moderate in severity and was well balanced across all treatment groups. There were a total of 19 serious adverse events across the four arms over the 48 weeks, which were slightly more frequent in the rituximab/CTX group. The incidence of infections was well balanced across all groups. The mean rate of serious infection in this RA patient population was 4.66 per 100 patient-years, which is lower than the rate of infections requiring hospital admission in RA patients (9.57 per 100 patient-years) reported in a community-based epidemiologic study. Doran et al., Arthritis Rheum. 46:2287-2293 (2002).

The reported safety profile of rituximab in a small number of patients with neurologic disorders, including autoimrnune neuropathy (Pestronk et al., supra), opsoclonus-myoclonus syndrome (Pranzatelli et al., supra), and RRMS (Cross et al., supra), was similar to that reported in oncology or RA. In an ongoing investigator-sponsored trial (IST) of rituximab in combination with interferon-beta (IFN-β) or glatiramer acetate in patients with RRMS (Cross et al., supra), 1 of 10 treated patients was admitted to the hospital for overnight observation after experiencing moderate fever and rigors following the first infusion of rituximab, while the other 9 patients completed the four-infusion regimen without any reported adverse events.

Patents and patent publications concerning CD20 antibodies and CD20 binding molecules include U.S. Pat. Nos. 5,776,456, 5,736,137, 5,843,439, 6,399,061, and 6,682,734, as well as U.S. 2002/0197255, U.S. 2003/0021781, U.S. 2003/0082172, U.S. 2003/0095963, U.S. 2003/0147885 (Anderson et al.); U.S. Pat. No. 6,455,043 and WO 2000/09160 (Grillo-Lopez, A.); WO 2000/27428 (Grillo-Lopez and White); WO 2000/27433 (Grillo-Lopez and Leonard); WO 2000/44788 (Braslawsky et al.); WO 2001/10462 (Rastetter, W.); WO01/10461 (Rastetter and White); WO 2001/10460 (White and Grillo-Lopez); U.S. 2001/0018041, U.S. 2003/0180292, WO 2001/34194 (Hanna and Hariharan); U.S. 2002/0006404 and WO 2002/04021 (Hanna and Hariharan); U.S. 2002/0012665 and WO 2001n4388 (Hanna, N.); U.S. 2002/0058029 (Hanna, N.); U.S. 2003/0103971 (Hariharan and Hanna); U.S. 2002/0009444 and WO 2001/80884 (Grillo-Lopez, A.); WO 2001/97858 (White, C.); U.S. 2002/0128488 and WO 2002/34790 (Reff, M.); WO 2002/060955 (Braslawsky et al.); WO 2002/096948 (Braslawsky et al.); WO 2002/079255 (Reff and Davies); U.S. Pat. No. 6,171,586 and WO 1998/56418 (Lam et al.); WO 1998/58964 (Raju, S.); WO 1999/22764 (Raju, S.); WO 1999/51642, U.S. Pat. No. 6,194,551, U.S. Pat. No. 6,242,195, U.S. Pat. No. 6,528,624 and U.S. Pat. No. 6,538,124 (Idusogie et al.); WO 2000/42072 (Presta, L.); WO 2000/67796 (Curd et al.); WO 2001/03734 (Grillo-Lopez et al.); U.S. 2002/0004587 and WO 2001n7342 (Miller and Presta); U.S. 2002/0197256 (Grewal, I.); U.S. 2003/0157108 (Presta, L.); U.S. Pat. Nos. 6,565,827, 6,090,365, 6,287,537, 6,015,542, 5,843,398, and 5,595,721, (Kaminski et al.); U.S. Pat. Nos. 5,500,362, 5,677,180, 5,721,108, 6,120,767, and 6,652,852 (Robinson et al.); U.S. Pat. No. 6,410,391 (Raubitschek et al.); U.S. Pat. No. 6,224,866 and WO00/20864 (Barbera-Guillem, E.); WO 2001/13945 (Barbera-Guillem, E.); WO 2000/67795 (Goldenberg); U.S. 2003/0133930 and WO 2000/74718 (Goldenberg and Hansen); U.S. 2003/0219433 and WO 2003/68821 (Hansen et al.); WO2004/058298 (Goldenberg and Hansen); WO 2000/76542 (Golay et al.); WO 2001/72333 (Wolin and Rosenblatt); U.S. Pat. No. 6,368,596 (Ghetie et al.); U.S. Pat. No. 6,306,393 and U.S. 2002/0041847 (Goldenberg, D.); U.S. 2003/0026801 (Weiner and Hartmann); WO 2002/102312 (Engleman, E.); U.S. 2003/0068664 (Albitar et al.); WO 2003/002607 (Leung, S.); WO 2003/049694, U.S. 2002/0009427, and U.S. 2003/0185796 (Wolin et al.); WO 2003/061694 (Sing and Siegall); U.S. 2003/0219818 (Bohen et al.); U.S. 2003/0219433 and WO 2003/068821 (Hansen et al.); U.S. 2003/0219818 (Bohen et al.); U.S. 2002/0136719 (Shenoy et al.); WO 2004/032828 (Wahl et al.); and WO 2002/56910 (Hayden-Ledbetter). See also U.S. Pat. No. 5,849,898 and EP 330,191 (Seed et al.); EP332,865A2 (Meyer and Weiss); U.S. Pat. No. 4,861,579 (Meyer et al.); U.S. 2001/0056066 (Bugelski et al.); WO 1995/03770 (Bhat et al.); U.S. 2003/0219433 A1 (Hansen et al.); WO 2004/035607 (Teeling et al.); WO 2004/056312 (Lowman et al.); U.S. 2004/0093621 (Shitara et al.); WO 2004/103404 (Watkins et al.); WO 2005/000901 (Tedder et al.); U.S. 2005/0025764 (Watkins et al.); WO 2005/016969 (Carr et al.); and U.S. 2005/0069545 (Carr et al.). WO 2004/032828 mentions relapsing polychondritis as one of a list of immune disorders to be treated with anti-CD20 antibodies.

Publications concerning therapy with rituximab include: Perotta and Abuel, “Response of chronic relapsing ITP of 10 years duration to rituximab” Abstract # 3360 Blood 10(1)(part 1-2): p. 88B (1998); Perotta et al., “Rituxan in the treatment of chronic idiopathic thrombocytopaenic purpura (ITP)”, Blood, 94: 49 (abstract) (1999); Matthews, R., “Medical Heretics” New Scientist (7 Apr., 2001); Leandro et al., “Clinical outcome in 22 patients with rheumatoid arthritis treated with B lymphocyte depletion” Ann Rheum Dis, supra; Leandro et al., “Lymphocyte depletion in rheumatoid arthritis: early evidence for safety, efficacy and dose response” Arthritis and Rheumatism 44(9): S370 (2001); Leandro et al., “An open study of B lymphocyte depletion in systemic lupus erythematosus”, Arthritis and Rheumatism, 46:2673-2677 (2002), wherein during a 2-week period, each patient received two 500-mg infusions of rituximab, two 750-mg infusions of cyclophosphamide, and high-dose oral corticosteroids, and wherein two of the patients treated relapsed at 7 and 8 months, respectively, and have been retreated, although with different protocols; “Successful long-term treatment of systemic lupus erythematosus with rituximab maintenance therapy” Weide et al., Lupus, 12: 779-782 (2003), wherein a patient was treated with rituximab (375 mg/m²×4, repeated at weekly intervals) and further rituximab applications were delivered every 5-6 months and then maintenance therapy was received with rituximab 375 mg/m² every three months, and a second patient with refractory SLE was treated successfully with rituximab and is receiving maintenance therapy every three months, with both patients responding well to rituximab therapy; Edwards and Cambridge, “Sustained improvement in rheumatoid arthritis following a protocol designed to deplete B lymphocytes” Rheumatology 40:205-211 (2001); Cambridge et al., “B lymphocyte depletion in patients with rheumatoid arthritis: serial studies of immunological parameters” Arthritis Rheum., 46 (Suppl. 9): S1350 (2002); Edwards et al., “B-lymphocyte depletion therapy in rheumatoid arthritis and other autoimmune disorders” Biochem Soc. Trans., supra; Edwards et al., “Efficacy and safety of rituximab, a B-cell targeted chimeric monoclonal antibody: A randomized, placebo controlled trial in patients with rheumatoid arthritis. Arthritis and Rheumatism 46(9): S197 (2002); Edwards et al., “Efficacy of B-cell-targeted therapy with rituximab in patients with rheumatoid arthritis” N Engl. J. Med. 350:2572-82 (2004); Pavelka et al., Ann. Rheum. Dis. 63: (S 1):289-90 (2004); Emery et al., Arthritis Rheum. 50 (S9):S659 (2004); Levine and Pestronk, “IgM antibody-related polyneuropathies: B-cell depletion chemotherapy using rituximab” Neurology 52: 1701-1704 (1999); DeVita et al., “Efficacy of selective B cell blockade in the treatment of rheumatoid arthritis” Arthritis & Rheum 46:2029-2033 (2002); Hidashida et al. “Treatment of DMARD-refractory rheumatoid arthritis with rituximab.” Presented at the Annual Scientific Meeting of the American College of Rheumatology; Oct. 24-29; New Orleans, La. 2002; Tuscano, J. “Successful treatment of infliximab-refractory rheumatoid arthritis with rituximab” Presented at the Annual Scientific Meeting of the American College of Rheumatology; Oct. 24-29; New Orleans, La. 2002; “Pathogenic roles of B cells in human autoimmunity; insights from the clinic” Martin and Chan, Immunity 20:517-527 (2004); Silverman and Weisman, “Rituximab Therapy and Autoimmune Disorders, Prospects for Anti-B Cell Therapy”, Arthritis and Rheumatism, 48: 1484-1492 (2003); Kazkaz and Isenberg, “Anti B cell therapy (rituximab) in the treatment of autoimmune diseases”, Current opinion in pharmacology, 4: 398402 (2004); Virgolini and Vanda, “Rituximab in autoimmune diseases”, Biomedicine & pharmacotherapy, 58: 299-309(2004); Klemmer et al., “Treatment of antibody mediated autoimmune disorders with a AntiCD20 monoclonal antibody Rituximab”, Arthritis And Rheumatism, 48: (9) 9,S (SEP), page: S624-S624 (2003); Kneitz et al., “Effective B cell depletion with rituximab in the treatment of autoimmune diseases”, Immunobiology, 206: 519-527 (2002); Arzoo et al., “Treatment of refractory antibody mediated autoimmune disorders with an anti-CD20 monoclonal antibody (rituximab)” Annals of the Rheumatic Diseases, 61 (10), p922-4 (2002) Comment in Ann Rheum Dis. 61: 863-866 (2002); “Future Strategies in Immunotherapy” by Lake and Dionne, in Burger's Medicinal Chemistry and Drug Discovery (2003 by John Wiley & Sons, Inc.) Article Online Posting Date: Jan. 15, 2003 (Chapter 2 “Antibody-Directed Immunotherapy”); Liang and Tedder, Wiley Encyclopedia of Molecular Medicine, Section: CD20 as an Immunotherapy Target, article online posting date: 15 Jan., 2002 entitled “CD20”; Appendix 4A entitled “Monoclonal Antibodies to Human Cell Surface Antigens” by Stockinger et al., eds: Coligan et al., in Current Protocols in Immunology (2003 John Wiley & Sons, Inc) Online Posting Date: May, 2003; Print Publication Date: February, 2003; Penichet and Morrison, “CD Antibodies/molecules: Definition; Antibody Engineering” in Wiley Encyclopedia of Molecular Medicine Section: Chimeric, Humanized and Human Antibodies; posted online 15 Jan., 2002; Specks et al. “Response of Wegener's granulomatosis to anti-CD20 chimeric monoclonal antibody therapy” Arthritis & Rheumatism 44:2836-2840 (2001); online abstract submission and invitation Koegh et al., “Rituximab for Remission Induction in Severe ANCA-Associated Vasculitis: Report of a Prospective Open-Label Pilot Trial in 10 Patients”, American College of Rheumatology, Session Number: 28-100, Session Title: Vasculitis, Session Type: ACR Concurrent Session, Primary Category: 28 Vasculitis, Session Oct. 18, 2004 (<www.abstractsonline.com/viewer/SearchResults.asp>); Eriksson, “Short-term outcome and safety in 5 patients with ANCA-positive vasculitis treated with rituximab”, Kidney and Blood Pressure Research, 26: 294 (2003); Jayne et al., “B-cell depletion with rituximab for refractory vasculitis” Kidney and Blood Pressure Research, 26: 294 (2003); Jayne, poster 88 (11^{th} International Vasculitis and ANCA workshop), 2003 American Society of Nephrology; Stone and Specks, “Rituximab Therapy for the Induction of Remission and Tolerance in ANCA-associated Vasculitis”, in the Clinical Trial Research Summary of the 2002-2003 Immune Tolerance Network, <www.immunetolerance.org/research/autoimmune/trials/stone.html>. See also Leandro et al., “B cell repopulation occurs mainly from naive B cells in patient with rheumatoid arthritis and systemic lupus erythematosus” Arthritis Rheum., 48 (Suppl 9): S1160 (2003).

Sarwal et al. N. Eng. J. Med. 349(2):125-138 (Jul. 10, 2003) reports molecular heterogeneity in acute renal allograft rejection identified by DNA microarray profiling.

Relapsing polychondritis is an uncommon, chronic disorder of the cartilage that is characterized by recurrent episodes of inflammation of the cartilage of various tissues of the body. Tissues containing cartilage that can become inflamed include the ears, nose, joints, spine, and windpipe (trachea). The eyes, heart, and blood vessels, which have a biochemical makeup similar to that of cartilage, can also be affected.

The cause of relapsing polychondritis is unknown. It is suspected that this condition is caused by an immune system disorder (autoimmunity) in which the body's immunity system (which normally fights off invaders of the body, particularly infections) is misguided. This results in inflammation that is directed at various tissues of the body. Relief can be found through anti-inflammatory agents and various steroids.

Mononeuritis multiplex is a painful asymmetric asynchronous sensory and motor peripheral neuropathy involving isolated damage to at least two separate nerve areas. Multiple nerves in random areas of the body can be affected. As the condition worsens, it becomes less multifocal and more symmetric, resembling polyneuropathy. Mononeuropathy multiplex syndromes can be distributed bilaterally, distally, and proximally throughout the body. The damage to the nerves involves destruction of the axon (i.e., the part of the nerve cell that is analogous to the copper part of a wire), thus interfering with nerve conduction at the location of the damage. Common causes include diabetes and multiple nerve compressions, as well as a lack of oxygen caused by decreased blood flow or inflammation of blood vessels. No cause is identified for about one-third of cases. Multiple specific disorders are associated with mononeuritis multiplex, including (but not limited to) blood vessel diseases such as polyarteritis nodosa and other vasculitic diseases, diabetes, and connective tissue diseases such as rheumatoid arthritis or systemic lupus erythematosus. Connective tissue disease is the most common cause in children. Less common causes include the following: Sjögren's syndrome, Wegener's granulomatosis, hypersensitivity (allergic reactions) that causes inflammation of blood vessels, leprosy, sarcoidosis, amyloidosis, multifocal forms of diabetic neuropathy, and disorders of the blood (such as hypereosinophilia and cryoglobulinemia). See, for example, Hattori et al. Brain 122(3):427-439 (1999) wherein the clinicopathological features of 28 patients with peripheral neuropathy associated with Churg-Strauss syndrome were assessed, and sensory and motor involvement mostly showed a pattern of mononeuritis multiplex in the initial phase, progressing into asymmetrical polyneuropathy, restricted to the limbs. CD20-positive B lymphocytes were seen only occasionally.

The treatment for neuropathy depends on its cause, and many neuropathies can be treated by addressing the underlying cause (such as vitamin deficiency). Others can be prevented from occurring. For example, controlling diabetes may prevent diabetic neuropathy. In cases where a tumor or ruptured disc is the cause, therapy may involve surgery to remove the tumor or to repair the ruptured disc. In entrapment or compression neuropathy treatment may consist of splinting or surgical decompression of the ulnar or median nerves. Peroneal and radial compression neuropathies may require avoidance of pressure. Physical therapy and/or splints may be useful in preventing contractures (a condition in which shortened muscles around joints cause abnormal and sometimes painful positioning of the joints). Neuropathies that are associated with immune diseases can improve with treatment directed at the abnormal features of the immune system. Such treatments include intravenous immunoglobulin, plasma exchange and immunosuppressive therapy (Cook et al. Neurology 40:212-214 (1990); Dyck et al. N. Engl. J. Med 325:1482-1486 (1991); Ernerudh et al. J. Neurol. Neurosurg. Psychiatry 55:930-934 (1992); Blume et al. Neurology 45:1577-1580 (1995); Pestronk et al. Neurology 44:2027-2031 (1994)). These may produce minimal functional improvement. Moreover, the treatment can be expensive and time consuming.

The literature in antibody-directed treatment against B-cell surface membrane markers is extremely limited. Levine and Pestronk described five patients with neuropathy and immunoglobulin M antibodies to GM1 or MAG who were treated with rituximab. Within 3-6 months of treatment all five had improved function and reduced titer of serum antibodies (Levine and Pestornk Am. J. Neurol. 52:1701-1704 (1999)).

If a specific treatment is not available, the pain of the neuropathy can usually be controlled, such as with the use of analgesics, pain medication, tricyclic antidepressants, anti-seizure medications, or a nerve blocker.

Sutton and Winer Current Opinion in Pharmacology 2/3:291-295 (Jun. 1, 2002) state that plasma exchange, intravenous immunoglobulin and corticosteroids continue to be the mainstay of treatment for inflammatory neuropathies. Recent trials demonstrate that combining these therapies is not significantly more effective than single-agent treatment. The usefulness of novel immunotherapies and cytotoxic agents is difficult to ascertain because of the treatment of small numbers of patients in open-label studies.

Lee et al. Bone Marrow Transplantation 30/1:53-56 (2002) proposes that high-dose chemotherapy and autologous peripheral blood stem cell (PBSC) transplantation may have a role in the treatment of peripheral neuropathy secondary to severe, progressive and treatment-resistant monoclonal gammopathy of unknown significance (MGUS). Latov et al. Neurology 52:A551 (1999) discloses that RITUXAN® appeared to be safe and effective treatment in two patients with neuropathy-associated with IgM monoclonal gammopathy and anti-MAG antibody activity. Canavan et al. Neurology 58/7 (Suppl. 3):A233 (April 2002) disclosed that RITUXAN® was associated with sustained clinical improvement in the majority of patients treated that exhibited IgM autoantibody-associated polyneuropathy.

Regarding monoclonal antibody treatment of mixed cryoglobulinemia resistant to interferon-alpha with an anti-CD20 antibody, Sansonno et al. Blood 101(10):3818-3826 (May 15, 2003) discloses treatment of peripheral neuropathy with RITUXAN®.

Hattori et al. Brain 122/3:427-439 (1999) assessed the clinicopathological features of patients with peripheral neuropathy associated with Churg-Strauss syndrome, stating that CD20-positive B lymphocytes were seen only occasionally.

Zaja et al. Blood 101 (10):3827-3834 (May 15, 2003) disclosed that RITUXAN® may represent a safe and effective alternative to standard immunosuppression in type II mixed cryoglobulinemia (MC). RITUXAN® proved effective on skin vasculitis manifestations (ulcers, purpura, or urticaria), subjective symptoms of peripheral neuropathy, low-grade B-cell lymphoma, arthralgias, and fever. Zaidi et al. Leukemia and Lymphoma 45/4:777-780 (2004) disclosed that a case of lymphomatoid granulomatosis (LYG), a rare lymphoproliferative disorder with a mortality rate approaching 60% in the first year, with pulmonary, hepatic, central and peripheral nervous system involvement, was successfully treated with RITUXAN®. Yet, Trojan et al. Annals of Oncology 13/5:802-805 (2002) disclosed that RITUXAN® did not appear to be effective for a patient suffering from peripheral neuropathy due to neurolymphomatosis. Fused PET-CT imaging, performed on an in-line PET-CT system, showed multiple small nodular lesions extending along the peripheral nerves corresponding to an early relapse of a transformed B-cell non-Hodgkin's lymphoma.

Binstadt et al. Journal of Pediatrics 143/5:598-604 (November 2003) concluded that RITUXAN® was safe and effective in four pediatric patients with multisystem autoimmune diseases refractory to conventional immunosuppressive medications, each with central nervous system (CNS) involvement. One patient with autoimmune cytopenias and autoimmune CNS and peripheral nervous system disease had resolution of the cytopenias and marked improvement in neurologic symptoms; they report that he currently receives no immunosuppressive medications. Two half-siblings with lymphoplasmacytic colitis, pulmonary nodules, and CNS disease had improvement of their symptoms. A fourth patient with chorea and seizures secondary to primary antiphospholipid antibody syndrome had improvement in fine and gross motor function and reduced seizure frequency. Saito et al. Lupus 12/10:798-800 (2003) discloses that RITUXAN® was useful in treating a patient with refractory lupus nephritis and CNS involvement of systemic lupus erythematosus (SLE) associated with highly active B lymphocytes.

There exists a need in the art for additional drugs to treat various indications such as polychondritis and mononeuritis multiplex.

### SUMMARY OF THE INVENTION

Accordingly, the invention is as claimed. Specifically, the present invention provides, in a first aspect, a method of treating polychondritis or mononeuritis multiplex in a mammal comprising administering to the mammal an effective amount of an antibody that binds CD20.

In one embodiment of this method, the antibody is not conjugated with another molecule. In another embodiment, the antibody is conjugated with another molecule, for example, a cytotoxic agent such as a radioactive compound, e.g., Y2B8 or ¹³¹I-B1. In another embodiment, the antibody comprises rituximab or humanized 2H7. The humanized 2H7 in one embodiment comprises the variable domain sequences in SEQ ID Nos. 2 and 8. In another embodiment, the humanized 2H7 comprises a variable heavy-chain domain with alteration(s) N100A or D56A,N100A in SEQ ID NO:8 and a variable light-chain domain with alteration(s) M32L, S92A, or M32L,S92A in SEQ ID NO:2. In a further embodiment, the humanized 2H7 comprises the light-chain variable region (V_{L}) sequence of SEQ ID NO:30 and the heavy-chain variable region (V_{H}) sequence of SEQ ID NO:8, wherein the antibody further contains an amino acid substitution of D56A in VH-CDR2, and N100 in VH-CDR3 is substituted with Y or W, and more preferably the antibody comprises the v511 light-chain sequence of SEQ ID NO:31 and the v511 heavy-chain sequence of SEQ ID NO:32.

The antibody is preferably administered in a dose of about 20 mg/m² to about 250 mg/m² of the antibody to the mammal, more preferably, about 50 mg/m² to about 200 mg/m². In another preferred embodiment, the method comprises administering an initial dose of the antibody followed by a subsequent dose, wherein the mg/m² dose of the antibody in the subsequent dose exceeds the mg/m² dose of the antibody in the initial dose.

In yet another preferred embodiment, the mammal is human. The antibody is preferably administered intravenously or subcutaneously.

In a preferred embodiment, the method consists essentially of administering an effective amount of the antibody to the mammal.

In another preferred aspect, the method further comprises administering to the mammal an effective amount of an immunosuppressive agent, anti-pain agent, or chemotherapeutic agent.

In still further embodiments, if polychondritis is treated, the method further comprises administering to the mammal an effective amount of a non-steroidal anti-inflammatory drug, steroid, or immunosuppressive agent such as methotrexate, cyclophosphamide, dapsone, azathioprine, penicillamine, or cyclosporine. If mononeuritis multiplex is treated, the method further comprises administering to the mammal an effective amount of an anti-pain agent, steroid, methotrexate, cyclophosphamide, plasma exchange, intravenous immunoglobulin, cyclosporine, or mycophenolate mofetil.

In a further aspect, the present invention pertains to an article of manufacture comprising a container and a composition contained therein, wherein the composition comprises an antibody that binds CD20, and further comprising a package insert instructing the user of the composition to treat polychondritis or mononeuritis multiplex in a mammal. In a preferred embodiment, the article further comprises a container comprising an agent other than the antibody for the treatment and further comprising instructions on treating the mammal with such agent.

### BRIEF DESCRIPTION OF THE DRAWINGS

FIG. 1A is a sequence alignment comparing the amino acid sequences of the light-chain variable domain (V_{L}) of each of murine 2H7 (SEQ ID NO:1), humanized 2H7.v16 variant (SEQ ID NO:2), and the human kappa light-chain subgroup I (SEQ ID NO:3). The CDRs of V_{L} of 2H7 and hu2H7.v16 are as follows: CDR1 (SEQ ID NO:4), CDR2 (SEQ ID NO:5), and CDR3 (SEQ ID NO:6).

FIG. 1B is a sequence alignment comparing the amino acid sequences of the heavy-chain variable domain (V_{H}) of each of murine 2H7 (SEQ ID NO:7), humanized 2H7.v16 variant (SEQ ID NO:8), and the human consensus sequence of the heavy-chain subgroup III (SEQ ID NO:9). The CDRs of V_{H} of 2H7 and hu2H7.v 16 are as follows: CDR1 (SEQ ID NO:10), CDR2 (SEQ ID NO:11), and CDR3 (SEQ ID NO:12).

In FIG. 1A and FIG. 1B, the CDR1, CDR2 and CDR3 in each chain are enclosed within brackets, flanked by the framework regions, FR1-FR4, as indicated. 2H7 refers to the murine 2H7 antibody. The asterisks in between two rows of sequences indicate the positions that are different between the two sequences. Residue numbering is according to Kabat et al. Sequences of Immunological Interest, 5th Ed. Public Health Service, National Institutes of Health, Bethesda, Md. (1991), with insertions shown as a, b, c, d, and e.

FIG. 2 shows the nucleotide sequence of phagemid pVX4 (SEQ ID NO:13 {5′ sequence}) and SEQ ID NO:14 {3′ complementary sequence}) used for construction of 2H7Fab plasmids (see Example 1) as well as the amino acid sequences of the L chain (SEQ ID NO:15) and H chain (SEQ ID NO:16) of the Fab for the CDR-grafted anti-IFN-α humanized antibody.

FIG. 3 shows the nucleotide sequence of the expression plasmid that encodes the chimeric 2H7.v6.8 Fab (SEQ ID NO:17 {5′ sequence} and SEQ ID NO:18 {3′ complementary sequence}). The amino acid sequences of the L chain (SEQ ID NO:19) and H chain (SEQ ID NO:20) are shown.

FIG. 4 shows the nucleotide sequence of the plasmid pDR1 (SEQ ID NO:21; 5391 bp) for expression of immunoglobulin light chains as described in Example 1. pDR1 contains sequences encoding an irrelevant antibody, the light chain of a humanized anti-CD3 antibody (Shalaby et al. J. Exp. Med. 175:217-225 (1992)), the start and stop codons for which are indicated in bold and underlined.

FIG. 5 shows the nucleotide sequence of plasmid pDR2 (SEQ ID NO:22; 6135 bp) for expression of immunoglobulin heavy chains as described in Example 1. pDR2 contains sequences encoding an irrelevant antibody, the heavy chain of a humanized anti-CD3 antibody (Shalaby et al., supra), the start and stop codons for which are indicated in bold and underlined.

FIGS. 6A and 6B show the amino acid sequences of the 2H7.v16 L chain, with FIG. 6A showing the complete L chain containing the first 19 amino acids before DIQ that are the secretory signal sequence not present in the mature polypeptide chain (SEQ ID NO:23), and FIG. 6B showing the mature polypeptide L chain (SEQ ID NO:24)

FIGS. 7A and 7B show the amino acid sequences of the 2H7.v16H chain, with FIG. 7A showing the complete H chain containing the first 19 amino acids before EVQ that are the secretory signal sequence not present in the mature polypeptide chain (SEQ ID NO:25), and FIG. 7B showing the mature polypeptide H chain (SEQ ID NO:26). Aligning the V_{H} sequence in FIG. 1B (SEQ ID NO:8) with the complete H chain sequence, the human γ1 constant region is from amino acid position 114-471 in SEQ ID NO:25.

FIGS. 8A and 8B show the amino acid sequences of the 2H7.v31H chain, with FIG. 8A showing the complete H chain containing the first 19 amino acids before EVQ that are the secretory signal sequence not present in the mature polypeptide chain (SEQ ID NO:27), and FIG. 8B showing the mature polypeptide H chain (SEQ ID NO:28). The L chain is the same as for 2H7.v16 (see FIG. 6).

FIG. 9 is a flow chart summarizing the amino acid changes from the murine 2H7 to a subset of humanized versions up to v75.

FIG. 10 is a sequence alignment comparing the light-chain amino acid sequences of the humanized 2H7.v16 variant (SEQ ID NO:2) and humanized 2H7.v138 variant (SEQ ID NO:29).

FIG. 11 is a sequence alignment comparing the heavy-chain amino acid sequences of the humanized 2H7.v16 variant (SEQ ID NO:8) and humanized 2H7.v138 variant (SEQ ID NO:30).

### DETAILED DESCRIPTION OF THE PREFERRED EMBODIMENTS

I. Definitions

A “B-cell surface marker” or “B-cell surface antigen” herein is an antigen expressed on the surface of a B cell that can be targeted with an antagonist that binds thereto. Exemplary B-cell surface markers include the CD10, CD19, CD20, CD21, CD22, CD23, CD24, CD37, CD40, CD53, CD72, CD73, CD74, CDw75, CDw76, CD77, CDw78, CD79a, CD79b, CD80, CD81, CD82, CD83, CDw84, CD85 and CD86 leukocyte surface markers. (For descriptions, see The Leukocyte Antigen Facts Book, 2^{nd} Edition. 1997, ed. Barclay et al. Academic Press, Harcourt Brace & Co., New York). Other B-cell surface markers include RP105, FcRH2, CD79A, C79B, B cell CR2, CCR6, CD72, P2×5, HLA-DOB, CXCR5, FCER2, BR3, Btig, NAG14, SLGC16270, FcRH1, IRTA2, ATWD578, FcRH3, IRTA1, FcRH6, BCMA, and 239287_at. The B-cell surface marker of particular interest is preferentially expressed on B cells compared to other non-B-cell tissues of a mammal and may be expressed on both precursor B cells and mature B cells. The preferred B-cell surface markers herein are CD20 and CD22.

The “CD20” antigen, or “CD20,” is an about 35-kDa, non-glycosylated phosphoprotein found on the surface of greater than 90% of B cells from peripheral blood or lymphoid organs. CD20 is present on both normal B cells as well as malignant B cells, but is not expressed on stem cells. Other names for CD20 in the literature include “B-lymphocyte-restricted antigen” and “Bp35”. The CD20 antigen is described in Clark et al. Proc. Natl. Acad. Sci. (USA) 82:1766 (1985), for example.

The “CD22” antigen, or “CD22,” also known as BL-CAM or Lyb8, is a type 1 integral membrane glycoprotein with molecular weight of about 130 (reduced) to 140 kD (unreduced). It is expressed in both the cytoplasm and cell membrane of B-lymphocytes. CD22 antigen appears early in B-cell lymphocyte differentiation at approximately the same stage as the CD19 antigen. Unlike other B-cell markers, CD22 membrane expression is limited to the late differentiation stages comprised between mature B cells (CD22+) and plasma cells CD22-). The CD22 antigen is described, for example, in Wilson et al. J. Exp. Med. 173:137 (1991) and Wilson et al. J. Immunol. 150:5013 (1993).

A “non-malignant disorder” herein is polychondritis or mononeuritis multiplex, preferably mononeuritis multiplex. Additionally, it may be spino-optical multiple sclerosis; pemphigus vulgaris; Churg-Strauss vasculitis or syndrome (CSS); lupus cerebritis; lupus nephritis; cutaneous systemic lupus erythematosus (SLE); IgE-mediated diseases other than asthma, specifically, allergic rhinitis, anaphylaxis, or atopic dermatitis; chronic neuropathy; opsoclonus-myoclonus syndrome; pulmonary alveolar proteinosis; scleritis; microscopic polyangiitis; paraneoplastic syndrome, which is a remote effect produced by a tumor, such as hypercalcemia, but not including Lambert-Eaton, anemia, or hypoglycemia; Rasmussen's encephalitis; central nervous system (CNS) vasculitis; channelopathies, which are diseases with diverse properties associated with ion channel dysfunction such as epilepsy, migraine, arrhythmia, muscular disorders, deafness, blindness, periodic paralysis, and channelopathies of the CNS, but not including CNS inflammatory disorders; autism; or neuropathic, myopathic, or CNS sarcoidosis.

“Polychondritis” as used herein means any polychondritis, including relapsing polychondritis, Von Meyenburg disease, Meyenburg disease or syndrome, Meyenburg Altherr Uehlinger syndrome, polychondropathy, Askenazy, Jaksch Wartenhorst, Meyenburg, or Von Jaksch Wartenhorst syndrome, perichondritis that is chondrolytic, diffuse or relapsing, chondromalacic arthritis, or panchondritis.

“Mononeuritis multiplex” as used herein describes a condition characterized by inflammation caused by several nerves in unrelated portions of the body, i.e., the nerve damage involves isolated damage to at least two separate nerve areas. As it worsens, it may become more diffuse and less focused on particular areas, resembling polyneuropathy. The symptoms of a disease of this sort may include numbness, weakness, burning pain (especially at night), and loss of reflexes. The pain may be severe and disabling.

An “antagonist” is a molecule that, upon binding to a B-cell surface marker, destroys or depletes B cells in a mammal and/or interferes with one or more B-cell functions, e.g. by reducing or preventing a humoral response elicited by the B cell. The antagonist preferably is able to deplete B cells (i.e. reduce circulating B-cell levels) in a mammal treated therewith. Such depletion may be achieved via various mechanisms such as ADCC and/or CDC, inhibition of B-cell proliferation, and/or induction of B-cell death (e.g. via apoptosis). Antagonists included within the scope of the present invention include antibodies, synthetic or native-sequence peptides and small-molecule antagonists that bind to the B-cell marker, optionally conjugated with or fused to a cytotoxic agent. The preferred antagonist comprises an antibody, i.e., an antibody that binds a B-cell surface marker.

“Antibody-dependent cell-mediated cytotoxicity” and “ADCC” refer to a cell-mediated reaction in which nonspecific cytotoxic cells that express Fc receptors (FcRs) (e.g. Natural Killer (NK) cells, neutrophils, and macrophages) recognize bound antibody on a target cell and subsequently cause lysis of the target cell. The primary cells for mediating ADCC, NK cells, express FcγRIII only, whereas monocytes express FcγRI, FcγRII and FcγRIII. FcR expression on hematopoietic cells is summarized in Table 3 on page 464 of Ravetch and Kinet, Annu. Rev. Immunol 9:457-92 (1991). To assess ADCC activity of a molecule of interest, an in vitro ADCC assay, such as that described in U.S. Pat. Nos. 5,500,362 or 5,821,337, may be performed. Useful effector cells for such assays include peripheral blood mononuclear cells (PBMC) and NK cells. Alternatively, or additionally, ADCC activity of the molecule of interest may be assessed in vivo, e.g., in a animal model such as that disclosed in Clynes et al. PNAS (USA) 95:652-656 (1998).

“Human effector cells” are leukocytes that express one or more FcRs and perform effector functions. Preferably, the cells express at least FcγRIII and carry out ADCC effector function. Examples of human leukocytes that mediate ADCC include PBMC, NK cells, monocytes, cytotoxic T cells and neutrophils, with PBMCs and NK cells being preferred.

The terms “Fc receptor” or “FcR” are used to describe a receptor that binds to the Fc region of an antibody. The preferred FcR is a native-sequence human FcR. Moreover, a preferred FcR is one that binds an IgG antibody (a gamma receptor) and includes receptors of the FcγRI, FcγRII, and Fcγ RIII subclasses, including allelic variants and alternatively spliced forms of these receptors. FcγRII receptors include FcγRIIA (an “activating receptor”) and FcγRIIB (an “inhibiting receptor”), which have similar amino acid sequences that differ primarily in the cytoplasmic domains thereof. Activating receptor FcγRIIA contains an immunoreceptor tyrosine-based activation motif (ITAM) in its cytoplasmic domain. Inhibiting receptor FcγRIIB contains an immunoreceptor tyrosine-based inhibition motif (ITIM) in its cytoplasmic domain. (See Daëron, Annu. Rev. Immunol. 15:203-234 (1997)). FcRs are reviewed in Ravetch and Kinet, Annu. Rev. Immunol 9:457-92 (1991); Capel et al. Immunomethods 4:25-34 (1994); and de Haas et al. J. Lab. Clin. Med. 126:33041 (1995). Other FcRs, including those to be identified in the future, are encompassed by the term “FcR” herein. The term also includes the neonatal receptor, FcRn, which is responsible for the transfer of maternal IgGs to the fetus (Guyer et al. J. Immunol. 117:587 (1976) and Kim et al. J. Immunol. 24:249 (1994)).

“Complement-dependent cytotoxicity” or “CDC” refers to the ability of a molecule to lyse a target in the presence of complement. The complement activation pathway is initiated by the binding of the first component of the complement system (C1q) to a molecule (e.g. an antibody) complexed with a cognate antigen. To assess complement activation, a CDC assay, e.g. as described in Gazzano-Santoro et al. J. Immunol. Methods 202:163 (1996), may be performed.

“Growth-inhibitory” antagonists are those that prevent or reduce proliferation of a cell expressing an antigen to which the antagonist binds. For example, the antagonist may prevent or reduce proliferation of B cells in vitro and/or in vivo.

Antagonists that “induce apoptosis” are those that induce programmed cell death, e.g. of a B cell, as determined by standard apoptosis assays, such as binding of annexin V, fragmentation of DNA, cell shrinkage, dilation of endoplasmic reticulum, cell fragmentation, and/or formation of membrane vesicles (called apoptotic bodies).

The term “antibody” herein is used in the broadest sense and specifically covers intact monoclonal antibodies, polyclonal antibodies, multispecific antibodies (e.g. bispecific antibodies) formed from at least two intact antibodies, and antibody fragments so long as they exhibit the desired biological activity.

“Antibody fragments” comprise a portion of an intact antibody, preferably comprising the antigen-binding or variable region thereof. Examples of antibody fragments include Fab, Fab′, F(ab′)₂, and Fv fragments; diabodies; linear antibodies; single-chain antibody molecules; and multispecific antibodies formed from antibody fragments.

For the purposes herein, an “intact antibody” is one comprising heavy- and light-chain variable domains as well as an Fc region.

“Native antibodies” are usually heterotetrameric glycoproteins of about 150,000 daltons, composed of two identical light (L) chains and two identical heavy (H) chains. Each light chain is linked to a heavy chain by one covalent disulfide bond, while the number of disulfide linkages varies among the heavy chains of different immunoglobulin isotypes. Each heavy and light chain also has regularly spaced intrachain disulfide bridges. Each heavy chain has at one end a variable domain (V_{H}) followed by a number of constant domains. Each light chain has a variable domain at one end (V_{L}) and a constant domain at its other end; the constant domain of the light chain is aligned with the first constant domain of the heavy chain, and the light-chain variable domain is aligned with the variable domain of the heavy chain. Particular amino acid residues are believed to form an interface between the light-chain and heavy-chain variable domains.

The term “variable” refers to the fact that certain portions of the variable domains differ extensively in sequence among antibodies and are used in the binding and specificity of each particular antibody for its particular antigen. However, the variability is not evenly distributed throughout the variable domains of antibodies. It is concentrated in three segments called hypervariable regions both in the light-chain and the heavy-chain variable domains. The more highly conserved portions of variable domains are called the framework regions (FRs). The variable domains of native heavy and light chains each comprise four FRs, largely adopting a β-sheet configuration, connected by three hypervariable regions, which form loops connecting, and in some cases forming part of, the β-sheet structure. The hypervariable regions in each chain are held together in close proximity by the FRs and, with the hypervariable regions from the other chain, contribute to the formation of the antigen-binding site of antibodies (see Kabat et al. Sequences of Proteins of Immunological Interest, 5th Ed. Public Health Service, National Institutes of Health, Bethesda, Md. (1991)). The constant domains are not involved directly in binding an antibody to an antigen, but exhibit various effector functions, such as participation of the antibody in ADCC.

Papain digestion of antibodies produces two identical antigen-binding fragments, called “Fab” fragments, each with a single antigen-binding site, and a residual “Fc” fragment, whose name reflects its ability to crystallize readily. Pepsin treatment yields an F(ab′)₂ fragment that has two antigen-binding sites and is still capable of cross-linking antigen.

“Fv” is the minimum antibody fragment that contains a complete antigen-recognition and antigen-binding site. This region consists of a dimer of one heavy-chain and one light-chain variable domain in tight, non-covalent association. It is in this configuration that the three hypervariable regions of each variable domain interact to define an antigen-binding site on the surface of the V_{H}-V_{L} dimer. Collectively, the six hypervariable regions confer antigen-binding specificity to the antibody. However, even a single variable domain (or half of an Fv comprising only three hypervariable regions specific for an antigen) has the ability to recognize and bind antigen, although at a lower affinity than the entire binding site.

The Fab fragment also contains the constant domain of the light chain and the first constant domain (CH1) of the heavy chain. Fab′ fragments differ from Fab fragments by the addition of a few residues at the carboxy terminus of the heavy-chain CH1 domain including one or more cysteines from the antibody hinge region. Fab′-SH is the designation herein for Fab′ in which the cysteine residue(s) of the constant domains bear at least one free thiol group. F(ab′)₂ antibody fragments originally were produced as pairs of Fab′ fragments that have hinge cysteines between them. Other chemical couplings of antibody fragments are also known.

The “light chains” of antibodies (immunoglobulins) from any vertebrate species can be assigned to one of two clearly distinct types, called kappa (κ) and lambda (λ), based on the amino acid sequences of their constant domains.

Depending on the amino acid sequence of the constant domain of their heavy chains, antibodies can be assigned to different classes. There are five major classes of intact antibodies: IgA, IgD, IgE, IgG, and IgM, and several of these may be further divided into subclasses (isotypes), e.g., IgG1, IgG2, IgG3, IgG4, IgA, and IgA2. The heavy-chain constant domains that correspond to the different classes of antibodies are called α, δ, ε, γ, and μ, respectively. The subunit structures and three-dimensional configurations of different classes of immunoglobulins are well known.

“Single-chain Fv” or “scFv” antibody fragments comprise the V_{H} and V_{L} domains of antibody, wherein these domains are present in a single polypeptide chain. Preferably, the Fv polypeptide further comprises a polypeptide linker between the V_{H} and V_{L} domains that enables the scFv to form the desired structure for antigen binding. For a review of scFv, see Plückthun in The Pharmacology of Monoclonal Antibodies, vol. 113, Rosenburg and Moore eds., Springer-Verlag, New York, pp. 269-315 (1994).

The term “diabodies” refers to small antibody fragments with two antigen-binding sites, which fragments comprise a heavy-chain variable domain (V_{H}) connected to a light-chain variable domain (V_{L}) in the same polypeptide chain (V_{H}-V_{L}). By using a linker that is too short to allow pairing between the two domains on the same chain, the domains are forced to pair with the complementary domains of another chain and create two antigen-binding sites. Diabodies are described more fully in, for example, EP 404,097; WO 93/11161; and Hollinger et al. Proc. Natl. Acad. Sci. USA, 90:6444-6448 (1993).

The term “monoclonal antibody” as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Furthermore, in contrast to conventional (polyclonal) antibody preparations that typically include different antibodies directed against different determinants (epitopes), each monoclonal antibody is directed against a single determinant on the antigen. In addition to their specificity, the monoclonal antibodies are advantageous in that they are synthesized by the hybridoma culture, uncontaminated by other immunoglobulins. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by the hybridoma method first described by Kohler et al. Nature, 256:495 (1975), or may be made by recombinant DNA methods (see, e.g., U.S. Pat. No. 4,816,567). The “monoclonal antibodies” may also be isolated from phage antibody libraries using the techniques described in Clackson et al. Nature, 352:624-628 (1991) and Marks et al. J. Mol. Biol., 222:581-597 (1991), for example.

The monoclonal antibodies herein specifically include “chimeric” antibodies (immunoglobulins) in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (U.S. Pat. No. 4,816,567; Morrison et al. Proc. Natl. Acad. Sci. USA, 81:6851-6855 (1984)). Chimeric antibodies of interest herein include “primatized” antibodies comprising variable domain antigen-binding sequences derived from a non-human primate (e.g. Old World Monkey, such as baboon, rhesus or cynomolgus monkey) and human constant-region sequences (U.S. Pat. No. 5,693,780).

“Humanized” forms of non-human (e.g., murine) antibodies are chimeric antibodies that contain minimal sequence derived from non-human immunoglobulin. For the most part, humanized antibodies are human immunoglobulins (recipient antibody) in which residues from a hypervariable region of the recipient are replaced by residues from a hypervariable region of a non-human species (donor antibody) such as mouse, rat, rabbit or non-human primate having the desired specificity, affinity, and capacity. In some instances, framework region (FR) residues of the human immunoglobulin are replaced by corresponding non-human residues. Furthermore, humanized antibodies may comprise residues that are not found in the recipient antibody or in the donor antibody. These modifications are made to further refine antibody performance. In general, the humanized antibody will comprise substantially all of at least one, and typically two, variable domains, in which all or substantially all of the hypervariable loops correspond to those of a non-human immunoglobulin and all or substantially all of the FRs are those of a human immunoglobulin sequence. The humanized antibody optionally also will comprise at least a portion of an immunoglobulin constant region (Fc), typically that of a human immunoglobulin. For further details, see Jones et al. Nature 321:522-525 (1986); Riechmann et al. Nature 332:323-329 (1988); and Presta, Curr. Op. Struct. Biol. 2:593-596 (1992).

The term “hypervariable region” when used herein refers to the amino acid residues of an antibody that are responsible for antigen binding. The hypervariable region comprises amino acid residues from a “complementarity-determining region” or “CDR” (e.g. residues 24-34 (L1), 50-56 (L2) and 89-97 (L3) in the light-chain variable domain and 31-35 (H1), 50-65 (H2) and 95-102 (H3) in the heavy-chain variable domain; Kabat et al. Sequences of Proteins of Immunological Interest, 5th Ed. Public Health Service, National Institutes of Health, Bethesda, Md. (1991)) and/or those residues from a “hypervariable loop” (e.g. residues 26-32 (L1), 50-52 (L2) and 91-96 (L3) in the light-chain variable domain and 26-32 (H1), 53-55 (H2) and 96-101 (H3) in the heavy-chain variable domain; Chothia and Lesk J. Mol. Biol. 196:901-917 (1987)). “Framework” or “FR” residues are those variable domain residues other than the hypervariable region residues as herein defined.

An antagonist “that binds” an antigen of interest, e.g. a B-cell surface marker, is one capable of binding that antigen with sufficient affinity and/or avidity such that the antagonist is useful as a therapeutic agent for targeting a cell expressing the antigen.

Examples of antibodies that bind the CD19 antigen include the anti-CD19 antibodies in Hekman et al. Cancer Immunol. Immunother. 32:364-372 (1991) and Vlasveld et al. Cancer Immunol. Immunother. 40:37-47 (1995); and the B4 antibody in Kiesel et al. Leukemia Research II, 12: 1119 (1987).

An “antibody that binds CD20” refers to an antibody that binds CD20 antigen with sufficient affinity and/or avidity such that the antibody is useful as a therapeutic agent for targeting a cell expressing or overexpressing CD20 antigen. Examples of such antibodies include: “C2B8” which is now called “rituximab” (“RITUXAN®”) (U.S. Pat. No. 5,736,137); the yttrium-[90]-labeled 2B8 murine antibody designated “Y2B8” or “Ibritumomab Tiuxetan” ZEVALIN® (U.S. Pat. No. 5,736,137); murine IgG2a “B1,” also called “Tositumomab,” optionally labeled with ¹³¹I to generate the “¹³¹I-B1” antibody (iodine I131 tositumomab, BEXXAR™) (U.S. Pat. No. 5,595,721); murine monoclonal antibody “1F5” (Press et al. Blood 69(2):584-591 (1987) and “framework patched” or humanized 1F5 (WO03/002607, Leung, S.); ATCC deposit HB-96450); murine 2H7 and chimeric 2H7 antibody (U.S. Pat. No. 5,677,180); a humanized 2H7; huMax-CD20 (Genmab, Denmark); AME-133 (Applied Molecular Evolution); and monoclonal antibodies L27, G28-2, 93-1B3, B-C1 or NU-B2 available from the International Leukocyte Typing Workshop (Valentine et al., In: Leukocyte Typing III (McMichael, Ed., p. 440, Oxford University Press (1987)).

The terms “rituximab” and “RITUXAN®” herein refer to the genetically engineered chimeric murine/human monoclonal antibody directed against the CD20 antigen and designated “C2B8” in U.S. Pat. No. 5,736,137, including fragments thereof that retain the ability to bind CD20.

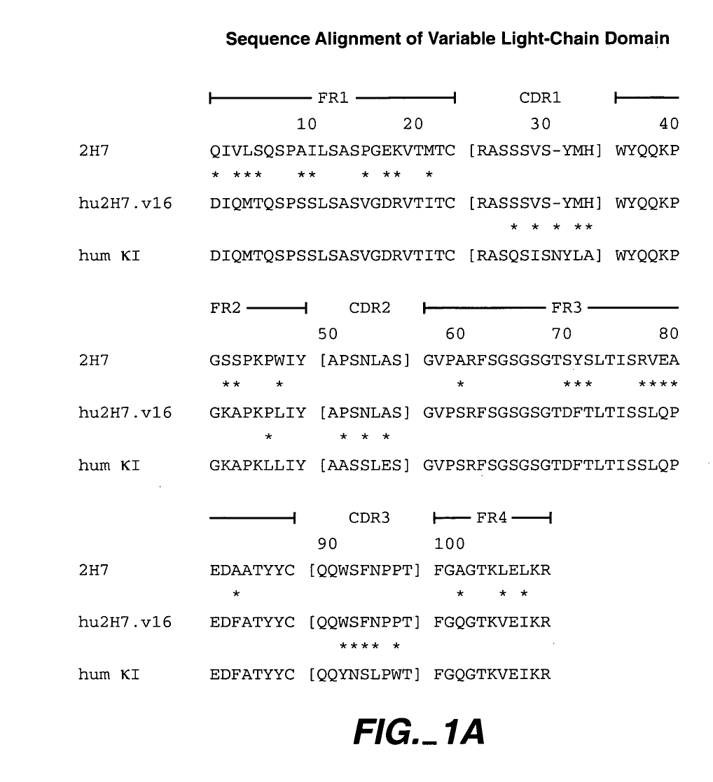

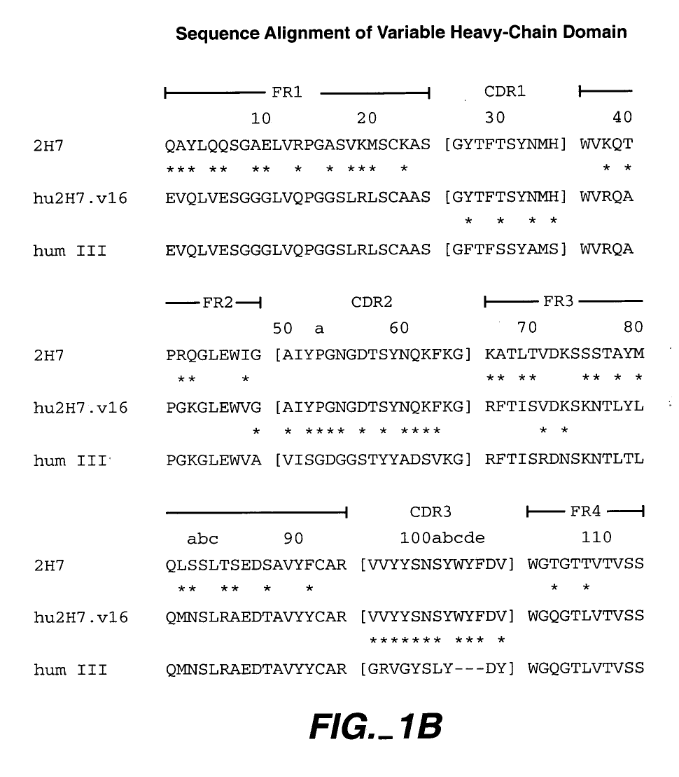

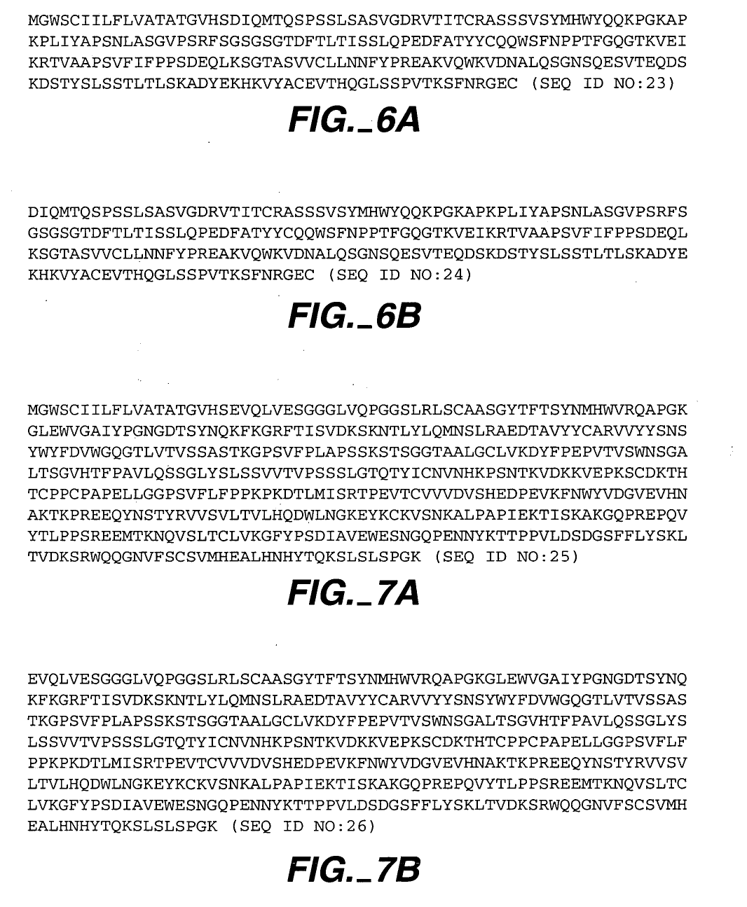

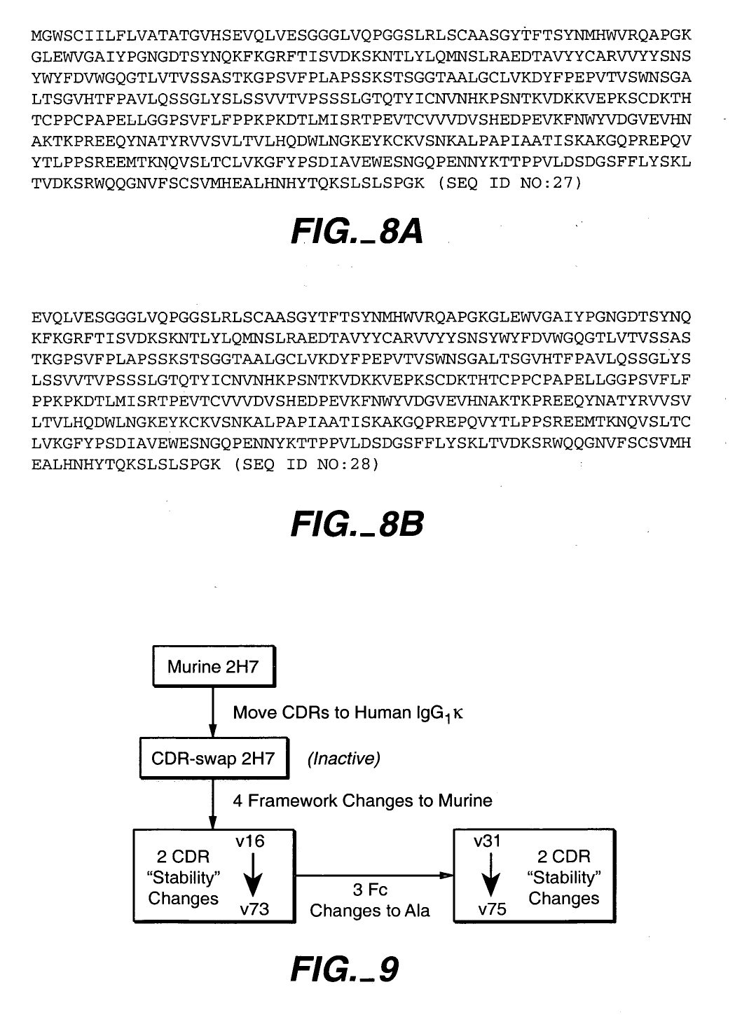

Purely for the purposes herein, “humanized 2H7” refers to a humanized antibody that binds human CD20, or an antigen-binding fragment thereof, wherein the antibody is effective to deplete primate B cells in vivo, the antibody comprising in the H chain variable region (V_{H}) at least a CDR3 sequence of SEQ ID NO:12 (FIG. 1B) from an anti-human CD20 antibody and substantially the human consensus framework (FR) residues of the human heavy-chain subgroup III (V_{H}III). In a preferred embodiment, this antibody further comprises the H chain CDR1 sequence of SEQ ID NO:10 and CDR2 sequence of SEQ ID NO:11, and more preferably further comprises the L chain CDR1 sequence of SEQ ID NO:4, CDR2 sequence of SEQ ID NO:5, CDR3 sequence of SEQ ID NO:6 and substantially the human consensus framework (FR) residues of the human light-chain κ subgroup I (VκI), wherein the V_{H} region may be joined to a human IgG chain constant region, wherein the region may be, for example, IgG1 or IgG3. In a preferred embodiment, such antibody comprises the V_{H} sequence of SEQ ID NO:8 (v16, as shown in FIG. 1B), optionally also comprising the V_{L} sequence of SEQ ID NO:2 (v16, as shown in FIG. 1A), which may have the amino acid substitutions of D56A and N100A in the H chain and S92A in the L chain (v.96). A more preferred such antibody is 2H7.v16 having the light- and heavy-chain amino acid sequences of SEQ ID NOS:24 and 26, respectively, as shown in FIGS. 6B and 7B. Another preferred embodiment is where the antibody is 2H7.v31 having the light- and heavy-chain amino acid sequences of SEQ ID NOS:24 and 28, respectively, as shown in FIGS. 6B and 8B. The antibody herein may further comprise at least one amino acid substitution in the Fc region that improves ADCC and/or CDC activity, such as one wherein the amino acid substitutions are S298A/E333A/K334A, more preferably 2H7.v31 having the heavy-chain amino acid sequence of SEQ ID NO:28 (as shown in FIG. 8B). Any of these antibodies may further comprise at least one amino acid substitution in the Fc region that decreases CDC activity, for example, comprising at least the substitution K322A. Such antibodies preferably are 2H7.v114 or 2H7.v115 having at least 10-fold improved ADCC activity as compared to RITUXAN®.

A preferred humanized 2H7 is an intact antibody or antibody fragment comprising the variable light-chain sequence:

(SEQ ID NO:2)

DIQMTQSPSSLSASVGDRVTITCRASSSVSYMHWYQQKPGKAPKPLIYAP
SNLASGVPSRFSGSGSGTDFTLTISSLQPEDFATYYCQQWSFNPPTFGQG
TKVEIKR;

and the variable heavy-chain sequence:

(SEQ ID NO:8)

EVQLVESGGGLVQPGGSLRLSCAASGYTFTSYNMHWVRQAPGKGLEWVGA
IYPGNGDTSYNQKFKGRFTISVDKSKNTLYLQMNSLRAEDTAVYYCARVV
YYSNSYWYFDVWGQGTLVTVSS.

Where the humanized 2H7 antibody is an intact antibody, preferably it comprises the light-chain amino acid sequence:

(SEQ ID NO:24)

DIQMTQSPSSLSASVGDRVTITCRASSSVSYMHWYQQKPGKAPKPLIYAP
SNLASGVPSRFSGSGSGTDFTLTISSLQPEDFATYYCQQWSFNPPTFGQG
TKVEIKRTVAAPSVFIFPPSDEQLKSGTASVVCLLNNFYPREAKVQWKVD
NALQSGNSQESVTEQDSKDSTYSLSSTLTLSKADYEKHKVYACEVTHQGL
SSPVTKSFNRGEC;

and the heavy-chain amino acid sequence:

(SEQ ID NO:26)

EVQLVESGGGLVQPGGSLRLSCAASGYTFTSYNMHWVRQAPGKGLEWVGA
IYPGNGDTSYNQKFKGRFTISVDKSKNTLYLQMNSLRAEDTAVYYCARVV
YYSNSYWYFDVWGQGTLVTVSSASTKGPSVFPLAPSSKSTSGGTAALGCL
VKDYFPEPVTVSWNSGALTSGVHTFPAVLQSSGLYSLSSVVTVPSSSLGT
QTYICNVNHKPSNTKVDKKVEPKSCDKTHTCPPCPAPELLGGPSVFLFPP
KPKDTLMISRTPEVTCVVVDVSHEDPEVKFNWYVDGVEVHNAKTKPREEQ
YNSTYRVVSVLTVLHQDWLNGKEYKCKVSNKALPAPIEKTISKAKGQPRE
PQVYTLPPSREEMTKNQVSLTCLVKGFYPSDIAVEWESNGQPENNYKTTP
PVLDSDGSFFLYSKLTVDKSRWQQGNVFSCSVMHEALHNHYTQKSLSLSP
GK

or the heavy-chain amino acid sequence:

(SEQ ID NO:28)

EVQLVESGGGLVQPGGSLRLSCAASGYTFTSYNMHWVRQAPGKGLEWVGA
IYPGNGDTSYNQKFKGRFTISVDKSKNTLYLQMNSLRAEDTAVYYCARVV
YYSNSYWYFDVWGQGTLVTVSSASTKGPSVFPLAPSSKSTSGGTAALGCL
VKDYFPEPVTVSWNSGALTSGVHTFPAVLQSSGLYSLSSVVTVPSSSLGT
QTYICNVNHKPSNTKVDKKVEPKSCDKTHTCPPCPAPELLGGPSVFLFPP
KPKDTLMISRTPEVTCVVVDVSHEDPEVKFNWYVDGVEVHNAKTKPREEQ
YNATYRVVSVLTVLHQDWLNGKEYKCKVSNKALPAPIAATISKAKGQPRE
PQVYTLPPSREEMTKNQVSLTCLVKGFYPSDIAVEWESNGQPENNYKTTP
PVLDSDGSFFLYSKLTVDKSRWQQGNVFSCSVMHEALHNHYTQKSLSLSP
GK.

An “isolated” antagonist is one that has been identified and separated and/or recovered from a component of its natural environment. Contaminant components of its natural environment are materials that would interfere with diagnostic or therapeutic uses for the antagonist, and may include enzymes, hormones, and other proteinaceous or non-proteinaceous solutes. In preferred embodiments, the antagonist will be purified (1) to greater than 95% by weight of antagonist as determined by the Lowry method, and most preferably more than 99% by weight, (2) to a degree sufficient to obtain at least 15 residues of N-terminal or internal amino acid sequence by use of a spinning cup sequenator, or (3) to homogeneity by SDS-PAGE under reducing or nonreducing conditions using Coomassie blue or, preferably, silver stain. Isolated antagonist includes the antagonist in situ within recombinant cells since at least one component of the antagonist's natural environment will not be present. Ordinarily, however, isolated antagonist will be prepared by at least one purification step.

“Mammal” for purposes of treatment refers to any animal classified as a mammal, including humans, domestic and farm animals, and zoo, sports, or pet animals, such as dogs, horses, cats, cows, etc. Preferably, the mammal is human.

“Treatment” refers to both therapeutic treatment and prophylactic or preventative measures. Those in need of treatment include those already with the disease or disorder as well as those in which the disease or disorder is to be prevented. Hence, the mammal may have been diagnosed as having the disease or disorder or may be predisposed or susceptible to the disease or disorder.

The expression “an effective amount” refers to an amount of the antagonist that is effective for preventing, ameliorating, or treating the autoimmune disease in question.

The term “immunosuppressive agent” as used herein for adjunct therapy refers to substances that act to suppress or mask the immune system of the mammal being treated herein. This would include substances that suppress cytokine production, downregulate or suppress self-antigen expression, or mask the MHC antigens. Examples of such agents include 2-amino-6-aryl-5-substituted pyrimidines (see U.S. Pat. No. 4,665,077); mycophenolate mofetil such as CELLCEPT®; azathioprine (IMURAN®, AZASAN®/6-mercaptopurine; bromocryptine; danazol; dapsone; glutaraldehyde (which masks the MHC antigens, as described in U.S. Pat. No. 4,120,649); anti-idiotypic antibodies for MHC antigens and MHC fragments; cyclosporin A; steroids such as corticosteroids and glucocorticosteroids, e.g., prednisone, prednisolone such as PEDIAPRED® (prednisolone sodium phosphate) or ORAPRED® (prednisolone sodium phosphate oral solution), methylprednisolone, and dexamethasone; methotrexate (oral or subcutaneous) (RHEUMATREX®, TREXALL™); hydroxycloroquine/chloroquine; sulfasalazine; leflunomide; cytokine or cytokine receptor antagonists including anti-interferon-γ, -β, or -α antibodies, anti-tumor necrosis factor-α antibodies (infliximab or adalimumab), anti-TNFα immunoadhesin (ENBREL® etanercept), anti-tumor necrosis factor-β antibodies, anti-interleukin-2 antibodies and anti-IL-2 receptor antibodies; anti-LFA-1 antibodies, including anti-CD11a and anti-CD18 antibodies; anti-L3T4 antibodies; heterologous anti-lymphocyte globulin; polyclonal or pan-T antibodies, or monoclonal anti-CD3 or anti-CD4/CD4a antibodies; soluble peptide containing a LFA-3 binding domain (WO 1990/08187 published Jul. 26, 1990); streptokinase; TGF-β; streptodornase; RNA or DNA from the host; FK506; RS-61443; deoxyspergualin; rapamycin; T-cell receptor (Cohen et al., U.S. Pat. No. 5,114,721); T-cell receptor fragments (Offner et al. Science, 251:430432 (1991); WO 1990/11294; Ianeway, Nature, 341: 482 (1989); and WO 1991/01133); T cell receptor antibodies (EP 340,109) such as T10B9; cyclophosphamide (CYTOXAN®); dapsone; penicillamine (CUPRIMINE®); plasma exchange; or intravenous immunoglobulin (IVIG). These may be used alone or in combination with each other, particularly combinations of steroid and another immunosuppressive agent or such combinations followed by a maintenance dose with a non-steroid agent to reduce the need for steroids.

“Anti-pain agent” refers to a drug that acts to inhibit or suppress pain, such as an over-the-counter analgesic or prescription pain medication to control neuralgia, such as non-steroidal anti-inflammatory drugs (NSAIDs) including ibuprofen (MOTRIN®), naproxen (NAPROSYN®), as well as various other medications used to reduce the stabbing pains that may occur, including anticonvulsants (gabapentin, phenyloin, carbamazepine) or tricyclic antidepressants. Specific examples include acetaminophen, aspirin, amitriptyline (ELAVIL®), carbamazepine (TEGRETOL®), phenyltoin (DILANTIN®), gabapentin (NEURONTIN®), (E)-N-Vanillyl-8-methyl-6-noneamid (CAPSAICIN®), or a nerve blocker.

The term “cytotoxic agent” as used herein refers to a substance that inhibits or prevents the function of cells and/or causes destruction of cells. The term is intended to include radioactive isotopes (e.g. At²¹¹, I¹³¹, I¹²⁵, Y⁹⁰, Re¹⁸⁶, Re¹⁸⁸, Sm¹⁵³, Bi²¹², P³² and radioactive isotopes of Lu), chemotherapeutic agents, and toxins such as small-molecule toxins or enzymatically active toxins of bacterial, fungal, plant or animal origin, or fragments thereof.

A “chemotherapeutic agent” is a chemical compound useful in the treatment of cancer. Examples of chemotherapeutic agents include alkylating agents such as thiotepa and CYTOXAN® cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethiylenethiophosphoramide and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially calicheamicin gamma1I and calicheamicin omegaI1 (see, e.g., Agnew, Chem Intl. Ed. Engl., 33: 183-186 (1994)); dynemicin, including dynemicin A; bisphosphonates, such as clodronate; an esperamicin; as well as neocarzinostatin chromophore and related chromoprotein enediyne antiobiotic chromophores), aclacinomysins, actinomycin, authramycin, azaserine, bleomycins, cactinomycin, carabicin, caminomycin, carzinophilin, chromomycinis, dactinomycin, daunorubicin, detorubicin, 6-diazo-5-oxo-L-norleucine, ADRIAMYCIN® doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrirnidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK® polysaccharide complex (JHS Natural Products, Eugene, Oreg.); razoxane; rhizoxin; sizofiran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., TAXOL® paclitaxel (Bristol-Myers Squibb Oncology, Princeton, N.J.), ABRAXANE™ Cremophor-free, albumin-engineered nanoparticle formulation of paclitaxel (American Pharmaceutical Partners, Schaumberg, Ill.), and TAXOTERE® doxetaxel (Rhône-Poulenc Rorer, Antony, France); chloranbucil; GEMZAR® gemcitabine; 6-thioguanine; mercaptopurine; methotrexate; platinum analogs such as cisplatin and carboplatin; vinblastine; platinum; etoposide (VP-16); ifosfamide; mitoxantrone; vincristine; NAVELBINE® vinorelbine; novantrone; teniposide; edatrexate; daunomycin; aminopterin; xeloda; ibandronate; CPT-11; topoisomerase inhibitor RFS 2000; difluorometlhylornithine (DMFO); retinoids such as retinoic acid; capecitabine; and pharmaceutically acceptable salts, acids or derivatives of any of the above.

Also included in this definition are anti-hormonal agents that act to regulate or inhibit hormone action on tumors such as anti-estrogens and selective estrogen receptor modulators (SERMs), including, for example, tamoxifen (including NOLVADEX® tamoxifen), raloxifene, droloxifene, 4-hydroxytamoxifen, trioxifene, keoxifene, LY117018, onapristone, and FARESTON toremifene; aromatase inhibitors that inhibit the enzyme aromatase, which regulates estrogen production in the adrenal glands, such as, for example, 4(5)-imidazoles, aminoglutethimide, MEGASE® megestrol acetate, AROMASIN® exemestane, formestanie, fadrozole, RIVISOR® vorozole, FEMARA® letrozole, and ARIMIDEX® anastrozole; and anti-androgens such as flutamide, nilutamide, bicalutamide, leuprolide, and goserelin; as well as troxacitabine (a 1,3-dioxolane nucleoside cytosine analog); antisense oligonucleotides, particularly those which inhibit expression of genes in signaling pathways implicated in abherant cell proliferation, such as, for example, PKC-alpha, Ralf and H-Ras; ribozymes such as a VEGF expression inhibitor (e.g., ANGIOZYME® ribozyme) and a HER2 expression inhibitor; vaccines such as gene therapy vaccines, for example, ALLOVECTIN® vaccine, LEUVECTIN® vaccine, and VAXID® vaccine; PROLEUKIN® rIL-2; LURTOTECAN® topoisomerase 1 inhibitor; ABARELIX® rmRH; and pharmaceutically acceptable salts, acids or derivatives of any of the above.

The term “cytokine” is a generic term for proteins released by one cell population that act on another cell as intercellular mediators. Examples of such cytokines are lymphokines, monokines, interleukins (ILs) such as IL-1, IL-1α, IL-2, IL-3, IL-4, IL-5, IL-6, IL-7, IL-8, IL-9, IL-11, IL-12, IL-15, a tumor necrosis factor such as TNF-α or TNF-β, and other polypeptide factors including LIF and kit ligand (KL). As used herein, the term cytokine includes proteins from natural sources or from recombinant cell culture and biologically active equivalents of the native-sequence cytokines.

The term “hormone” refers to polypeptide hormones, which are generally secreted by glandular organs with ducts. Included among the hormones are, for example, growth hormone such as human growth hormone, N-methionyl human growth hormone, and bovine growth hormone; parathyroid hormone; thyroxine; insulin; proinsulin; relaxin; prorelaxin; glycoprotein hormones such as follicle-stimulating hormone (FSH), thyroid-stimulating hormone (TSH), and luteinizing hormone (LH); prolactin; placental lactogen; mouse gonadotropin-associated peptide; inhibin; activin; mullerian-inhibiting substance; and thrombopoietin.

The term “growth factor” refers to proteins that promote growth, and includes, for example, hepatic growth factor; fibroblast growth factor; vascular endothelial growth factor; nerve growth factors such as NGF-β; platelet-derived growth factor; transforming growth factors (TGFs) such as TGF-α and TGF-β; insulin-like growth factor-I and -II; erythropoietin (EPO); osteoinductive factors; interferons such as interferon-α, -β, and -γ; and colony stimulating factors (CSFs) such as macrophage-CSF (M-CSF), granulocyte-macrophage-CSF (GM-CSF), and granulocyte-CSF (G-CSF).

The term “integrin” refers to a receptor protein that allows cells both to bind to and to respond to the extracellular matrix and is involved in a variety of cellular functions such as wound healing, cell differentiation, homing of tumor cells and apoptosis. They are part of a large family of cell-adhesion receptors that are involved in cell-extracellular matrix and cell-cell interactions. Functional integrins consist of two transmembrane glycoprotein subunits, called alpha and beta, that are non-covalently bound. The alpha subunits all share some homology to each other, as do the beta subunits. The receptors always contain one alpha chain and one beta chain. Examples include Alpha6beta1, Alpha3beta1 and Alpha7beta1.

The term “prodrug” as used in this application refers to a precursor or derivative form of a pharmaceutically active substance that is less cytotoxic to tumor cells compared to the parent drug and is capable of being enzymatically activated or converted into the more active parent form. See, e.g., Wilman, “Prodrugs in Cancer Chemotherapy” Biochemical Society Transactions, 14, pp. 375-382, 615th Meeting Belfast (1986) and Stella et al. “Prodrugs: A Chemical Approach to Targeted Drug Delivery,” Directed Drug Delivery, Borchardt et al. (ed.), pp. 247-267, Humana Press (1985). The prodrugs of this invention include, but are not limited to, phosphate-containing prodrugs, thiophosphate-containing prodrugs, sulfate-containing prodrugs, peptide-containing prodrugs, D-amino acid-modified prodrugs, glycosylated prodrugs, β-lactam-containing prodrugs, optionally substituted phenoxyacetamide-containing prodrugs or optionally substituted phenylacetamide-containing prodrugs, 5-fluorocytosine and other 5-fluorouridine prodrugs that can be converted into the more active cytotoxic free drug. Examples of cytotoxic drugs that can be derivatized into a prodrug form for use in this invention include, but are not limited to, those chemotherapeutic agents described above.

A “liposome” is a small vesicle composed of various types of lipids, phospholipids and/or surfactant that is useful for delivery of a drug (such as the antagonists disclosed herein and, optionally, a chemotherapeutic agent) to a mammal. The components of the liposome are commonly arranged in a bilayer formation, similar to the lipid arrangement of biological membranes.

The term “package insert” is used to refer to instructions customarily included in commercial packages of therapeutic products, that contain information about the indications, usage, dosage, administration, contraindications and/or warnings concerning the use of such therapeutic products.

II. Production of Antagonists

The methods and articles of manufacture of the present invention use, or incorporate, an antagonist that binds to a B-cell surface marker. Accordingly, methods for generating such antagonists will be described here.

The B-cell surface marker to be used for production of, or screening for, antagonist(s) may be, e.g., a soluble form of the antigen, or a portion thereof, containing the desired epitope. Alternatively, or additionally, cells expressing the B-cell surface marker at their cell surface can be used to generate, or screen for, antagonist(s). Other forms of the B-cell surface marker useful for generating antagonists will be apparent to those skilled in the art. Preferably, the B-cell surface marker is the CD 19 or CD20 antigen.

While the preferred antagonist is an antibody, antagonists other than antibodies are contemplated herein. For example, the antagonist may comprise a small-molecule antagonist optionally fused to, or conjugated with, a cytotoxic agent (such as those described herein). Libraries of small molecules may be screened against the B-cell surface marker of interest herein in order to identify a small molecule that binds to that antigen. The small molecule may further be screened for its antagonistic properties and/or conjugated with a cytotoxic agent.

The antagonist may also be a peptide generated by rational design or by phage display (see, e.g., WO 1998/35036 published 13 Aug. 1998). In one embodiment, the molecule of choice may be a “CDR mimic” or antibody analogue designed based on the CDRs of an antibody. While such peptides may be antagonistic by themselves, the peptide may optionally be fused to a cytotoxic agent so as to add or enhance antagonistic properties of the peptide.

A description follows as to exemplary techniques for the production of the antibody antagonists used in accordance with the present invention.

(i) Polyclonal Antibodies

Polyclonal antibodies are preferably raised in animals by multiple subcutaneous (sc) or intraperitoneal (ip) injections of the relevant antigen and an adjuvant. It may be useful to conjugate the relevant antigen to a protein that is immunogenic in the species to be immunized, e.g., keyhole limpet hemocyanin, serum albumin, bovine thyroglobulin, or soybean trypsin inhibitor, using a bifunctional or derivatizing agent, for example, maleimidobenzoyl sulfosuccinimide ester (conjugation through cysteine residues), N-hydroxysuccinimide (through lysine residues), glutaraldehyde, succinic anhydride, SOCl₂, or R¹N═C═NR, where R and R¹ are different alkyl groups.

Animals are immunized against the antigen, immunogenic conjugates, or derivatives by combining, e.g., 100 μg or 5 μg of the protein or conjugate (for rabbits or mice, respectively) with 3 volumes of Freund's complete adjuvant and injecting the solution intradermally at multiple sites. One month later the animals are boosted with ⅕ to 1/10 the original amount of peptide or conjugate in Freund's complete adjuvant by subcutaneous injection at multiple sites. Seven to 14 days later the animals are bled and the serum is assayed for antibody titer. Animals are boosted until the titer plateaus. Preferably, the animal is boosted with the conjugate of the same antigen, but conjugated to a different protein and/or through a different cross-linking reagent. Conjugates also can be made in recombinant cell culture as protein fusions. Also, aggregating agents such as alum are suitably used to enhance the immune response.

(ii) Monoclonal Antibodies

Monoclonal antibodies are obtained from a population of substantially homogeneous antibodies, i.e., the individual antibodies comprising the population are identical except for possible naturally occurring mutations that may be present in minor amounts. Thus, the modifier “monoclonal” indicates the character of the antibody as not being a mixture of discrete antibodies.

For example, the monoclonal antibodies may be made using the hybridoma method first described by Kohler et al. Nature, 256:495 (1975), or may be made by recombinant DNA methods (U.S. Pat. No. 4,816,567).

In the hybridoma method, a mouse or other appropriate host animal, such as a hamster, is immunized as hereinabove described to elicit lymphocytes that produce or are capable of producing antibodies that will specifically bind to the protein used for immunization. Alternatively, lymphocytes may be immunized in vitro. Lymphocytes then are fused with myeloma cells using a suitable fusing agent, such as polyethylene glycol, to form a hybridoma cell (Goding, Monoclonal Antibodies: Principles and Practice, pp. 59-103 (Academic Press, 1986)).

The hybridoma cells thus prepared are seeded and grown in a suitable culture medium that preferably contains one or more substances that inhibit the growth or survival of the unfused, parental myeloma cells. For example, if the parental myeloma cells lack the enzyme hypoxanthine guanine phosphoribosyl transferase (HGPRT or HPRT), the culture medium for the hybridomas typically will include hypoxanthine, aminopterin, and thymidine (HAT medium), which substances prevent the growth of HGPRT-deficient cells.

Preferred myeloma cells are those that fuse efficiently, support stable high-level production of antibody by the selected antibody-producing cells, and are sensitive to a medium such as HAT medium. Among these, preferred myeloma cell lines are murine myeloma lines, such as those derived from MOPC-21 and MPC-11 mouse tumors available from the Salk Institute Cell Distribution Center, San Diego, Calif. USA, and SP-2 or X63-Ag8-653 cells available from the American Type Culture Collection, Manassas, Va. USA. Human myeloma and mouse-human heteromyeloma cell lines also have been described for the production of human monoclonal antibodies (Kozbor, J. Immunol., 133:3001 (1984); Brodeur et al. Monoclonal Antibody Production Techniques and Applications, pp. 51-63 (Marcel Dekker, Inc., New York, 1987)).

Culture medium in which hybridoma cells are growing is assayed for production of monoclonal antibodies directed against the antigen. Preferably, the binding specificity of monoclonal antibodies produced by hybridoma cells is determined by immunoprecipitation or by an in vitro binding assay, such as radioimmunoassay (RIA) or enzyme-linked immunoabsorbent assay (ELISA).

The binding affinity of the monoclonal antibody can, for example, be determined by the Scatchard analysis of Munson et al. Anal. Biochem., 107:220 (1980).

After hybridoma cells are identified that produce antibodies of the desired specificity, affinity, and/or activity, the clones may be subcloned by limiting dilution procedures and grown by standard methods (Goding, Monoclonal Antibodies: Principles and Practice, pp. 59-103 (Academic Press, 1986)). Suitable culture media for this purpose include, for example, D-MEM or RPMI-1640 medium. In addition, the hybridoma cells may be grown in vivo as ascites tumors in an animal.

The monoclonal antibodies secreted by the subclones are suitably separated from the culture medium, ascites fluid, or serum by conventional immunoglobulin purification procedures such as, for example, protein A-SEPHAROSE™ agarose chromatography, hydroxylapatite chromatography, gel electrophoresis, dialysis, or affinity chromatography.

The monoclonal antibodies may also be produced recombinantly. DNA encoding the monoclonal antibodies is readily isolated and sequenced using conventional procedures (e.g., by using oligonucleotide probes that are capable of binding specifically to genes encoding the heavy and light chains of murine antibodies). The hybridoma cells serve as a preferred source of such DNA. Once isolated, the DNA may be placed into expression vectors, which are then transfected into host cells such as E. coli cells, simian COS cells, Chinese Hamster Ovary (CHO) cells, or myeloma cells that do not otherwise produce immunoglobulin protein, to obtain the synthesis of monoclonal antibodies in the recombinant host cells. Review articles on recombinant expression in bacteria of DNA encoding the antibody include Skerra et al. Curr. Opinion in Immunol., 5:256-262 (1993) and Plückthun, Immunol. Revs., 130:151-188 (1992).

In a further embodiment, antibodies or antibody fragments can be isolated from antibody phage libraries generated using the techniques described in McCafferty et al. Nature, 348:552-554 (1990). Clackson et al. Nature, 352:624-628 (1991) and Marks et al. J. Mol. Biol., 222:581-597 (1991) describe the isolation of murine and human antibodies, respectively, using phage libraries. Subsequent publications describe the production of high-affinity (nM range) human antibodies by chain shuffling (Marks et al. Bioffechnology, 10:779-783 (1992)), as well as combinatorial infection and in vivo recombination as a strategy for constructing very large phage libraries (Waterhouse et al. Nuc. Acids. Res., 21:2265-2266 (1993)). Thus, these techniques are viable alternatives to traditional monoclonal antibody hybridoma techniques for isolation of monoclonal antibodies.

The DNA also may be modified, for example, by substituting the coding sequence for human heavy- and light-chain constant domains in place of the homologous murine sequences (U.S. Pat. No. 4,816,567; Morrison, et al. Proc. Natl. Acad. Sci. USA, 81:6851 (1984)), or by covalently joining to the immunoglobulin coding sequence all or part of the coding sequence for a non-immunoglobulin polypeptide.

Typically such non-immunoglobulin polypeptides are substituted for the constant domains of an antibody, or they are substituted for the variable domains of one antigen-combining site of an antibody to create a chimeric bivalent antibody comprising one antigen-combining site having specificity for an antigen and another antigen-combining site having specificity for a different antigen.

(iii) Humanized Antibodies

Methods for humanizing non-human antibodies have been described in the art. Preferably, a humanized antibody has one or more amino acid residues introduced into it from a source that is non-human. These non-human amino acid residues are often referred to as “import” residues, which are typically taken from an “import” variable domain. Humanization can be essentially performed following the method of Winter and co-workers (Jones et al. Nature, 321:522-525 (1986); Riechmann et al. Nature, 332:323-327 (1988); Verhoeyen et al. Science, 239:1534-1536 (1988)), by substituting hypervariable region sequences for the corresponding sequences of a human antibody. Accordingly, such “humanized” antibodies are chimeric antibodies (U.S. Pat. No. 4,816,567) wherein substantially less than an intact human variable domain has been substituted by the corresponding sequence from a non-human species. In practice, humanized antibodies are typically human antibodies in which some hypervariable region residues and possibly some FR residues are substituted by residues from analogous sites in rodent antibodies.

The choice of human variable domains, both light and heavy, to be used in making the humanized antibodies is very important to reduce antigenicity. According to the so-called “best-fit” method, the sequence of the variable domain of a rodent antibody is screened against the entire library of known human variable-domain sequences. The human sequence that is closest to that of the rodent is then accepted as the human framework region (FR) for the humanized antibody (Sims et al. J. Immunol., 151:2296 (1993); Chothia et al. J. Mol. Biol., 196:901 (1987)). Another method uses a particular framework region derived from the consensus sequence of all human antibodies of a particular subgroup of light or heavy chains. The same framework may be used for several different humanized antibodies (Carter et al. Proc. Natl. Acad. Sci. USA, 89:4285 (1992); Presta et al. J. Immunol., 151:2623 (1993)).

It is further important that antibodies be humanized with retention of high affinity for the antigen and other favorable biological properties. To achieve this goal, according to a preferred method, humanized antibodies are prepared by a process of analysis of the parental sequences and various conceptual humanized products using three-dimensional models of the parental and humanized sequences. Three-dimensional immunoglobulin models are commonly available and are familiar to those skilled in the art. Computer programs are available that illustrate and display probable three-dimensional conformational structures of selected candidate immunoglobulin sequences. Inspection of these displays permits analysis of the likely role of the residues in the functioning of the candidate immunoglobulin sequence, i.e., the analysis of residues that influence the ability of the candidate immunoglobulin to bind its antigen. In this way, FR residues can be selected and combined from the recipient and import sequences so that the desired antibody characteristic, such as increased affinity for the target antigen(s), is achieved. In general, the hypervariable region residues are directly and most substantially involved in influencing antigen binding.

(iv) Human Antibodies

As an alternative to humanization, human antibodies can be generated. For example, it is now possible to produce transgenic animals (e.g., mice) that are capable, upon immunization, of producing a full repertoire of human antibodies in the absence of endogenous immunoglobulin production. For example, it has been described that the homozygous deletion of the antibody heavy-chain joining region (J_{H}) gene in chimeric and germ-line mutant mice results in complete inhibition of endogenous antibody production. Transfer of the human germ-line immunoglobulin gene array in such germ-line mutant mice will result in the production of human antibodies upon antigen challenge. See, e.g., Jakobovits et al. Proc. Natl. Acad. Sci. USA, 90:2551 (1993); Jakobovits et al. Nature, 362:255-258 (1993); Bruggermann et al. Year in Immuno., 7:33 (1993); and U.S. Pat. Nos. 5,591,669, 5,589,369 and 5,545,807.

Alternatively, phage-display technology (McCafferty et al. Nature 348:552-553 (1990)) can be used to produce human antibodies and antibody fragments in vitro, from immunoglobulin variable (V) domain gene repertoires from unimmunized donors. According to this technique, antibody V domain genes are cloned in-frame into either a major or minor coat protein gene of a filamentous bacteriophage, such as M13 or fd, and displayed as functional antibody fragments on the surface of the phage particle. Because the filamentous particle contains a single-stranded DNA copy of the phage genome, selections based on the functional properties of the antibody also result in selection of the gene encoding the antibody exhibiting those properties. Thus, the phage mimics some of the properties of the B cell. Phage display can be performed in a variety of formats; for their review see, e.g., Johnson, Kevin S. and Chiswell, David J., Current Opinion in Structural Biology 3:564-571 (1993). Several sources of V-gene segments can be used for phage display. Clackson et al. Nature, 352:624-628 (1991) isolated a diverse array of anti-oxazolone antibodies from a small random combinatorial library of V genes derived from the spleens of immunized mice. A repertoire of V genes from unimmunized human donors can be constructed and antibodies to a diverse array of antigens (including self-antigens) can be isolated essentially following the techniques described by Marks et al. J. Mol. Biol. 222:581-597 (1991), or Griffith et al. EMBO J. 12:725-734 (1993). See, also, U.S. Pat. Nos. 5,565,332 and 5,573,905.

Human antibodies may also be generated by in vitro activated B cells (see U.S. Pat. Nos. 5,567,610 and 5,229,275).

(v) Antibody Fragments

Various techniques have been developed for the production of antibody fragments. Traditionally, these fragments were derived via proteolytic digestion of intact antibodies (see, e.g., Morimoto et al. Journal of Biochemical and Biophysical Methods 24:107-117 (1992) and Brennan et al. Science, 229:81 (1985)). However, these fragments can now be produced directly by recombinant host cells. For example, the antibody fragments can be isolated from the antibody phage libraries discussed above. Alternatively, Fab′-SH fragments can be directly recovered from E. coli and chemically coupled to form F(ab′)₂ fragments (Carter et al. Bio/Technology 10:163-167 (1992)). According to another approach, F(ab′)₂ fragments can be isolated directly from recombinant host cell culture. Other techniques for the production of antibody fragments will be apparent to the skilled practitioner. In other embodiments, the antibody of choice is a single-chain Fv fragment (scFv). See WO 1993/16185; U.S. Pat. No. 5,571,894; and U.S. Pat. No. 5,587,458. The antibody fragment may also be a “linear antibody,” e.g., as described in U.S. Pat. No. 5,641,870, for example. Such linear antibody fragments may be monospecific or bispecific.

(vi) Bispecific Antibodies

Bispecific antibodies are antibodies that have binding specificities for at least two different epitopes. Exemplary bispecific antibodies may bind to two different epitopes of the B-cell surface marker. Other such antibodies may bind a first B-cell surface marker and further bind a second B-cell surface marker. Alternatively, an anti-B-cell surface marker binding arm may be combined with an arm that binds to a triggering molecule on a leukocyte such as a T-cell receptor molecule (e.g. CD2 or CD3), or Fc receptors for IgG (FcγR), such as FcγRI (CD64), FcγRII (CD32), and FcγRIII (CD 16), so as to focus cellular defense mechanisms to the B cell. Bispecific antibodies may also be used to localize cytotoxic agents to the B cell. These antibodies possess a B-cell surface marker-binding arm and an arm that binds the cytotoxic agent (e.g. saporin, anti-interferon-α, vinca alkaloid, ricin A chain, methotrexate, or radioactive isotope hapten). Bispecific antibodies can be prepared as full-length antibodies or antibody fragments (e.g. F(ab′)₂ bispecific antibodies).

Methods for making bispecific antibodies are known in the art. Traditional production of full-length bispecific antibodies is based on the co-expression of two immunoglobulin heavy-chain-light-chain pairs, where the two chains have different specificities (Millstein et al. Nature, 305:537-539 (1983)). Because of the random assortment of immunoglobulin heavy and light chains, these hybridomas (quadromas) produce a potential mixture of 10 different antibody molecules, of which only one has the correct bispecific structure. Purification of the correct molecule, which is usually done by affinity chromatography steps, is rather cumbersome, and the product yields are low. Similar procedures are disclosed in WO 1993/08829, and in Traunecker et al. EMBO J., 10:3655-3659 (1991).

According to a different approach, antibody variable domains with the desired binding specificities (antibody-antigen combining sites) are fused to immunoglobulin constant domain sequences. The fusion preferably is with an immunoglobulin heavy-chain constant domain, comprising at least part of the hinge, CH2, and CH3 regions. It is preferred to have the first heavy-chain constant region (CH1), containing the site necessary for light-chain binding, present in at least one of the fusions. DNAs encoding the immunoglobulin heavy-chain fusions and, if desired, the immunoglobulin light chain, are inserted into separate expression vectors, and are co-transfected into a suitable host organism. This provides for great flexibility in adjusting the mutual proportions of the three polypeptide fragments in embodiments when unequal ratios of the three polypeptide chains used in the construction provide the optimum yields. It is, however, possible to insert the coding sequences for two or all three polypeptide chains in one expression vector when the expression of at least two polypeptide chains in equal ratios results in high yields or when the ratios are of no particular significance.

In a preferred embodiment of this approach, the bispecific antibodies are composed of a hybrid immunoglobulin heavy chain with a first binding specificity in one arm, and a hybrid immunoglobulin heavy-chain-light-chain pair (providing a second binding specificity) in the other arm. It was found that this asymmetric structure facilitates the separation of the desired bispecific compound from unwanted immunoglobulin chain combinations, as the presence of an immunoglobulin light chain in only one half of the bispecific molecule provides for a facile way of separation. This approach is disclosed in WO 1994/04690. For further details of generating bispecific antibodies, see, for example, Suresh et al. Methods in Enzymology, 121:210 (1986).

According to another approach described in U.S. Pat. No. 5,731,168, the interface between a pair of antibody molecules can be engineered to maximize the percentage of heterodimers that are recovered from recombinant cell culture. The preferred interface comprises at least a part of the C_{H}3 domain of an antibody constant domain. In this method, one or more small amino acid side chains from the interface of the first antibody molecule are replaced with larger side chains (e.g. tyrosine or tryptophan). Compensatory “cavities” of identical or similar size to the large side chain(s) are created on the interface of the second antibody molecule by replacing large amino acid side chains with smaller ones (e.g. alanine or threonine). This provides a mechanism for increasing the yield of the heterodimer over other unwanted end-products such as homodimers.

Bispecific antibodies include cross-linked or “heteroconjugate” antibodies. For example, one of the antibodies in the heteroconjugate can be coupled to avidin, the other to biotin. Such antibodies have, for example, been proposed to target immune system cells to unwanted cells (U.S. Pat. No. 4,676,980), and for treatment of HIV infection (WO 1991/00360, WO 1992/200373, and EP 03089). Heteroconjugate antibodies may be made using any convenient cross-linking methods. Suitable cross-linking agents are well known in the art, and are disclosed, for example, in U.S. Pat. No. 4,676,980, along with a number of cross-linking techniques.

Techniques for generating bispecific antibodies from antibody fragments have also been described in the literature. For example, bispecific antibodies can be prepared using chemical linkage. Brennan et al. Science, 229: 81 (1985) describe a procedure wherein intact antibodies are proteolytically cleaved to generate F(ab′)₂ fragments. These fragments are reduced in the presence of the dithiol complexing agent sodium arsenite to stabilize vicinal dithiols and prevent intermolecular disulfide formation. The Fab′ fragments generated are then converted to thionitrobenzoate (TNB) derivatives. One of the Fab′-TNB derivatives is then reconverted to the Fab′-thiol by reduction with mercaptoethylamine and is mixed with an equimolar amount of the other Fab′-TNB derivative to form the bispecific antibody. The bispecific antibodies produced can be used as agents for the selective immobilization of enzymes.

Recent progress has facilitated the direct recovery of Fab′-SH fragments from E. coli, which can be chemically coupled to form bispecific antibodies. Shalaby et al. J. Exp. Med., 175:217-225 (1992) describe the production of a fully humanized bispecific antibody F(ab′)₂ molecule. Each Fab′ fragment was separately secreted from E. coli and subjected to directed chemical coupling in vitro to form the bispecific antibody. The bispecific antibody thus formed was able to bind to cells overexpressing the ErbB2 receptor and normal human T cells, as well as trigger the lytic activity of human cytotoxic lymphocytes against human breast tumor targets.

Various techniques for making and isolating bispecific antibody fragments directly from recombinant cell culture have also been described. For example, bispecific antibodies have been produced using leucine zippers. Kostelny et al. J. Immunol., 148(5):1547-1553 (1992). The leucine zipper peptides from the Fos and Jun proteins were linked to the Fab′ portions of two different antibodies by gene fusion. The antibody homodimers were reduced at the hinge region to form monomers and then re-oxidized to form the antibody heterodimers. This method can also be utilized for the production of antibody homodimers. The “diabody” technology described by Hollinger et al. Proc. Natl. Acad. Sci. USA, 90:6444-6448 (1993) has provided an alternative mechanism for making bispecific antibody fragments. The fragments comprise a heavy-chain variable domain (V_{H}) connected to a light-chain variable domain (V_{L}) by a linker that is too short to allow pairing between the two domains on the same chain. Accordingly, the V_{H} and V_{L} domains of one fragment are forced to pair with the complementary V_{L} and V_{H} domains of another fragment, thereby forming two antigen-binding sites. Another strategy for making bispecific antibody fragments by the use of single-chain Fv (sFv) dimers has also been reported. See Gruber et al. J. Immunol., 152:5368 (1994).

Antibodies with more than two valencies are contemplated. For example, trispecific antibodies can be prepared. Tutt et al. J. Immunol. 147: 60 (1991).

III. Conjugates and Other Modifications of the Antagonist

The antagonist used in the methods or included in the articles of manufacture herein is optionally conjugated to a cytotoxic agent.

Chemotherapeutic agents useful in the generation of such antagonist-cytotoxic agent conjugates have been described above.

Conjugates of an antagonist and one or more small-molecule toxins, such as a calicheamicin, a maytansine (U.S. Pat. No. 5,208,020), a trichothene, and CC1065, are also contemplated herein. In one embodiment of the invention, the antagonist is conjugated to one or more maytansine molecules (e.g. about 1 to about 10 maytansine molecules per antagonist molecule). Maytansine may, for example, be converted to May-SS-Me, which may be reduced to May-SH3 and reacted with modified antagonist (Chari et al. Cancer Research 52: 127-131 (1992)) to generate a maytansinoid-antagonist conjugate.

Alternatively, the antagonist is conjugated to one or more calicheamicin molecules. The calicheamicin family of antibiotics is capable of producing double-stranded DNA breaks at sub-picomolar concentrations. Structural analogues of calicheamicin that may be used include, but are not limited to, γ₁^{I}, α₂^{I}, α₃^{I}, N-acetyl-γ1^{I}, PSAG and θ^{I}₁ (Hinman et al. Cancer Research 53: 3336-3342 (1993) and Lode et al. Cancer Research 58: 2925-2928 (1998)).

Enzymatically active toxins and fragments thereof that can be used include diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), momordica charantia inhibitor, curcin, crotin, sapaonaria officinalis inhibitor, gelonin, mitogellin, restrictocin, phenomycin, enomycin and the tricothecenes. See, for example, WO 1993/21232 published Oct. 28, 1993.

The present invention further contemplates antagonist conjugated with a compound with nucleolytic activity (e.g. a ribonuclease or a DNA endonuclease such as a deoxyribonuclease; DNase).

A variety of radioactive isotopes are available for the production of radioconjugated antagonists. Examples include At²¹¹, I¹³¹, I¹²⁵, Y⁹⁰, Re¹⁸⁶, Re¹⁸⁸ Sm¹⁵³, Bi²¹², P³² and radioactive isotopes of Lu.

Conjugates of the antagonist and cytotoxic agent may be made using a variety of bifunctional protein coupling agents such as N-succinimidyl-3-(2-pyridyldithiol) propionate (SPDP), succinimidyl-4-(N-maleimidomethyl) cyclohexane-1-carboxylate, iminothiolane (IT), bifunctional derivatives of imidoesters (such as dimethyl adipimidate HCL), active esters (such as disuccinimidyl suberate), aldehydes (such as glutareldehyde), bis-azido compounds (such as bis (p-azidobenzoyl) hexanediamine), bis-diazonium derivatives (such as bis-(p-diazoniumbenzoyl)-ethylenediamine), diisocyanates (such as tolyene 2,6-diisocyanate), and bis-active fluorine compounds (such as 1,5-difluoro-2,4-dinitrobenzene). For example, a ricin immunotoxin can be prepared as described in Vitetta et al. Science 238: 1098 (1987). Carbon-14-labeled 1-isothiocyanatobenzyl-3-methyldiethylene triaminepentaacetic acid (MX-DTPA) is an exemplary chelating agent for conjugation of radionucleotide to the antagonist. See WO 1994/11026. The linker may be a “cleavable linker” facilitating release of the cytotoxic drug in the cell. For example, an acid-labile linker, peptidase-sensitive linker, dimethyl linker or disulfide-containing linker (Chari et al. Cancer Research 52: 127-131 (1992)) may be used.

Alternatively, a fusion protein comprising the antagonist and cytotoxic agent may be made, e.g., by recombinant techniques or peptide synthesis.

In yet another embodiment, the antagonist may be conjugated to a “receptor” (such as streptavidin) for utilization in tumor pretargeting wherein the antagonist-receptor conjugate is administered to the patient, followed by removal of unbound conjugate from the circulation using a clearing agent and then administration of a “ligand” (e.g. avidin) that is conjugated to a cytotoxic agent (e.g. a radionucleotide).

The antagonists of the present invention may also be conjugated with a prodrug-activating enzyme that converts a prodrug (e.g., a peptidyl chemotherapeutic agent, see WO 1981/01145) to an active anti-cancer drug. See, for example, WO 1988/07378 and U.S. Pat. No. 4,975,278.

The enzyme component of such conjugates includes any enzyme capable of acting on a prodrug in such a way so as to convert it into its more active, cytotoxic form.

Enzymes that are useful in the method of this invention include, but are not limited to, alkaline phosphatase useful for converting phosphate-containing prodrugs into free drugs; arylsulfatase useful for converting sulfate-containing prodrugs into free drugs; cytosine deaminase useful for converting non-toxic 5-fluorocytosine into the anti-cancer drug, 5-fluorouracil; proteases, such as serratia protease, thermolysin, subtilisin, carboxypeptidases and cathepsins (such as cathepsins B and L), that are useful for converting peptide-containing prodrugs into free drugs; D-alanylcarboxypeptidases, useful for converting prodrugs that contain D-amino acid substituents; carbohydrate-cleaving enzymes such as β-galactosidase and neuraminidase useful for converting glycosylated prodrugs into free drugs; β-lactamase useful for converting drugs derivatized with β-lactams into free drugs; and penicillin amidases, such as penicillin V amidase or penicillin G amidase, useful for converting drugs derivatized at their amine nitrogens with phenoxyacetyl or phenylacetyl groups, respectively, into free drugs. Alternatively, antibodies with enzymatic activity, also known in the art as “abzymes”, can be used to convert the prodrugs of the invention into free active drugs (see, e.g., Massey, Nature 328: 457-458 (1987)). Antagonist-abzyme conjugates can be prepared as described herein for delivery of the abzyme to a tumor cell population.

The enzymes of this invention can be covalently bound to the antagonist by techniques well known in the art such as the use of the heterobifunctional crosslinking reagents discussed above. Alternatively, fusion proteins comprising at least the antigen-binding region of an antagonist of the invention linked to at least a functionally active portion of an enzyme of the invention can be constructed using recombinant DNA techniques well known in the art (see, e.g., Neuberger et al. Nature, 312:604-608 (1984)).

Other modifications of the antagonist are contemplated herein. For example, the antagonist may be linked to one of a variety of nonproteinaceous polymers, e.g., polyethylene glycol, polypropylene glycol, polyoxyalkylenes, or copolymers of polyethylene glycol and polypropylene glycol.

The antagonists disclosed herein may also be formulated as liposomes. Liposomes containing the antagonist are prepared by methods known in the art, such as described in Epstein et al. Proc. Natl. Acad. Sci. USA, 82:3688 (1985); Hwang et al. Proc. Natl. Acad. Sci. USA, 77:4030 (1980); U.S. Pat. Nos. 4,485,045 and 4,544,545; and WO 1997/38731 published Oct. 23, 1997. Liposomes with enhanced circulation time are disclosed in U.S. Pat. No. 5,013,556.

Particularly useful liposomes can be generated by the reverse-phase evaporation method with a lipid composition comprising phosphatidylcholine, cholesterol, and PEG-derivatized phosphatidylethanolamine (PEG-PE). Liposomes are extruded through filters of defined pore size to yield liposomes with the desired diameter. Fab′ fragments of an antibody of the present invention can be conjugated to the liposomes as described in Martin et al. J. Biol. Chem. 257: 286-288 (1982) via a disulfide-interchange reaction. A chemotherapeutic agent is optionally contained within the liposome. See Gabizon et al. J. National Cancer Inst. 81(19):1484 (1989).

Amino acid sequence modification(s) of protein or peptide antagonists described herein are contemplated. For example, it may be desirable to improve the binding affinity and/or other biological properties of the antagonist. Amino acid sequence variants of the antagonist are prepared by introducing appropriate nucleotide changes into the antagonist nucleic acid, or by peptide synthesis. Such modifications include, for example, deletions from, and/or insertions into and/or substitutions of, residues within the amino acid sequences of the antagonist. Any combination of deletion, insertion, and substitution is made to arrive at the final construct, provided that the final construct possesses the desired characteristics. The amino acid changes also may alter post-translational processes of the antagonist, such as changing the number or position of glycosylation sites.

A useful method for identification of certain residues or regions of the antagonist that are preferred locations for mutagenesis is called “alanine-scanning mutagenesis” as described by Cunningham and Wells Science, 244:1081-1085 (1989). Here, a residue or group of target residues are identified (e.g., charged residues such as arg, asp, his, lys, and glu) and replaced by a neutral or negatively charged amino acid (most preferably alanine or polyalanine) to affect the interaction of the amino acids with antigen. Those amino acid locations demonstrating functional sensitivity to the substitutions then are refined by introducing further or other variants at, or for, the sites of substitution. Thus, while the site for introducing an amino acid sequence variation is predetermined, the nature of the mutation per se need not be predetermined. For example, to analyze the performance of a mutation at a given site, ala scanning or random mutagenesis is conducted at the target codon or region and the expressed antagonist variants are screened for the desired activity.

Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include an antagonist with an N-terminal methionyl residue or the antagonist fused to a cytotoxic polypeptide. Other insertional variants of the antagonist molecule include the fusion to the N- or C-terminus of the antagonist of an enzyme, or a polypeptide that increases the serum half-life of the antagonist.

Another type of variant is an amino acid substitution variant. These variants have at least one amino acid residue in the antagonist molecule replaced by a different residue. The sites of greatest interest for substitutional mutagenesis of antibody antagonists include the hypervariable regions, but FR alterations are also contemplated. Conservative substitutions are shown in Table I under the heading of “preferred substitutions”. If such substitutions result in a change in biological activity, then more substantial changes, denominated “exemplary substitutions” in Table 1, or as further described below in reference to amino acid classes, may be introduced and the products screened.

TABLE 1

| Original | Exemplary | Preferred |
|---|---|---|
| Residue | Substitutions | Substitutions |
| Ala (A) | val; leu; ile | val |
| Arg (R) | lys; gln; asn | lys |
| Asn (N) | gln; his; asp, lys; arg | gln |
| Asp (D) | glu; asn | glu |
| Cys (C) | ser; ala | ser |
| Gln (Q) | asn; glu | asn |
| Glu (E) | asp; gln | asp |
| Gly (G) | ala | ala |
| His (H) | asn; gln; lys; arg | arg |
| Ile (I) | leu; val; met; ala; phe; norleucine | leu |
| Leu (L) | norleucine; ile; val; met; ala; phe | ile |
| Lys (K) | arg; gln; asn | arg |
| Met (M) | leu; phe; ile | leu |
| Phe (F) | leu; val; ile; ala; tyr | tyr |
| Pro (P) | ala | ala |
| Ser (S) | thr | thr |
| Thr (T) | ser | ser |
| Trp (W) | tyr; phe | tyr |
| Tyr (Y) | trp; phe; thr; ser | phe |
| Val (V) | ile; leu; met; phe; ala; norleucine | leu |

Substantial modifications in the biological properties of the antagonist are accomplished by selecting substitutions that differ significantly in their effect on maintaining (a) the structure of the polypeptide backbone in the area of the substitution, for example, as a sheet or helical conformation, (b) the charge or hydrophobicity of the molecule at the target site, or (c) the bulk of the side chain. Naturally occurring residues are divided into groups based on common side-chain properties:
(1) hydrophobic: norleucine, met, ala, val, leu, ile;
(2) neutral hydrophilic: cys, ser, thr;
(3) acidic: asp, glu;
(4) basic: asn, gln, his, lys, arg;
(5) residues that influence chain orientation: gly, pro; and
(6) aromatic: trp, tyr, phe.

Non-conservative substitutions will entail exchanging a member of one of these classes for another class.

Any cysteine residue not involved in maintaining the proper conformation of the antagonist also may be substituted, generally with serine, to improve the oxidative stability of the molecule and prevent aberrant crosslinking. Conversely, cysteine bond(s) may be added to the antagonist to improve its stability (particularly where the antagonist is an antibody fragment such as an Fv fragment).

A particularly preferred type of substitutional variant involves substituting one or more hypervariable region residues of a parent antibody. Generally, the resulting variant(s) selected for further development will have improved biological properties relative to the parent antibody from which they are generated. A convenient way for generating such substitutional variants is affinity maturation using phage display. Briefly, several hypervariable region sites (e.g. 6-7 sites) are mutated to generate all possible amino acid substitutions at each site. The antibody variants thus generated are displayed in a monovalent fashion from filamentous phage particles as fusions to the gene III product of M13 packaged within each particle. The phage-displayed variants are then screened for their biological activity (e.g. binding affinity) as herein disclosed. In order to identify candidate hypervariable region sites for modification, alanine-scanning mutagenesis can be performed to identify hypervariable region residues contributing significantly to antigen binding. Alternatively, or additionally, it may be beneficial to analyze a crystal structure of the antigen-antibody complex to identify contact points between the antibody and antigen. Such contact residues and neighboring residues are candidates for substitution according to the techniques elaborated herein. Once such variants are generated, the panel of variants is subjected to screening as described herein and antibodies with superior properties in one or more relevant assays may be selected for further development.

Another type of amino acid variant of the antagonist alters the original glycosylation pattern of the antagonist. By altering is meant deleting one or more carbohydrate moieties found in the antagonist, and/or adding one or more glycosylation sites that are not present in the antagonist.

Glycosylation of polypeptides is typically either N-linked or O-linked. N-linked refers to the attachment of the carbohydrate moiety to the side chain of an asparagine residue. The tripeptide sequences asparagine-X-serine and asparagine-X-threonine, where X is any amino acid except proline, are the recognition sequences for enzymatic attachment of the carbohydrate moiety to the asparagine side chain. Thus, the presence of either of these tripeptide sequences in a polypeptide creates a potential glycosylation site. O-linked glycosylation refers to the attachment of one of the sugars N-aceylgalactosamine, galactose, or xylose to a hydroxyamino acid, most commonly serine or threonine, although 5-hydroxyproline or 5-hydroxylysine may also be used.

Addition of glycosylation sites to the antagonist is conveniently accomplished by altering the amino acid sequence such that it contains one or more of the above-described tripeptide sequences (for N-linked glycosylation sites). The alteration may also be made by the addition of, or substitution by, one or more serine or threonine residues to the sequence of the original antagonist (for O-linked glycosylation sites).

Nucleic acid molecules encoding amino acid sequence variants of the antagonist are prepared by a variety of methods known in the art. These methods include, but are not limited to, isolation from a natural source (in the case of naturally occurring amino acid sequence variants) or preparation by oligonucleotide-mediated (or site-directed) mutagenesis, PCR mutagenesis, and cassette mutagenesis of an earlier prepared variant or a non-variant version of the antagonist.

It may be desirable to modify the antagonist of the invention with respect to effector function, e.g. so as to enhance ADCC and/or CDC of the antagonist. This may be achieved by introducing one or more amino acid substitutions in an Fc region of an antibody antagonist. Alternatively or additionally, cysteine residue(s) may be introduced in the Fc region, thereby allowing interchain disulfide bond formation in this region. The homodimeric antibody thus generated may have improved internalization capability and/or increased complement-mediated cell killing and ADCC. See Caron et al. J. Exp Med. 176:1191-1195 (1992) and Shopes, B. J. Immunol. 148:2918-2922 (1992). Homodimeric antibodies with enhanced anti-tumor activity may also be prepared using heterobifunctional cross-linkers as described in Wolff et al. Cancer Research 53:2560-2565 (1993). Alternatively, an antibody can be engineered that has dual Fc regions and may thereby have enhanced complement lysis and ADCC capabilities. See Stevenson et al. Anti-Cancer Drug Design 3:219-230 (1989).

To increase the serum half-life of the antagonist, one may incorporate a salvage receptor binding epitope into the antagonist (especially an antibody fragment) as described in U.S. Pat. No. 5,739,277, for example. As used herein, the term “salvage receptor binding epitope” refers to an epitope of the Fc region of an IgG molecule (e.g., IgG₁, IgG₂, IgG₃, or IgG₄) that is responsible for increasing the in vivo serum half-life of the IgG molecule.

IV. Pharmaceutical Formulations

Therapeutic formulations of the antagonists used in accordance with the present invention are prepared for storage by mixing an antagonist having the desired degree of purity with optional pharmaceutically acceptable carriers, excipients or stabilizers (Remington's Pharmaceutical Sciences 16th edition, Osol, A. Ed. (1980)), in the form of lyophilized formulations or aqueous solutions. Acceptable carriers, excipients, or stabilizers are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low-molecular-weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as TWEEN®, PLURONICS®, or polyethylene glycol (PEG).

Exemplary anti-CD20 antibody formulations are described in WO 1998/56418. This publication describes a liquid multidose formulation comprising 40 mg/mL rituximab, 25 mM acetate, 150 mM trehalose, 0.9% benzyl alcohol, and 0.02% POLYSORBATE™ 20 (polyoxyethylene sorbitan monooleate) at pH 5.0 that has a minimum shelf life of two years storage at 2-8° C. Another anti-CD20 formulation of interest comprises 10 mg/mL rituximab in 9.0 mg/mL sodium chloride, 7.35 mg/mL sodium citrate dihydrate, 0.7 mg/mL POLYSORBATE™ 80 (polyoxyethylene sorbitan monooleate), and Sterile Water for Injection, pH 6.5.

Lyophilized formulations adapted for subcutaneous administration are described in WO 1997/04801. Such lyophilized formulations may be reconstituted with a suitable diluent to a high protein concentration and the reconstituted formulation may be administered subcutaneously to the mammal to be treated herein.

The formulation herein may also contain more than one active compound as necessary for the particular indication being treated, preferably those with complementary activities that do not adversely affect each other. For example, it may be desirable to further provide a cytotoxic agent, chemotherapeutic agent, cytokine, or immunosuppressive agent (e.g. one that acts on T cells, such as cyclosporin or an antibody that binds T cells, e.g,. one that binds LFA-1). The effective amount of such other agents depends on the amount of antagonist present in the formulation, the type of disease or disorder or treatment, and other factors discussed above. These are generally used in the same dosages and with administration routes as used hereinbefore or about from 1 to 99% of the heretofore employed dosages.

The active ingredients may also be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 16th edition, Osol, A. Ed. (1980).

Sustained-release preparations may be prepared. Suitable examples of sustained-release preparations include semipermeable matrices of solid hydrophobic polymers containing the antagonist, which matrices are in the form of shaped articles, e.g. films, or microcapsules. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and γ ethyl-L-glutamate, non-degradable ethylene-vinyl acetate, degradable lactic acid-glycolic acid copolymers such as the LUPRON DEPOT® (injectable microspheres composed of lactic acid-glycolic acid copolymer and leuprolide acetate), and poly-D-(−)-3-hydroxybutyric acid.

The formulations to be used for in vivo administration must be sterile. This is readily accomplished by filtration through sterile filtration membranes.

V. Treatment with the Antagonist

The composition comprising an antagonist that binds to a B-cell surface antigen will be formulated, dosed, and administered in a fashion consistent with good medical practice. Factors for consideration in this context include the particular disease or disorder being treated, the particular mammal being treated, the clinical condition of the individual patient, the cause of the disease or disorder, the site of delivery of the agent, the method of administration, the scheduling of administration, and other factors known to medical practitioners. The therapeutically effective amount of the antagonist to be administered will be governed by such considerations.

As a general proposition, the therapeutically effective amount of the antagonist administered parenterally per dose will be in the range of about 0.1 to 20 mg/kg of patient body weight per day, with the typical initial range of antagonist used being in the range of about 2 to 10 mg/kg.

The preferred antagonist is an antibody, e.g. an antibody such as RITUXAN®, which is not conjugated to a cytotoxic agent. Suitable dosages for an unconjugated antibody are, for example, in the range from about 20 mg/m² to about 1000 mg/m². In one embodiment, the dosage of the antibody differs from that presently recommended for RITUXAN®. For example, one may administer to the patient one or more doses of substantially less than 375 mg/m² of the antibody, e.g. where the dose is in the range from about 20 mg/m² to about 250 mg/m², for example, from about 50 mg/m² to about 200 mg/m².

Moreover, one may administer one or more initial dose(s) of the antibody followed by one or more subsequent dose(s), wherein the mg/m² dose of the antibody in the subsequent dose(s) exceeds the mg/m² dose of the antibody in the initial dose(s). For example, the initial dose may be in the range from about 20 mg/m to about 250 mg/m² (e.g., from about 50 mg/m² to about 200 mg/m²) and the subsequent dose may be in the range from about 250 mg/m² to about 1000 mg/m².

As noted above, however, these suggested amounts of antagonist are subject to a great deal of therapeutic discretion. The key factor in selecting an appropriate dose and scheduling is the result obtained, as indicated above. For example, relatively higher doses may be needed initially for the treatment of ongoing and acute diseases. To obtain the most efficacious results, depending on the disease or disorder, the antagonist is administered as close to the first sign, diagnosis, appearance, or occurrence of the disease or disorder as possible or during remissions of the disease or disorder.

The antagonist is administered by any suitable means, including parenteral, subcutaneous, intra-peritoneal, inhalational, intra-thecal, intra-articular, and intra-nasal, and, if desired for local immunosuppressive treatment, intralesional administration. Parenteral infusions include intramuscular, intravenous, intra-arterial, intraperitoneal, or subcutaneous administration. In addition, the antagonist may suitably be administered by pulse infusion, e.g., with declining doses of the antagonist. Preferably the dosing is given by injections, most preferably intravenous or subcutaneous injections, depending in part on whether the administration is brief or chronic.

One may administer one or more other compounds, such as cytotoxic agents, chemotherapeutic agents, immunosuppressive agents, anti-pain agents, hormones, integrins, growth factors, and/or cytokines with the antagonists herein, or apply various other therapies known to those skilled in the art. Preferably, depending, for example, on the type of indication, the degree or severity of the indication, and the type of antagonist, the other compound administered is an immunosuppressive agent, an anti-pain agent, or a chemotherapeutic agent.

If polychondritis is treated such as relapsing polychondritis, preferably the other compound, if the symptoms are not severe, is a non-steroidal anti-inflammatory drug (NSAID), including ibuprofen (MOTRIN®), naproxen (NAPROSYN®), or sulindac (CLINORIL®), to control the inflammation. Usually, however, cortisone-related medications are required, e.g., steroids such as prednisone and prednisolone. High-dose steroids are frequently necessary initially, especially when the eyes or breathing airways are involved. Moreover, most patients require steroids for long-term use.

Another preferred compound that can be used in combination with the antagonist for treating polychondritis is methotrexate (RHEUMATREX®, TREXALL™), which has shown promise as a treatment for relapsing polychondritis in combination with steroids as well as a maintenance treatment. Studies have demonstrated that methotrexate can help reduce the steroid requirements. Other preferred compounds include cyclophosphamide (CYTOXAN®), dapsone, azathioprine (IMURAN®, AZASAN®), penicillamine (CUPRIMINE®), cyclosporine (NEORAL®, SANDIMMUNE®), and combinations of these drugs with steroids.

Regarding treatment of mononeuritis multiplex with another agent, if a specific treatment is not available, the pain of the neuropathy can usually be controlled. The simplest treatment is an over-the-counter analgesic, such as acetaminophen (TYLENOL®), a NSAID such as ibuprofen as noted above, or aspirin, followed by a prescription pain medication. Tricyclic antidepressants such as amitriptyline (ELAVIL®) and anti-seizure medications, such as carbamazepine (TEGRETOL®), phenyltoin (DILANTIN®), or gabapentin (NEURONTIN®), have been used to relieve the pain of neuropathy. CAPSAICIN® ((E)-N-Vanillyl-8-methyl-6-noneamid), the chemical responsible for chili peppers being hot, is used as a cream to help relieve the pain of a peripheral neuropathy. Additionally, a nerve blocker may be effective at relieving the pain. Other preferred compounds for treatment of peripheral neuropathies include autologous PBSC transplantation, steroids such as corticosteroids including pulse therapy thereof and prednisone, prednisolone, and methyl-prednisolone including pulse therapy thereof, methotrexate, cyclophosphamide (e.g., CYTOXAN®) including intravenous cyclophosphamide pulse therapy, plasma exchange or plasmapheresis, intravenous immunoglobulin, cyclosporines such as cyclosporin A, mycophenolate mofetil (e.g., CELLCEPT®), or chemotherapeutic agents (including high doses thereof) including those that lower IgM concentrations, such as FLUDARA® (fludarabine phosphate) or LEUKERAN® (chlorambucil). Particularly preferred other compounds for this indication are anti-pain agents, steroids, methotrexate, cyclophosphamide, plasma exchange, intravenous immunoglobulin, cyclosporine, or mycophenolate mofetil.

The combined administration includes co-administration, using separate formulations or a single pharmaceutical formulation, and consecutive administration in either order, wherein preferably there is a time period while both (or all) active agents simultaneously exert their biological activities.

Aside from administration of protein antagonists to the patient, the present application contemplates administration of antagonists by gene therapy. Such administration of nucleic acid encoding the antagonist is encompassed by the expression “administering an effective amount of an antagonist.” See, for example, WO 1996/07321 published Mar. 14, 1996 concerning the use of gene therapy to generate intracellular antibodies.

There are two major approaches to getting the nucleic acid (optionally contained in a vector) into the patient's cells: in vivo and ex vivo. For in vivo delivery the nucleic acid is injected directly into the patient, usually at the site where the antagonist is required. For ex vivo treatment, the patient's cells are removed, the nucleic acid is introduced into these isolated cells, and the modified cells are administered to the patient either directly or, for example, encapsulated within porous membranes that are implanted into the patient (see, e.g. U.S. Pat. Nos. 4,892,538 and 5,283,187). There are a variety of techniques available for introducing nucleic acids into viable cells. The techniques vary depending upon whether the nucleic acid is transferred into cultured cells in vitro, or in vivo in the cells of the intended host. Techniques suitable for the transfer of nucleic acid into mammalian cells in vitro include the use of liposomes, electroporation, microinjection, cell fusion, DEAE-dextran, the calcium phosphate precipitation method, etc. A commonly used vector for ex vivo delivery of the gene is a retrovirus.

The currently preferred in vivo nucleic acid transfer techniques include transfection with viral vectors (such as adenovirus, Herpes simplex I virus, or adeno-associated virus) and lipid-based systems (useful lipids for lipid-mediated transfer of the gene are DOTMA, DOPE and DC-Chol, for example). In some situations it is desirable to provide the nucleic acid source with an agent that targets the target cells, such as an antibody specific for a cell-surface membrane protein or the target cell, a ligand for a receptor on the target cell, etc. Where liposomes are employed, proteins that bind to a cell-surface membrane protein associated with endocytosis may be used for targeting and/or to facilitate uptake, e.g. capsid proteins or fragments thereof tropic for a particular cell type, antibodies for proteins that undergo internalization in cycling, and proteins that target intracellular localization and enhance intracellular half-life. The technique of receptor-mediated endocytosis is described, for example, by Wu et al. J. Biol. Chem. 262:4429-4432 (1987); and Wagner et al. Proc. Natl. Acad. Sci. USA 87:3410-3414 (1990). For review of the currently known gene marking and gene therapy protocols, see Anderson et al. Science 256:808-813 (1992). See also WO 1993/25673 and the references cited therein.

VI. Articles of Manufacture

In another embodiment of the invention, an article of manufacture containing materials useful for the treatment of the diseases or disorders described above is provided. The article of manufacture comprises a container and a label or package insert on or associated with the container. Suitable containers include, for example, bottles, vials, syringes, etc. The containers may be formed from a variety of materials such as glass or plastic. The container holds or contains a composition that is effective for treating the disease or disorder of choice and may have a sterile access port (for example, the container may be an intravenous solution bag or a vial having a stopper pierceable by a hypodermic injection needle). At least one active agent in the composition is the antagonist that binds a B-cell surface marker. The label or package insert indicates that the composition is used for treating a patient having or predisposed to an autoimmune disease, such as those listed herein. The article of manufacture may further comprise a second container comprising a pharmaceutically acceptable diluent buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

Further details of the invention are illustrated by the following non-limiting Examples. The disclosures of all citations in the specification are expressly incorporated herein by reference.

### EXAMPLE 1

### Humanization of 2H7 Anti-CD20 Murine Monoclonal Antibody

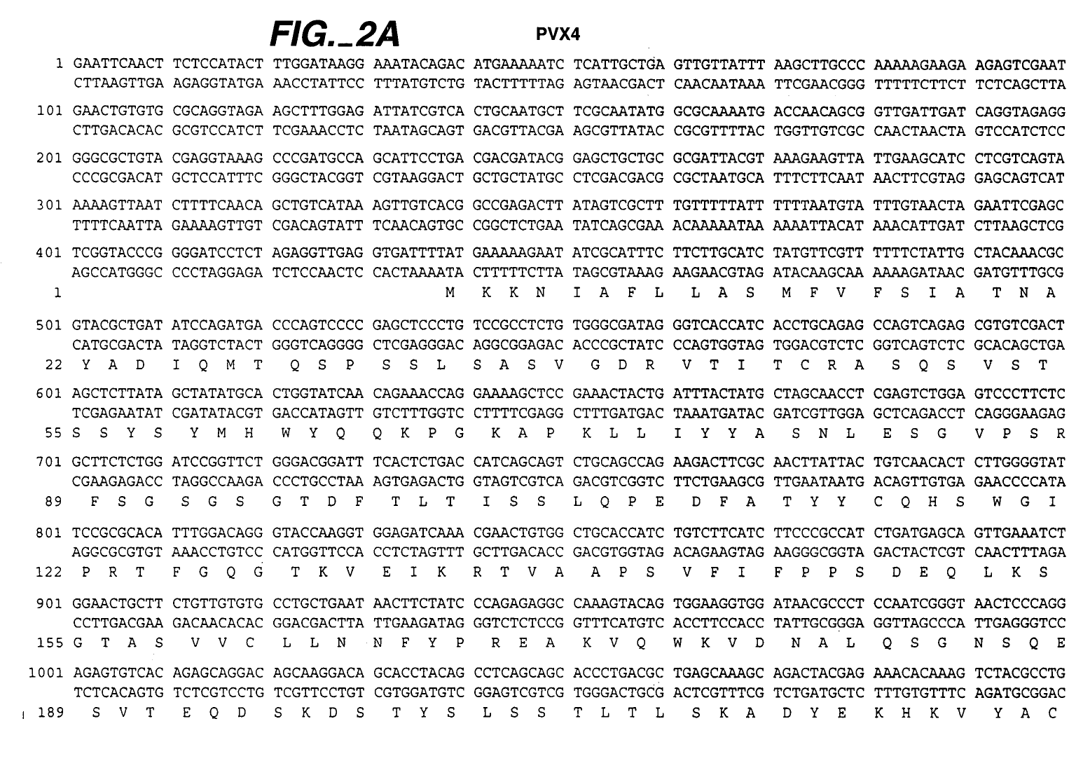

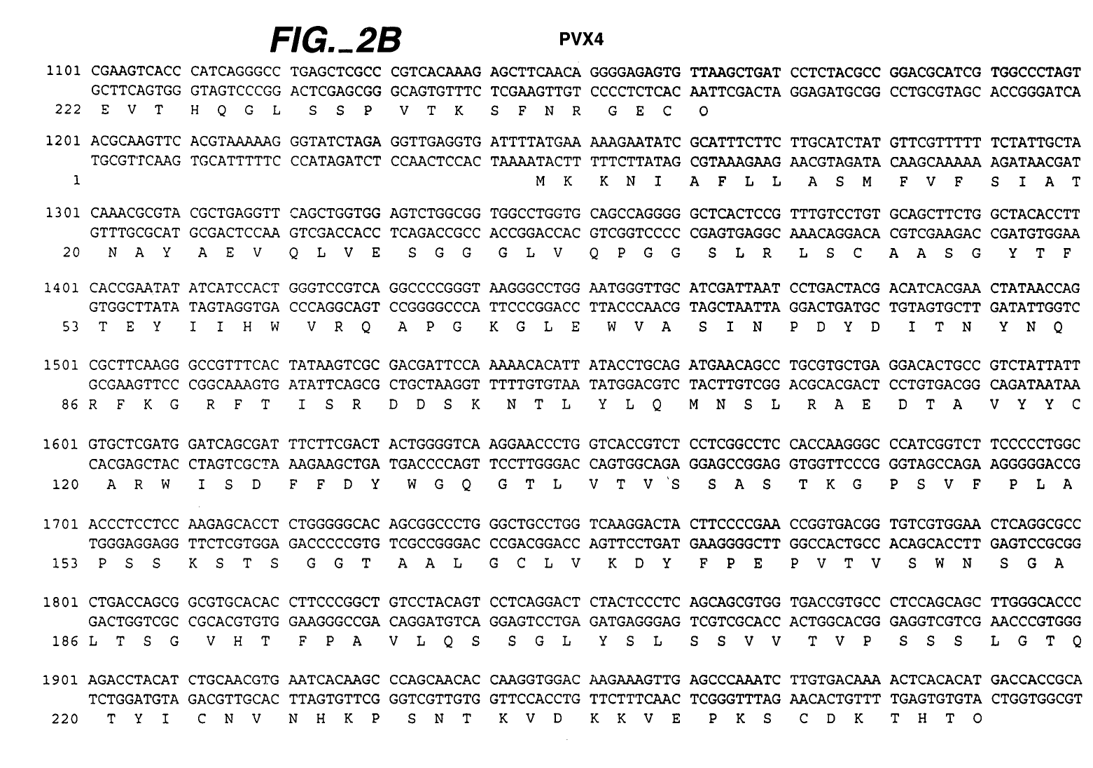

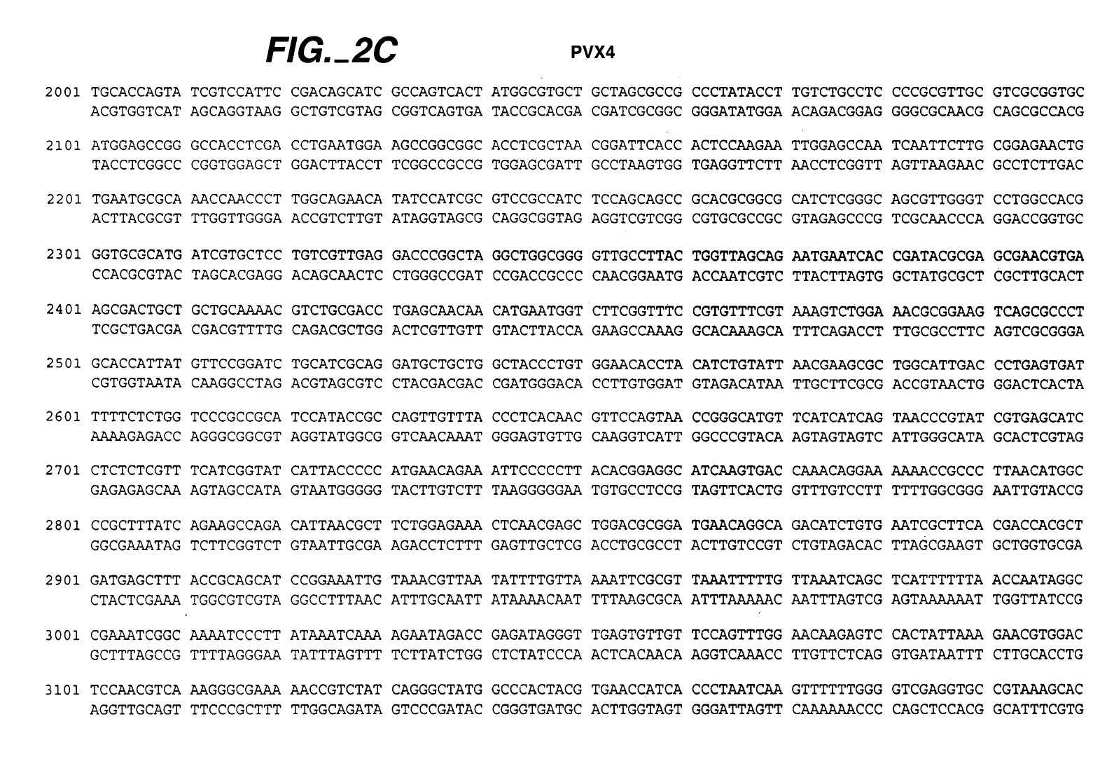

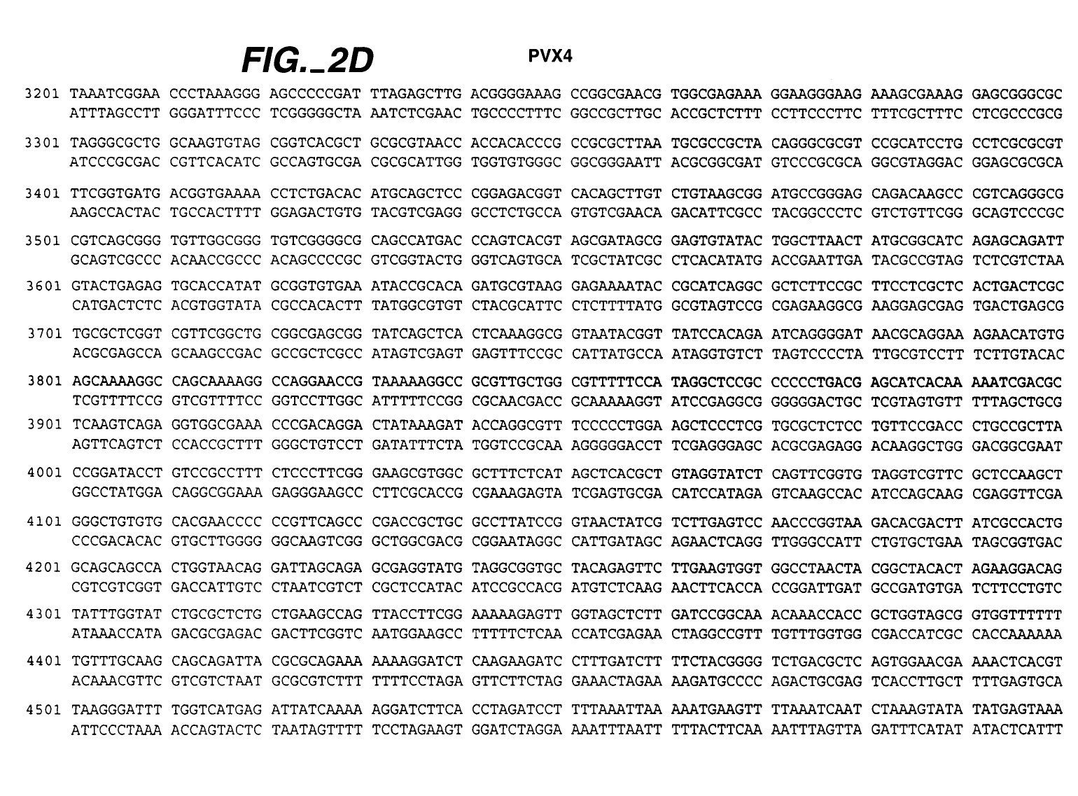

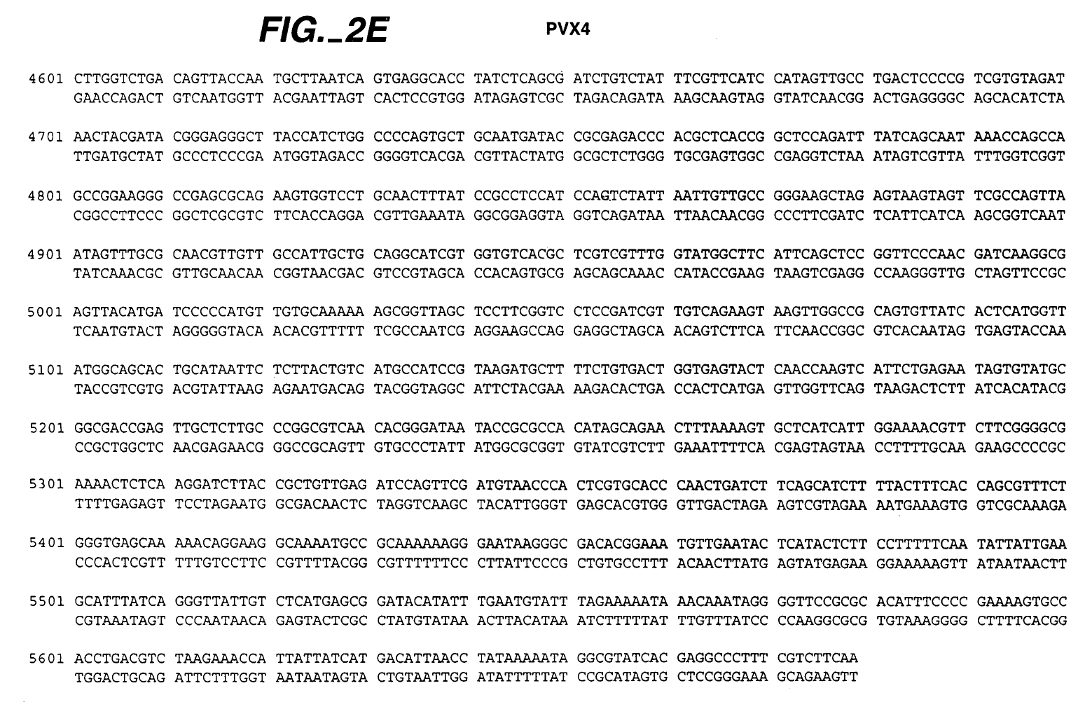

Humanization of the murine anti-human CD20 antibody, 2H7 (also referred to herein as m2H7, m for murine), was carried out in a series of site-directed mutagenesis steps. The murine 2H7 antibody variable region sequences and the chimeric 2H7 with the mouse V and human C have been described; see, e.g., U.S. Pat. Nos. 5,846,818 and 6,204,023. The CDR residues of 2H7 were identified by comparing the amino acid sequence of the murine 2H7 variable domains (disclosed in U.S. Pat. No. 5,846,818) with the sequences of known antibodies (Kabat et al. Sequences of proteins of immunological interest, Ed. 5. Public Health Service, National Institutes of Health, Bethesda, Md. (1991)). The CDR regions for the light and heavy chains were defined based on sequence hypervariability (Kabat et al., supra) and are shown in FIG. 1A and FIG. 1B, respectively. Using synthetic oligonucleotides (Table 2), site-directed mutagenesis (Kunkel, Proc. Natl. Acad. Sci. 82:488-492 (1985)) was used to introduce all six of the murine 2H7CDR regions into a complete human Fab framework corresponding to a consensus sequence V_{κ}I, V_{H}III (V_{L} kappa subgroup I, V_{H} subgroup III) contained on plasmid pVX4 (FIG. 2).

The phagemid pVX4 (FIG. 2) was used for mutagenesis as well as for expression of F(ab)s in E. coli. Based on the phagemid pb0720, a derivative of pB0475 (Cunningham et al. Science 243:1330-1336 (1989)), pVX4 contains a DNA fragment encoding a humanized consensus κ-subgroup I light-chain (V_{L}κI-C_{L}) and a humanized consensus subgroup III heavy-chain (V_{H}III-C_{H}1) anti-IFN-α (interferon-α) antibody. pVX4 also has an alkaline phosphatase promoter and Shine-Dalgarno sequence both derived from another previously described pUC119-based plasmid, pAK2 (Carter et al. Proc. Natl. Acad. Sci. USA 89: 4285 (1992)). A unique Spel restriction site was introduced between the DNA encoding the F(ab) light and heavy chains. The first 23 amino acids in both anti-IFN-α heavy and light chains are the STII secretion signal sequence (Chang et al. Gene 55:189-196 (1987)).

To construct the CDR-swap version of 2H7 (2H7.v2), site-directed mutagenesis was performed on a deoxyuridine-containing template of pVX4; all six CDRs of anti-IFN-α were changed to the murine 2H₇CDRs. The resulting-molecule is referred to as humanized 2H7 version 2 (2H7.v2), or the “CDR-swap version” of 2H7; it has the m2H₇CDR residues with the consensus human FR residues shown in FIGS. 1A and 1B. Humanized 2H7.v2 was used for further humanization.

Table 2 shows the oligonucleotide sequence used to create each of the murine 2H7 (m2H7) CDRs in the H and L chain. For example, the CDR-H1 oligonucleotide was used to recreate the m2H7H chain CDR1. CDR-H1, CDR-H2 and CDR-H3 refer to the H-chain CDR1, CDR2 and CDR3, respectively; similarly, CDR-L1, CDR-L2 and CDR-L3 refer to each of the L-chain CDRs. The substitutions in CDR-H2 were done in two steps with two oligonucleotides, CDR-H2A and CDR-H2B.

TABLE 2

Oligonucleotide sequences used for construction
of the CDR-swap of murine 2H7 CDRs into a human
framework in pVX4. Residues changed by each
oligonucleotide are underlined.

| | |
|---|---|
| Substitution | Oligonucleotide sequence |

| | |
|---|---|
| CDR-H1 | C TAC ACC TTC ACG AGC TAT AAC ATG CAC TGG |
| | GTC CG |
| | (SEQ ID NO:31) |
| CDR-H2A | G ATT AAT CCT GAC AAC GGC GAC ACG AGC TAT |
| | AAC CAG AAG TTG AAG GGG CG |
| | (SEQ ID NO:32) |
| CDR-H2B | GAA TGG GTT GCA GCG ATC TAT CCT GGC AAC |
| | GCG GAC AC |
| | (SEQ ID NO:33) |
| CDR-H3 | AT TAT TGT GCT CGA GTG GTC TAC TAT AGC AAC |
| | AGC TAC TGG TAC TTC GAC GTC TGG GGT CAA |
| | GGA |
| | (SEQ ID NO:34) |
| CDR-L1 | C TGC ACA GCC AGC TCT TCT GTC AGC TAT ATG |
| | CAT TG |
| | (SEQ ID NO:35) |
| CDR-L2 | AA CTA CTG ATT TAC GCT CCA TCG AAC CTC GCG |
| | TCT GGA GTC C |
| | (SEQ ID NO:36) |
| CDR-L3 | TAT TAC TGT CAA CAG TGG AGC TTC AAT CCG |
| | CCC ACA TTT GGA CAG |
| | (SEQ ID NO:37) |

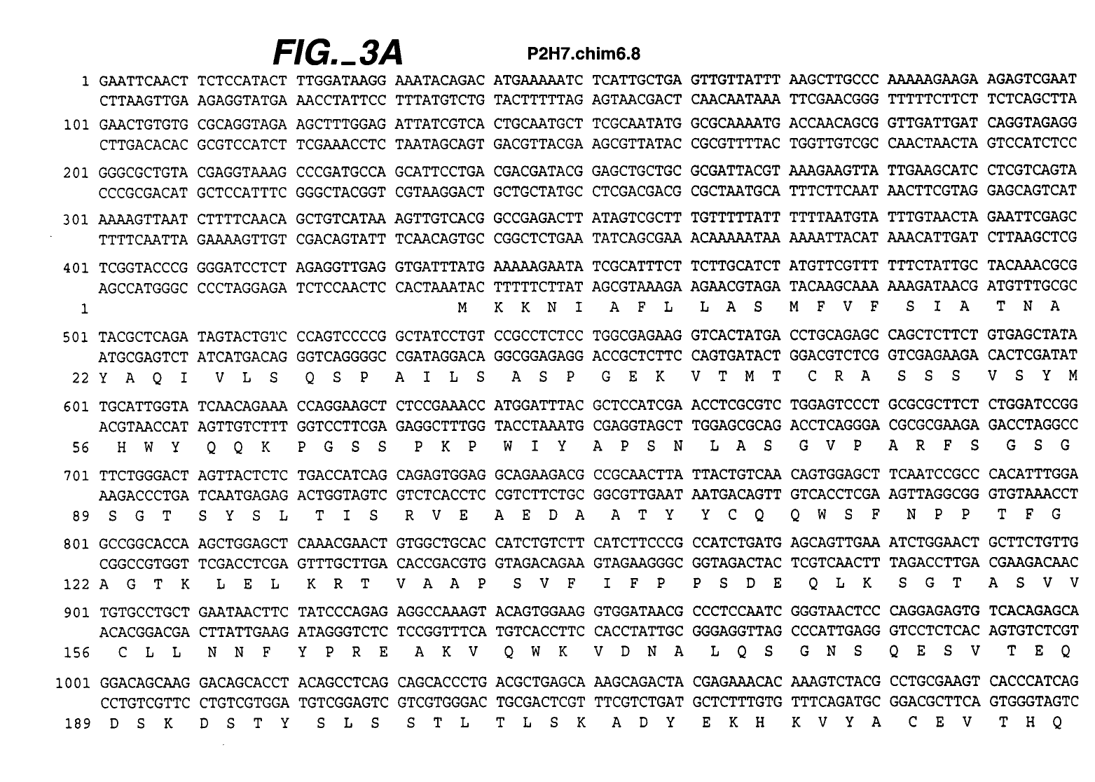

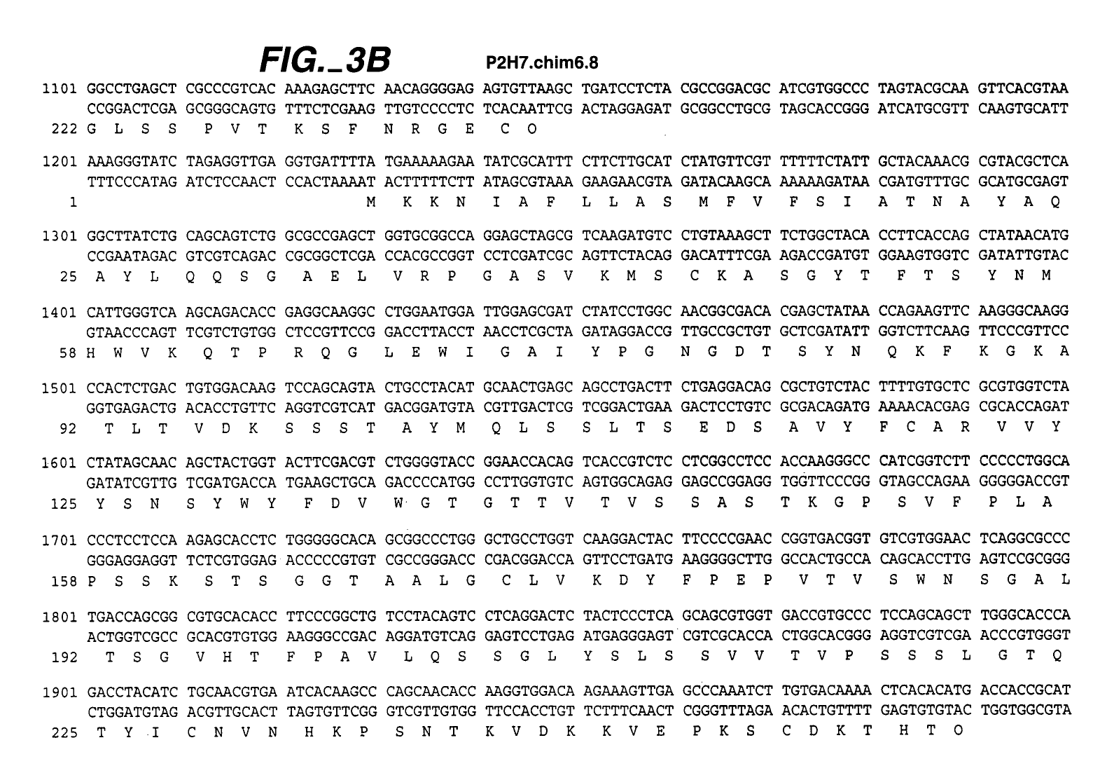

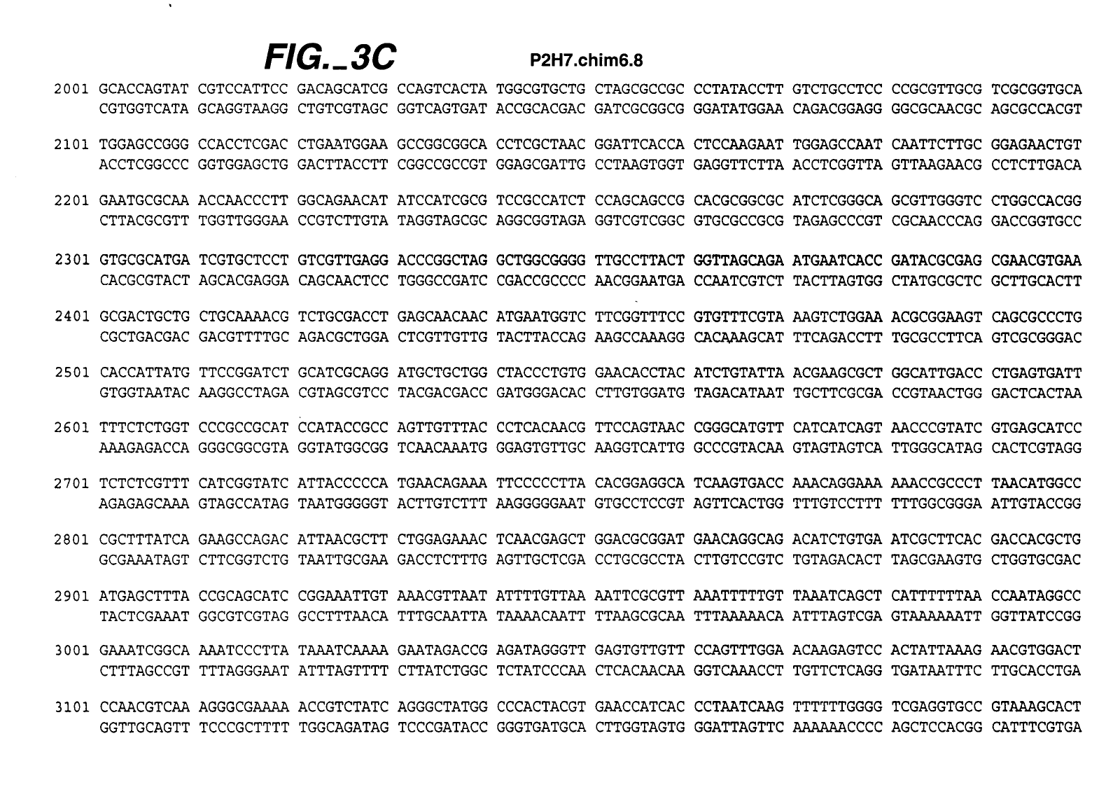

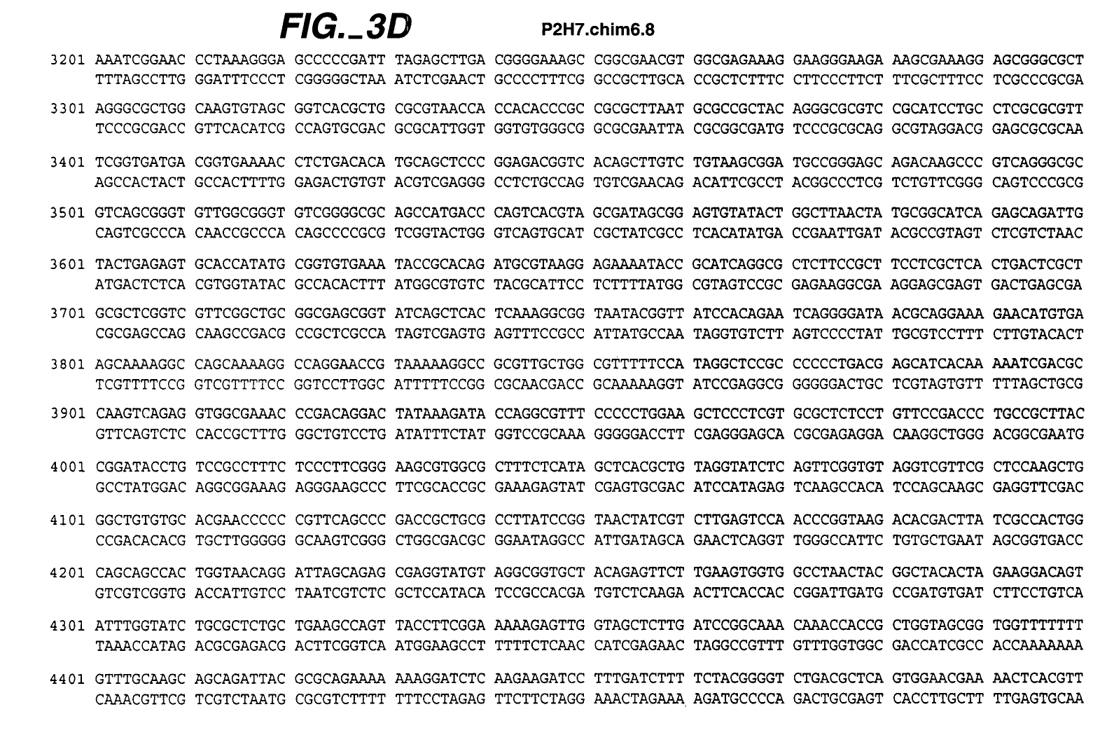

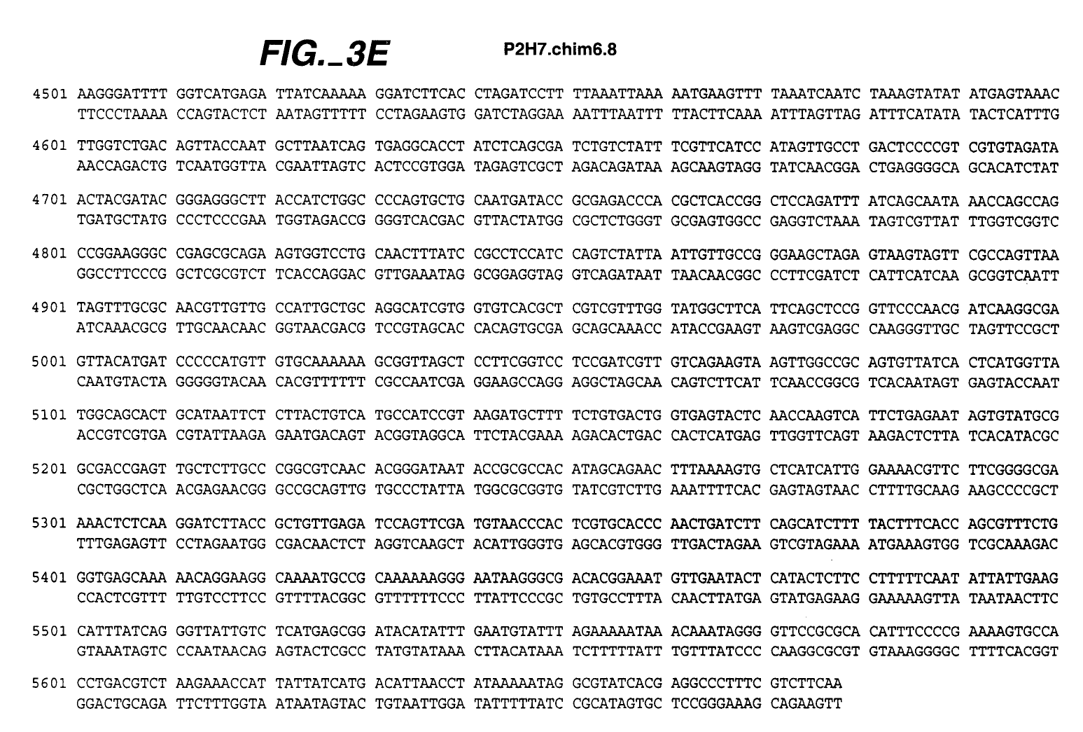

For comparison with humanized constructs, a plasmid expressing a chimeric 2H₇Fab (containing murine V_{L} and V_{H} domains, and human C_{L} and CH₁ domains) was constructed by site-directed mutagenesis (Kunkel, supra) using synthetic oligonucleotides to introduce the murine framework residues into 2H7.v2. The sequence of the resulting plasmid construct for expression of the chimeric Fab known as 2H7.v6.8, is shown in FIG. 3. Each encoded chain of the Fab has a 23-amino-acid STII secretion signal sequence as described for pVX4 (FIG. 2) above.

Based on a sequence comparison of the murine 2H7 framework residues with the human V_{κ}I, V_{H}III consensus framework (FIGS. 1A and 1B) and previously humanized antibodies (Carter et al. Proc. Natl. Acad. Sci. USA 89:4285-4289 (1992)), several framework mutations were introduced into the 2H7.v2 Fab construct by site-directed mutagenesis. These mutations result in a change of certain human consensus framework residues to those found in the murine 2H7 framework, at sites that might affect CDR conformations or antigen contacts. Version 3 contained V_{H}(R71V, N73K), version 4 contained V_{H}(R71V), version 5 contained V_{H}(R71V, N73K) and V_{L}(L46P), and version 6 contained V_{H}(R7 IV, N73K) and V_{L}(LM6P, L47W).

Humanized and chimeric Fab versions of m2H7 antibody were expressed in E. coli and purified as follows. Plasmids were transformed into E. coli strain XL-1 Blue (Stratagene, San Diego, Calif.) for preparation of double-and single-stranded DNA. For each variant, both light and heavy chains were completely sequenced using the dideoxynucleotide method (SEQUENASE® labeled primer cycle sequencing, U.S. Biochemical Corp.). Plasmids were transformed into E. coli strain 16C9, a derivative of MM294, plated onto LB plates containing 5 μg/ml carbenicillin, and a single colony was selected for protein expression. The single colony was grown in 5 ml LB-100 μg/ml carbenicillin for 5-8 h at 37° C. The 5 ml culture was added to 500 ml AP5-100 μg/ml carbenicillin and allowed to grow for 16 h in a 4-L baffled shake flask at 37° C. AP5 media consists of: 1.5 g glucose, 11.0 g HYCASE SF™ (casein hydrolysate), 0.6 g yeast extract (certified), 0.19 g anhydrous MgSO₄, 1.07 g NH₄Cl, 3.73 g KCl, 1.2 g NaCl, 120 ml 1 M triethanolamine, pH 7.4, to 1 L water and then sterile filtered through a 0.1 μm SEAKLEEN® biocide filter.

Cells were harvested by centrifugation in a 1-L centrifuge bottle (Nalgene) at 3000×g and the supernatant was removed. After freezing for 1 h, the pellet was resuspended in 25 ml cold 10 mM MES-10 mM EDTA, pH 5.0 (buffer A). 250 μl of 0.1M phenylmethylsulphonyl fluoride (PMSF) (Sigma) was added to inhibit proteolysis and 3.5 ml of stock 10 mg/ml hen egg white lysozyme (Sigma) was added to aid lysis of the bacterial cell wall. After gentle shaking on ice for 1 h, the sample was centrifuged at 40,000×g for 15 min. The supernatant was brought to 50 ml with buffer A and loaded onto a 2-ml DEAE column equilibrated with buffer A. The flow-through was then applied to a protein G-SEPHAROSE CL-4B™ agarose (Pharmacia) chromatography column (0.5-ml bed volume) equilibrated with buffer A. The column was washed with 10 ml buffer A and eluted with 3 ml of 0.3 M glycine, pH 3.0, into 1.25 ml of 1 M TRIS, pH 8.0. The F(ab) was then buffer exchanged into phosphate-buffered saline (PBS) using a CENTRICON-30® centrifugal filter device (Amicon) and concentrated to a final volume of 0.5 ml. SDS-PAGE gels of all F(ab)s were run to ascertain purity, and the molecular weight of each variant was verified by electrospray mass spectrometry.

In cell-based ELISA binding assays (described below), the binding of Fabs, including chimeric 2H₇Fab, to CD20 was difficult to detect. Therefore, the 2H₇Fab versions were reformatted as full-length IgG1 antibodies for assays and further mutagenesis.

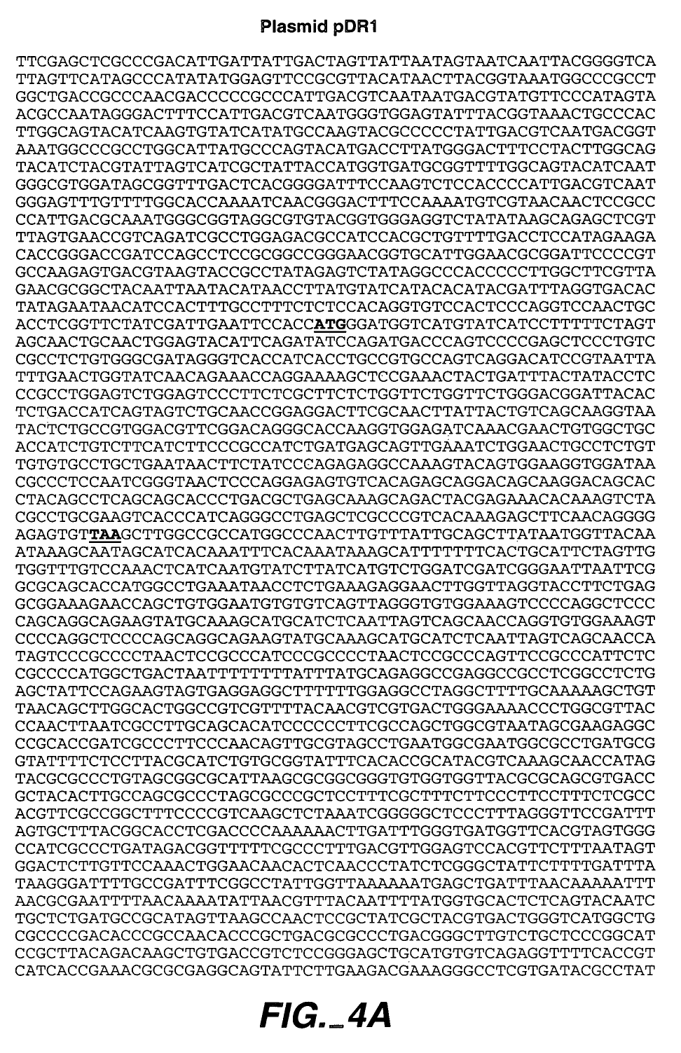

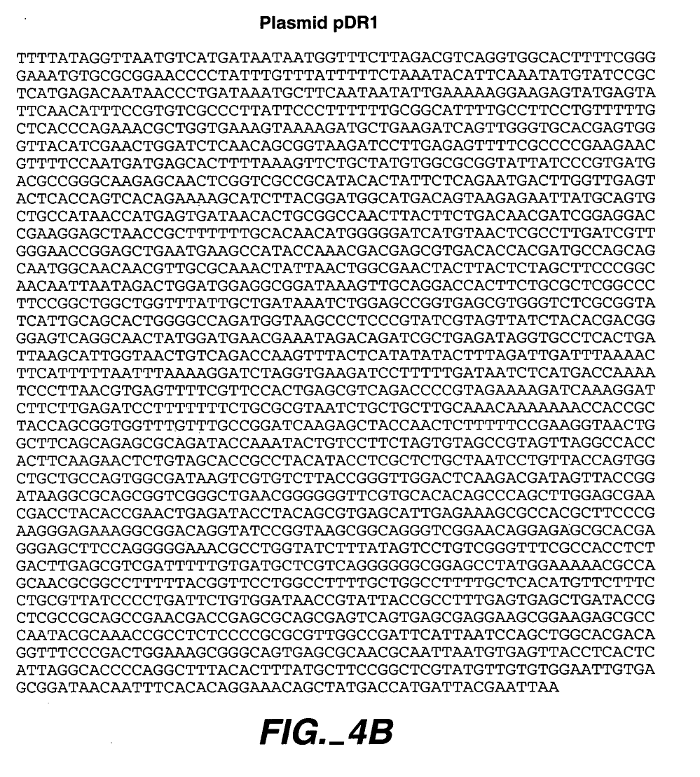

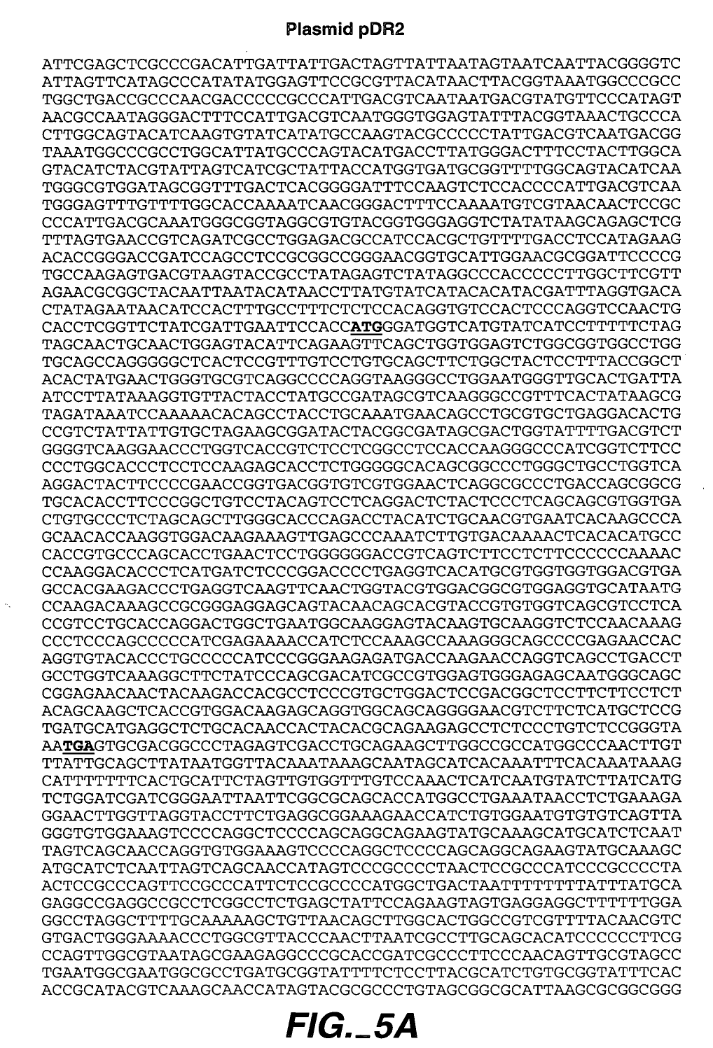

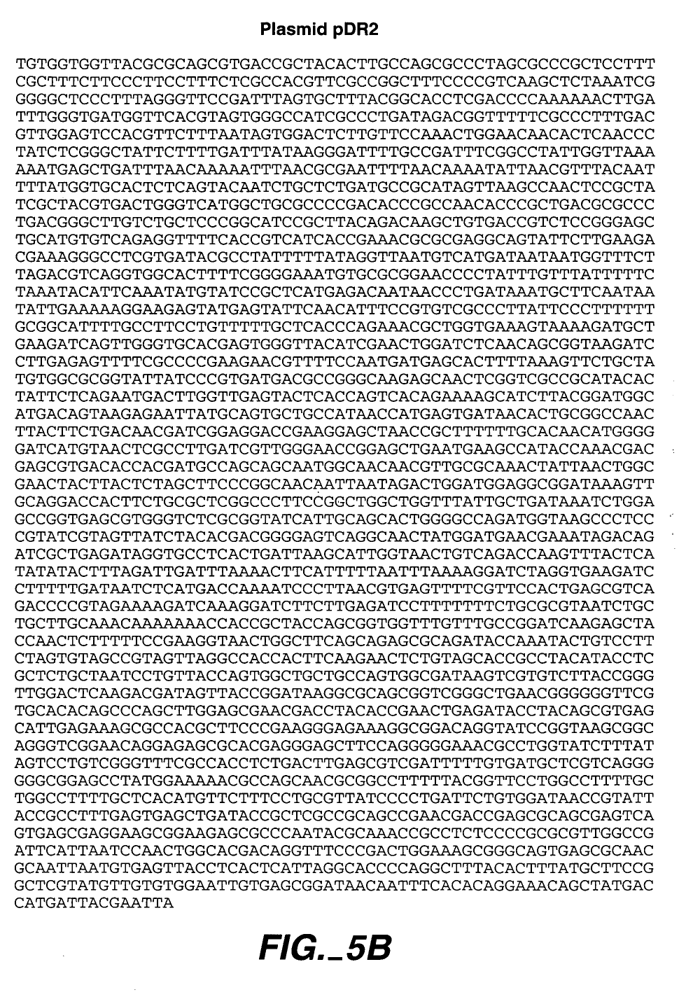

Plasmids for expression of full-length IgG's were constructed by subcloning the V_{L} and V_{H} domains of chimeric 2H7 (v6.8) Fab as well as humanized Fab versions 2 to 6 into previously described pRK vectors for mammalian cell expression (Gorman et al. DNA Prot. Eng. Tech. 2:3-10 (1990)). Briefly, each Fab construct was digested with EcoRV and BlpI to excise a V_{L} fragment, which was cloned into the EcoRV/BlpI sites of plasmid pDR1 (FIG. 4) for expression of the complete light chain (V_{L}-C_{L} domains). Additionally, each Fab construct was digested with PvuII and ApaI to excise a V_{H} fragment, which was cloned into the PvuII/ApaI sites of plasmid pDR2 (FIG. 5) for expression of the complete heavy chain (V_{H}-CH₁-hinge-CH₂-CH₃ domains). For each IgG variant, transient transfections were performed by cotransfecting a light-chain expressing plasmid and a heavy-chain expressing plasmid into an adenovirus-transformed human embryonic kidney cell line, 293 (Graham et al. J. Gen. Virol. 36:59-74 (1977)). Briefly, 293 cells were split on the day prior to transfection, and plated in serum-containing medium. On the following day, double-stranded DNA prepared as a calcium phosphate precipitate was added, followed by PADVANTAGE™ DNA (Promega, Madison, Wis.), and cells were incubated overnight at 37° C. Cells were cultured in serum-free medium and harvested after 4 days. Antibodies were purified from culture supernatants using protein A-SEPHAROSE CL-4B™ agarose chromatography, then buffer exchanged into 10 mM sodium succinate, 140 mM NaCl, pH 6.0, and concentrated using a CENTRICON-10® centrifugal filter device (Amicon). Protein concentrations were determined by quantitative amino acid analysis.

To measure relative binding affinities to the CD20 antigen, a cell-based ELISA assay was developed. Human B-lymphoblastoid WIL2-S cells (ATCC CRL 8885, American Type Culture Collection, Manassas, Va.) were grown in RPMI 1640 supplemented with 2 mM L-glutamine, 20 mM HEPES, pH 7.2 and 10% heat-inactivated fetal bovine serum in a humidified 5% CO₂ incubator. The cells were washed with PBS containing 1% fetal bovine serum (FBS) (assay buffer) and seeded at 250-300,000 cell/well in 96-well round bottom plates (Nunc, Roskilde, Denmark). Two-fold serially diluted standard (15.6-1000 ng/ml of 2H7 v6.8 chimeric IgG) and threefold serially diluted samples (2.7-2000 ng/ml) in assay buffer were added to the plates. The plates were buried in ice and incubated for 45 min. To remove the unbound antibody, 0.1 mL assay buffer was added to the wells. Plates were centrifuged and supernatants were removed. Cells were washed two more times with 0.2 mL assay buffer. Antibody bound to the plates was detected by adding peroxidase-conjugated goat anti-human Fc antibody (Jackson ImmunoResearch, West Grove, Pa.) to the plates. After a 45-min incubation, cells were washed as described before. TMB substrate (3,3′,5,5′-tetramethyl benzidine; Kirkegaard & Perry Laboratories, Gaithersburg, Md.) was added to the plates. The reaction was stopped by adding 1 M phosphoric acid. Titration curves were fit with a four-parameter nonlinear regression curve-fitting program (KALEIDAGRAPH™, Synergy software, Reading, Pa.). The absorbance at the midpoint of the titration curve (mid-OD) and its corresponding concentration of the standard were determined. Then the concentration of each variant at this mid-OD was determined, and the concentration of the standard was divided by that of each variant. Hence, the values are a ratio of the binding of each variant relative to the standard. Standard deviations in relative affinity (equivalent concentration) were generally +/−10% between experiments.

As shown in Table 3, binding of the CDR-swap variant (v.2) was extremely reduced compared to chimeric 2H7 (v.6.8). However, versions 3 to 6 showed improved binding. To determine the minimum number of mutations that might be required to restore binding affinity to that of chimeric 2H7, additional mutations and combinations of mutations were constructed by site-direct mutagenesis to produce variants 7 to 17 as indicated in Table 4. In particular, these included V_{H} mutations A49G, F67A, 169L, N73K, and L78A; and V_{L} mutations M4L, M331, and F71Y. Versions 16 and 17 showed the best relative binding affinities, within 2-fold of that of the chimeric version, with no significant difference (s.d. =+/−10%) between the two. To minimize the number of mutations, version 16, having only 4 mutations of human framework residues to murine framework residues (Table 4), was therefore chosen as the humanized form for additional characterization.

TABLE 3
Relative binding affinity of humanized 2H7 IgG variants to CD20
compared to chimeric 2H7 using cell-based ELISA. The relative
binding is expressed as the concentration of the chimeric 2H7 over the
concentration of the variant required for equivalent binding; hence a ratio
<1 indicates weaker affinity for the variant. Standard deviation in
relative affinity determination averaged +/−10%. Framework
substitutions in the variable domains are relative to the CDR-swap
version according to the numbering system of Kabat (Kabat et al., supra).

| 2H7 | Heavy-Chain (V_{H}) | Light-Chain (V_{L}) | Relative |
|---|---|---|---|
| version | substitutions | substitutions | binding |
| 6.8 | (Chimera) | (Chimera) | -1- |
| 2 | (CDR swap) | (CDR swap) | 0.01 |
| 3 | R71V, N73K | (CDR swap) | 0.21 |
| 4 | R71V | (CDR swap) | 0.21 |
| 5 | R71V, N73K | L46P | 0.50 |
| 6 | R71V, N73K | L46P, L47W | 0.58 |
| 7 | R71V | L46P | 0.33 |
| 8 | R71V, L78A | L46P | 0.19 |
| 9 | R71V, F67A | L46P | 0.07 |
| 10 | R71V, F67A, I69L | L46P | 0.12 |
| 11 | R71V, F67A, L78A | L46P | 0.19 |
| 12 | R71V | L46P, M4L | 0.32 |
| 13 | R71V | L46P, M33I | 0.31 |
| 14 | R71V | L46P, F71Y | 0.25 |
| 15 | R71V | L46P, M4L, M33I | 0.26 |
| 16 | R71V, N73K, A49G | L46P | 0.65 |
| 17 | R71V, N73K, A49G | L46P, L47W | 0.67 |

TABLE 4

Oligonucleotide sequences used for construction
of mutations VH(A49G, R71V, N73K) and VL(L46P)
in humanized 2H7 version 16 (2H7.v16).
Underlined codons encode the indicated amino
acid substitutions. For V_{H} (R71V, N73K) and
V_{L} (L46P), the oligonucleotides are shown as
the sense strand since these were used for
mutagenesis on the Fab template, while for
V_{H} (A49G), the oligonucleotide is shown as the
anti-sense strand, since this was used with the
pRK (IgG heavy-chain) template. The protein
sequence of version 16 is shown in FIG. 6 and
FIG. 7.

| | |
|---|---|
| Substitution | Oligonucleotide sequence |

| | |
|---|---|
| V_{H} (R71V, N73K) | GT TTC ACT ATA AGT GTC GAC AAG TCC |
| | AAA AAC ACA TT |
| | (SEQ ID NO:38) |
| V_{H} (A49G) | GCCAGGATAGATGGCGCCAACCCATTCCAGGCC |
| | (SEQ ID NO:39) |
| V_{L} (L46P) | AAGCTCCGAAACCACTGATTTACGCT |
| | (SEQ ID NO:40) |

### EXAMPLE 2

### Antigen-Binding Determinants (Paratopes) of 2H7

Alanine substitutions (Cunningham & Wells, Science 244:1081-1085 (1989)) were made in 2H7.v 16 or 2H7.v17 in order to test the contributions of individual side chains of the antibody in binding to CD20. IgG variants were expressed in 293 cells from pDR1 and pDR2 vectors, purified, and assayed for relative binding affinity as described above. Several alanine substitutions resulted in significant decreases in relative binding to CD20 on WIL-2S cells (Table 5).

TABLE 5
Effects of alanine substitutions in the CDR regions of humanized 2H7.v16
measured using cell-based ELISA (WIL2-S cells). The relative binding is
expressed as the concentration of the 2H7.v16 parent over the
concentration of the variant required for equivalent binding; hence a ratio
<1 indicates weaker affinity for the variant; a ratio >1 indicates higher
affinity for the variant. Standard deviation in relative affinity
determination averaged +/−10%. Framework substitutions in the
variable domains are relative to 2H7.v16 according to the numbering
system of Kabat (Kabat et al., supra). NBD means no detectable binding.
The two numbers for version 45 are from separate experiments.

| 2H7 | CDR | Heavy-chain | Light-chain | Relative |
|---|---|---|---|---|
| version | location | substitutions | substitutions | binding |
| 16 | — | — | — | -1- |
| 140 | H1 | G26A | — | 0.63 |
| 141 | H1 | Y27A | — | 0.47 |
| 34 | H1 | T28A | — | 0.86 |
| 35 | H1 | F29A | — | 0.07 |
| 36 | H1 | T30A | — | 0.81 |
| 37 | H1 | S31A | — | 0.97 |
| 142 | H1 | Y32A | — | 0.63 |
| 143 | H1 | N33A | — | NDB |
| 144 | H1 | M34A | — | 1.2 |
| 145 | H1 | H35A | — | <0.25 |
| 146 | H2 | A50G | — | 0.31 |
| 147 | H2 | I51A | — | 0.65 |
| 38 | H2 | Y52A | — | 0.01 |
| 148 | H2 | P52aA | — | 0.66 |
| 39 | H2 | G53A | — | 0.89 |
| 67 | H2 | N54A | — | 1.4 |
| 40 | H2 | G55A | — | 0.79 |
| 41 | H2 | D56A | — | 2.0 |
| 89 | H2 | T57A | — | 0.61 |
| 90 | H2 | S58A | — | 0.92 |
| 91 | H2 | Y59A | — | 0.74 |
| 92 | H2 | N60A | — | 0.80 |
| 93 | H2 | Q61A | — | 0.83 |
| 94 | H2 | K62A | — | 0.44 |
| 95 | H2 | F63A | — | 0.51 |
| 83 | H2 | V71A | — | 0.96 |
| 149 | H2 | K64A | — | 0.82 |
| 150 | H2 | G65A | — | 1.2 |
| 153 | H3 | V95A | — | 0.89 |
| 42 | H3 | V96A | — | 0.98 |
| 43 | H3 | Y97A | — | 0.63 |
| 44 | H3 | Y98A | — | 0.40 |
| 45 | H3 | S99A | — | 0.84; 0.92 |
| 46 | H3 | N100A | — | 0.81 |
| 47 | H3 | S100aA | — | 0.85 |
| 48 | H3 | Y100bA | — | 0.78 |
| 49 | H3 | W100cA | — | 0.02 |
| 59 | H3 | Y100dA | — | 0.98 |
| 60 | H3 | F100eA | — | NDB |
| 61 | H3 | D101A | — | 0.31 |
| 151 | H3 | V102A | — | 1.1 |
| 117 | L1 | — | R24A | 0.85 |
| 118 | L1 | — | A25G | 0.86 |
| 119 | L1 | — | S26A | 0.98 |
| 120 | L1 | — | S27A | 0.98 |
| 121 | L1 | — | S28A | 1.0 |
| 122 | L1 | — | V29A | 0.41 |
| 50 | L1 | — | S30A | 0.96 |
| 51 | L1 | — | Y32A | 1.0 |
| 123 | L1 | — | M33A | 1.0 |
| 124 | L1 | — | H34A | 0.21 |
| 125 | L2 | — | A50G | 0.92 |
| 126 | L2 | — | P51A | 0.88 |
| 52 | L2 | — | S52A | 0.80 |
| 53 | L2 | — | N53A | 0.76 |
| 54 | L2 | — | L54A | 0.60 |
| 127 | L2 | — | A55G | 1.1 |
| 128 | L2 | — | S56A | 1.1 |
| 129 | L3 | — | Q89A | 0.46 |
| 130 | L3 | — | Q90A | <0.22 |
| 55 | L2 | — | W91A | 0.88 |
| 56 | L3 | — | S92A | 1.1 |
| 57 | L3 | — | F93A | 0.36 |
| 58 | L3 | — | N94A | 0.61 |
| 131 | L3 | — | P95A | NDB |
| 132 | L3 | — | P96A | 0.18 |
| 133 | L3 | — | T97A | <0.22 |

### EXAMPLE 3

### Additional Mutations within 2H7CDR Regions

Substitutions of additional residues and combinations of substitutions at CDR positions that were identified as important by Ala-scanning were also tested. Several combination variants, particularly v.96, appeared to bind more tightly than v.16.

TABLE 6
Effects of combinations of mutations and non-alanine substitutions in
the CDR regions of humanized 2H7.v16 measured using cell-based
ELISA (WIL2-S cells). The relative binding to CD20 is expressed as the
concentration of the 2H7.v16 parent over the concentration of the variant
required for equivalent binding; hence, a ratio <1 indicates weaker
affinity for the variant; a ratio >1 indicates higher affinity for the variant.
Standard deviation in relative affinity determination averaged +/−10%.
Framework substitutions in the variable domains are relative to 2H7.v16
according to the numbering system of Kabat (Kabat et al., supra).

| 2H7 | Heavy-chain | Light-chain | Relative |
|---|---|---|---|
| version | substitutions | substitutions | binding |
| 16 | — | — | -1- |
| 96 | D56A, N100A | S92A | 3.5 |
| 97 | S99T, N100G, Y100bI | — | 0.99 |
| 98 | S99G, N100S, Y100bI | — | 1.6 |
| 99 | N100G, Y100bI | — | 0.80 |
| 101 | N54S, D56A | — | 1.7 |
| 102 | N54K, D56A | — | 0.48 |
| 103 | D56A, N100A | — | 2.1 |
| 104 | S99T, N100G | — | 0.81 |
| 105 | S99G, N100S | — | 1.1 |
| 106 | N100G | — | ˜1 |
| 167 | S100aG, Y100bS | — | |
| 136 | D56A, N100A | S56A, S92A | 2.6 |
| 137 | D56A, N100A | A55G, S92A | 2.1 |
| 156 | D56A, N100A | S26A, S56A, S92A | 2.1 |
| 107 | D56A, N100A, Y100bI | S92A | not expressed |
| 182 | Y27W | — | |
| 183 | Y27F | — | |
| 184 | F29Y | — | |
| 185 | F29W | — | |
| 186 | Y32F | — | |
| 187 | Y32W | — | |
| 188 | N33Q | — | |
| 189 | N33D | — | |
| 190 | N33Y | — | |
| 191 | N33S | — | |
| 208 | H35S | — | |
| 209 | A50S | — | |
| 210 | A50R | — | |
| 211 | A50V | — | |
| 212 | A50L | — | |
| 168 | Y52W | — | |
| 169 | Y52F | — | 0.75 |
| 170 | N54D | — | 0.25 |
| 171 | N54S | — | 1.2 |
| 172 | D56K | — | 1 |
| 173 | D56R | — | |
| 174 | D56H | — | 1.5 |
| 175 | D56E | — | 1.2 |
| 213 | D56S | — | |
| 214 | D56G | — | |
| 215 | D56N | — | |
| 216 | D56Y | — | |
| 176 | Y59W | — | |
| 177 | Y59F | — | |
| 180 | K62R | — | |
| 181 | K62D | — | |
| 178 | F63W | — | |
| 179 | F63Y | — | |
| 157 | Y97W | — | 0.64 |
| 158 | Y97F | — | 1.2 |
| 159 | Y98W | — | 0.64 |
| 160 | Y98F | — | 0.88 |
| 106 | N100G | — | |
| 161 | W100cY | — | 0.05 |
| 162 | W100cF | — | 0.27 |
| 163 | F100eY | — | 0.59 |
| 164 | F100eW | — | 0.71 |
| 165 | D101N | — | 0.64 |
| 166 | S99G, N100G, S100aD, | — | 0.99 |
| | Y100b deleted | | |
| 217 | V102Y | — | 1.0 |
| 207 | — | H34Y | |
| 192 | — | Q89E | |
| 193 | — | Q89N | |
| 194 | — | Q90E | |
| 195 | — | Q90N | |
| 196 | — | W91Y | |
| 197 | — | W91F | |
| 205 | — | S92N | |
| 206 | — | S92G | |
| 198 | — | F93Y | |
| 199 | — | F93W | |
| 204 | — | F93S, N94Y | |
| 200 | — | P96L | |
| 201 | — | P96Y | |
| 202 | — | P96W | |
| 203 | — | P96R | |

### EXAMPLE 4

### Mutations at Sites of Framework Humanization Substitutions

Substitutions of additional residues at framework positions that were changed during humanization were also tested in the 2H7.v16 background. In particular, alternative framework substitutions that were neither found in the murine 2H7 parent nor the human consensus framework were made at V_{L}(P46) and V_{H}(G49, V71, and K73).

These substitutions generally led to little change in relative binding (Table 7), indicating that there is some flexibility in framework residues at these positions.

TABLE 7
Relative binding in a cell-based (WIL2-S) assay of framework
substitutions. IgG variants are shown with mutations with respect to the
2H7.v16 background. The relative binding is expressed as the
concentration of the 2H7.v6.8 chimera over the concentration of the
variant required for equivalent binding; hence, a ratio <1 indicates
weaker affinity for the variant; a ratio >1 indicates higher affinity for
the variant. Standard deviation in relative affinity determination averaged
+/−10%. Framework substitutions in the variable domains are relative to
2H7.v16 according to the numbering system of Kabat (Kabat et al., supra).

| 2H7 | Heavy-chain | Light-chain | Relative |
|---|---|---|---|
| version | substitutions | substitutions | binding |
| 6.8 | (chimera) | (chimera) | -1- |
| 16 | — | — | 0.64 |
| 78 | K73R | — | 0.72 |
| 79 | K73H | — | 0.49 |
| 80 | K73Q | — | 0.58 |
| 81 | V71I | — | 0.42 |
| 82 | V71T | — | 0.58 |
| 83 | V71A | — | |
| 84 | G49S | — | 0.32 |
| 85 | G49L | — | |
| 86 | — | P46E | 0.22 |
| 87 | — | P46V | 0.51 |
| 88 | — | P46T | |
| 108 | G49A, V71T, K73R | S92A, M32L, P46T | 0.026* |
| 109 | G49A, A49G, V71T, K73R | S92A, M32L, P46T | 0.026* |
| 110 | K73R, D56A, N100A | S92A, M32L | Not |
| | | | expressed |
| 111 | G49A, V71T, K73R | — | 0.46* |
| 112 | G49A, A50G, V71T, K73R | — | 0.12* |

*Variants that were assayed with 2H7.v16 as the standard comparator; relative values are normalized to that of the chimera.

### EXAMPLE 5

### Humanized 2H7 Variants with Enhanced Effector Functions

Because 2H7 can mediate lysis of B cells through both CDC and ADCC, variants of humanized 2H7.v16 are sought with improved CDC and ADCC activity. Mutations of certain residues within the Fc regions of other antibodies have been described (Idusogie et al. J. Immunol. 166:2571-2575 (2001)) for improving CDC through enhanced binding to the complement component C1q. Mutations have also been described (Shields et al. J. Biol. Chem. 276:6591-6604 (2001); Presta et al. Biochem. Soc. Trans. 30:487-490 (2002)) for improving ADCC through enhanced IgG binding to activating Fcγ receptors and reduced IgG binding to inhibitory Fcγ receptors. In particular, three mutations have been identified for improving CDC and ADCC activity: S298A/E333A/K334A (also referred to herein as a triple-Ala mutant or variant; numbering in the Fc region is according to the EU numbering system; Kabat et al., supra), as described (Idusogie et al., supra (2001); Shields et al., supra).

In order to enhance CDC and ADCC activity of 2H7, a triple-Ala mutant of the 2H7Fc was constructed. A humanized variant of the anti-HER2 antibody 4D5 has been produced with mutations S298A/E333A/K334A and is known as 4D5Fc110 (i.e., anti-p¹⁸⁵HER2 IgG1 (S298A/E333A/K334A); Shields et al., supra). A plasmid, p4D5Fc110 encoding antibody 4D5Fc110 (Shields et al., supra) was digested with ApaI and HindIII, and the Fc-fragment (containing mutations S298A/E333A/K334A) was ligated into the ApaI/HindIII sites of the 2H7 heavy-chain vector pDR2-v16, to produce pDR2-v31. The amino acid sequence of the version 31 complete H chain is shown in FIG. 8. The L chain is the same as that of v16.

Although the constant domains of the Fc region of IgG1 antibodies are relatively conserved within a given species, allelic variations exist (reviewed by Lefranc and Lefranc, in The Human IgG Subclasses: molecular analysis of structure, function, and regulation, pp. 43-78, F. Shakib (ed.), Pergamon Press, Oxford (1990)).

TABLE 8
Effects of substitutions in the Fc region on CD20 binding. Relative
binding to CD20 was measured in a cell-based (WIL2-S) assay of
framework substitutions. Fc mutations (*) are indicated by EU numbering
(Kabat, supra) and are relative to the 2H7.v16 parent. The combination of
three Ala changes in the Fc region of v.31 is described as “Fc110.” IgG
variants are shown with mutations with respect to the 2H7.v16
background. The relative binding is expressed as the concentration of the
2H7.v6.8 chimera over the concentration of the variant required for
equivalent binding; hence, a ratio <1 indicates weaker affinity for the
variant. Standard deviation in relative affinity determination averaged
+/−10%.

| 2H7 | Fc | Relative |
|---|---|---|
| version | Substitutions* | binding |
| 6.8 | — | -1- |
| 16 | — | 0.65 |
| 31 | S298A, E333A, K334A | 0.62 |

### EXAMPLE 6

### Humanized 2H7 Variants with Enhanced Stability

For development as therapeutic proteins, it is desirable to choose variants that remain stable with respect to oxidation, deamidation, or other processes that may affect product quality, in a suitable formulation buffer. In 2H7.v16, several residues were identified as possible sources of instability: VL (M32) and VH (M34, N100). Therefore, mutations were introduced at these sites for comparison with v16.

TABLE 9
Relative binding of 2H7 variants, designed for enhanced stability and/or effector function, to CD20
in a cell-based (WIL2-S) assay. IgG variants are shown with mutations with respect to the 2H7.v16
background. The relative binding is expressed as the concentration of the 2H7.v6.8 chimera over
the concentration of the variant required for equivalent binding; hence, a ratio <1 indicates weaker
affinity for the variant. Standard deviation in relative affinity determination averaged +/−10%.
Framework substitutions in the variable domains are relative to 2H7.v16 according to the numbering
system of Kabat and Fc mutations (*) are indicated by EU numbering (Kabat et al., supra).

| | Heavy- | Light- | | |
|---|---|---|---|---|
| | chain | chain | | |
| 2H7 | (V_{H}) | (V_{L}) | | Relative |
| version | changes | changes | Fc changes* | binding |
| 6.8 | (chimera) | (chimera) | — | -1- |
| 16 | — | — | — | 0.65 |
| 62 | — | M32I | — | 0.46 |
| 63 | M34I | — | — | 0.49 |
| 64 | N100A | — | — | |
| 65 | N100A | L47W | — | 0.74 |
| 66 | S99A | L47W | — | 0.62 |
| 67 | N54A | — | — | |
| 68 | — | M32I | — | 0.48 |
| 69 | — | M32L | — | 0.52 |
| 70 | N100A | — | S298A, E333A, K334A | 0.80 |
| 71 | N100D | — | S298A, E333A, K334A | 0.44 |
| 72 | N100A | M32I | — | 0.58 |
| 73 | N100A | M32L | — | 0.53 |
| 74 | N100A | M32I | S298A, E333A, K334A | 0.61 |
| 75 | N100A | M32L | S298A, E333A, K334A | 0.60 |
| 113 | — | — | E356D, M358L | 0.60** |
| 114 | D56A, N100A | M32L, S92A | S298A, E333A, K334A | 1.2** |
| 115 | D56A, N100A | M32L, S92A | S298A, E333A, K334A, E356D, M358L | 1.4** |
| 116 | D56A, N100A | M32L, S92A | S298A, K334A, K322A | 1.2** |
| 134 | D56A, N100A | M32L, S92A | E356D, M358L, D265A | 1.5** |
| 135 | D56A, N100A | M32L, S92A | E356D, M358L, D265A, K326W | 0.95** |
| 138 | D56A, N100A | M32L, S92A | S298A, E333A, K334A, K326A | 1.2** |
| 139 | D56A, N100A | M32L, S92A | S298A, E333A, K334A, K326A, E356N, M358L | 1.1** |
| 154 | — | — | D265A | 0.70** |
| 155 | — | — | S298A, K322A, K334A | 0.70** |

**Variants that were measured with 2H7.v16 as comparator; relative binding values are normalized to that of the chimera.

Additional Fc mutations were combined with stability- or affinity-enhancing mutations to alter or enhance effector functions based on previously reported mutations (Idusogie et al. J. Immunol. 164: 4178-4184 (2000); Idusogie et al. J. Immunol. 166:2571-2575 (2001); Shields et al. J. Biol. Chem. 276:6591-6604 (2001)). These changes include S298, E333A, K334A as described in Example 5; K322A to reduce CDC activity; D265A to reduce ADCC activity; K326A or K326W to enhance CDC activity; and E356D/M358L to test the effects of allotypic changes in the Fc region. None of these mutations caused significant differences in CD20 binding affinity.

To test the effects of stability mutations on the rate of protein degradation, 2H7.v16 and 2H7.v73 were and incubated at 12-14 mg/mL in 10 mM histidine, 6% sucrose, 0.02% POLYSORBATE 20™ emulsifier, pH 5.8 and incubated at 40° C. for 16 days. The incubated samples were then assayed for changes in charge variants by ion-exchange chromatography, aggregation, and fragmentation by size-exclusion chromatography, and relative binding by testing in a cell-based (WIL2-S) assay.

The results show that 2H7 v.73 has greater stability compared to 2H7 v.16 with respect to losses in the fraction of main peak by ion-exchange chromatography under accelerated stability conditions. No significant differences were seen with respect to aggregation, fragmentation, or binding affinity.

### EXAMPLE 7

### Scatchard Analysis of Antibody Binding to CD20 on WIL2-S Cells

Equilibrium dissociation constants (K_{d}) were determined for 2H7 IgG variants binding to WIL2-S cells using radiolabeled 2H7 IgG. IgG variants were produced in CHO cells. RITUXAN® (source for all experiments is Genentech, S. San Francisco, CA) and murine 2H7 (BD PharMingen, San Diego, Calif.) were used for comparison with humanized variants. The murine 2H7 antibody is also available from other sources, e.g., eBioscience, and Calbiochem (both of San Diego, Calif.), Accurate Chemical & Scientific Corp., (Westbury, N.Y.), Ancell (Bayport, Minn.), and Vinci-Biochem (Vinci, Italy). All dilutions were performed in binding assay buffer (DMEM media containing 1% bovine serum albumin, 25 mM HEPES pH 7.2, and 0.01% sodium azide). Aliquots (0.025 mL) of ¹²⁵I-2H7.v16 (iodinated with lactoperoxidase) at a concentration of 0.8 nM were dispensed into wells of a V-bottom 96-well microassay plate, and serial dilutions (0.05 mL) of cold antibody were added and mixed. WIL2-S cells (60,000 cells in 0.025 mL) were then added. The plate was sealed and incubated at room temperature for 24 hours, then centrifuged for 15 min at 3,500 RPM. The supernatant was then aspirated and the cell pellet was washed and centrifuged. The supernatant was again aspirated, and the pellets were dissolved in 1N NaOH and transferred to tubes for gamma counting. The data were used for Scatchard analysis (Munson and Rodbard Anal. Biochem. 107:220-239 (1980)) using the program Ligand (McPherson Comput. Programs Biomed. 17:107-114 (1983)). The results, shown in Table 10, indicate that humanized 2H7 variants had similar CD20 binding affinity as compared to murine 2H7, and similar binding affinity to RITUXAN®. It is expected that 2H7.v31 will have very similar K_{d} to v.16 on the basis of the binding shown in Table 8 above.

TABLE 10
Equilibrium binding affinity of 2H7 variants from Scatchard analysis

| Antibody variant | K_{d} (nM) | n |
|---|---|---|
| RITUXAN ® | 0.99 ± 0.49 | 3 |
| 2H7 (murine) | 1.23 ± 0.29 | 3 |
| 2H7.v16 | 0.84 ± 0.37 | 4 |
| 2H7.v73 | 1.22 ± 0.39 | 4 |
| 2H7.v75 | 1.09 ± 0.17 | 4 |

### EXAMPLE 8

### Complement-Dependent Cytotoxicity (CDC) Assays

2H7 IgG variants were assayed for their ability to mediate complement-dependent lysis of WIL2-S cells, a CD20-expressing lymphoblastoid B-cell line, essentially as described (Idusogie et al. J. Immunol. 164:4178-4184 (2000); Idusogie et al. J. Immunol. 166:2571-2575 (2001)). Antibodies were serially diluted 1:3 from a 0.1 mg/mL stock solution. A 0.05 mL aliquot of each dilution was added to a 96-well tissue culture plate that contained 0.05 mL of a solution of normal human complement (Quidel, San Diego, Calif.). To this mixture, 50,000 WIL2-S cells were added in a 0.05 mL volume. After incubation for 2 hours at 37° C., 0.05 mL of a solution of ALAMAR BLUE™ resazurin (Accumed International, Westlake, Ohio) was added, and incubation was continued for an additional 18 hours at 37° C. Covers were then removed from the plates, and they were shaken for 15 min at room temperature on an orbital shaker. Relative fluorescent units (RFU) were read using a 530-nm excitation filter and a 590-nm emission filter. An EC₅₀ was calculated by fitting RFU as a function of concentration for each antibody using KALEIDAGRAPH™ software.

The results (Table 11) show surprising improvement in CDC by humanized 2H7 antibodies, with relative potency similar to RITUXAN® for v.73, 3-fold more potent than RITUXAN® for v.75, and 3-fold weaker than RITUXAN® for v.16.

TABLE 11
CDC activity of 2H7 antibodies compared to RITUXAN ®.
Numbers >1 indicate less potent CDC activity than RITUXAN ®
and numbers <1 indicate more potent activity than RITUXAN ®.
Antibodies were produced from stable CHO lines, except that those
indicated by (*) were produced transiently.

| Antibody variant | n | EC₅₀(variant)/EC₅₀(RITUXAN ®) |
|---|---|---|
| RITUXAN ® | 4 | -1- |
| 2H7.v16 | 4 | 3.72; 4.08 |
| 2H7.v31* | 4 | 2.21 |
| 2H7.v73 | 4 | 1.05 |
| 2H7.v75 | 4 | 0.33 |
| 2H7.v96* | 4 | 0.956 |
| 2H7.v114* | 4 | 0.378 |
| 2H7.v115* | 4 | 0.475 |
| 2H7.v116* | 1 | >100 |
| 2H7.v135* | 2 | 0.42 |

### EXAMPLE 9

### Antibody-Dependent Cellular Cytotoxicity (ADCC) Assays

2H7 IgG variants were assayed for their ability to mediate NK-cell lysis of WIL2-S cells, a CD20-expressing lymphoblastoid B-cell line, essentially as described (Shields et al. J. Biol. Chem. 276:6591-6604 (2001)) using a lactate dehydrogenase (LDH) readout. NK cells were prepared from 100 mL of heparinized blood, diluted with 100 mL of PBS, obtained from normal human donors who had been isotyped for FcγRIII, also known as CD16 (Koene et al. Blood 90:1109-1114 (1997)). In this experiment, the NK cells were from human donors heterozygous for CD16 (F158/V158). The diluted blood was layered over 15 mL of lymphocyte-separation medium (ICN Biochemical, Aurora, Ohio) and centrifuged for 20 min at 2000 RPM. White cells at the interface between layers were dispensed to 4 clean 50-mL tubes, which were filled with RPMI medium containing 15% fetal calf serum. Tubes were centrifuged for 5 min at 1400 RPM and the supernatant was discarded. Pellets were resuspended in MACS buffer (0.5% BSA, 2 mM EDTA), and NK cells were purified using beads (NK Cell Isolation Kit, 130-046-502) according to the manufacturer's protocol (Miltenyi Biotech.). NK cells were diluted in MACS buffer to 2×10⁶ cells/mL.

Serial dilutions of antibody (0.05 mL) in assay medium (F12/DMEM 50:50 without glycine, 1 mM HEPES buffer pH 7.2, Penicillin/Streptomycin (100 units/mL; Gibco), glutamine, and 1% heat-inactivated fetal bovine serum) were added to a 96-well round-bottom tissue-culture plate. WIL2-S cells were diluted in assay buffer to a concentration of 4×10⁵/mL. WIL2-S cells (0.05 mL per well) were mixed with diluted antibody in the 96-well plate and incubated for 30 min at room temperature to allow binding of antibody to CD20 (opsonization).

The ADCC reaction was initiated by adding 0.1 mL of NK cells to each well. In control wells, 2% TRITON® X-100 alkylaryl polyether alcohol was added. The plate was then incubated for 4 hours at 37° C. Levels of LDH released were measured using a cytotoxicity (LDH) detection kit (Kit#1644793, Roche Diagnostics, Indianapolis, Ind.) following the manufacturer's instructions. 0.1 mL of LDH developer was added to each well, followed by mixing for 10 seconds. The plate was then covered with aluminum foil and incubated in the dark at room temperature for 15 min. Optical density at 490 nm was then read and used to calculate % lysis by dividing by the total LDH measured in control wells. Lysis was plotted as a function of antibody concentration, and a 4-parameter curve fit (KALEIDAGRAPH™ software) was used to determine EC₅₀ concentrations.

The results showed that humanized 2H7 antibodies were active in ADCC, with relative potency 20-fold higher than RITUXAN® for v.31 and v.75, 5-fold more potent than RITUXAN® for v.16, and almost 4-fold higher than RITUXAN® for v.73.

TABLE 12
ADCC activity of 2H7 antibodies on WIL2-S cells compared to
2H7.v16, based on n experiments. (Values >1 indicate lower
potency than 2H7.v16, and values <1 indicate greater potency.)

| Antibody variant | n | EC₅₀(variant)/EC₅₀(2H7.v16) |
|---|---|---|
| RITUXAN ® | 4 | 5.3 |
| 2H7.v16 | 5 | 1 |
| 2H7.v31 | 1 | 0.24 |
| 2H7.v73 | 5 | 1.4 |
| 2H7.v75 | 4 | 0.25 |

Additional ADCC assays were carried out to compare combination variants of 2H7 with RITUXAN®. The results of these assays indicated that 2H7.v114 and 2H7.v115 have >10-fold improved ADCC potency as compared to RITUXAN® (Table 13).

TABLE 13
ADCC activity of 2H7 antibodies on WIL2-S cells compared to
RITUXAN ®, based on n experiments (Values >1 indicate
lower potency than RITUXAN ®, and values <1 indicate
greater potency).

| Antibody variant | | EC50(variant)/EC50(RITUXAN ®) |
|---|---|---|
| RITUXAN ® | 2 | -1- |
| 2H7 v.16 | 2 | 0.52 |
| 2H7 v.96 | 2 | 0.58 |
| 2H7.v114 | 2 | 0.093 |
| 2H7.v115 | 2 | 0.083 |
| 2H7.v116 | 2 | 0.30 |

### EXAMPLE 10

### In vivo Effects of 2H7 Variants in a Pilot Study in Cynomolgus Monkeys

2H7 variants, produced by transient transfection of CHO cells, were tested in normal male cynomolgus (Macaca fascicularis) monkeys in order to evaluate their in vivo activities. Other anti-CD20 antibodies, such as C2B8 (RITUXAN®), have demonstrated an ability to deplete B-cells in normal primates (Reff et al. Blood 83: 435-445 (1994)).

In one study, humanized 2H7 variants were compared. In a parallel study, RITUXAN® was also tested in cynomolgus monkeys. Four monkeys were used in each of five dose groups: (1) vehicle, (2) 0.05 mg/kg hu2H7.v16, (3) 10 mg/kg hu2H7.v16, (4) 0.05 mg/kg hu2H7.v31, and (5) 10 mg/kg hu2H7.v31. Antibodies were administered intravenously at a concentration of 0, 0.2, or 20 mg/mL, for a total of two doses, one on day I of the study, and another on day 8. The first day of dosing is designated day 1 and the previous day is designated day -1; the first day of recovery (for 2 animals in each group) is designated as day 11. Blood samples were collected on days -19, -12, 1 (prior to dosing), and at 6 hours, 24 hours, and 72 hours following the first dose. Additional samples were taken on day 8 (prior to dosing), day 10 (prior to sacrifice of 2 animals/group), and on days 36 and 67 (for recovery animals).

Peripheral B-cell concentrations were determined by a FACS method that counted CD3-/CD40+cells. The percent of CD3-CD40+B cells of total lymphocytes in monkey samples was obtained by the following gating strategy. The lymphocyte population was marked on the forward scatter/side scatter scattergram to define Region 1 (R1). Using events in R1, fluorescence intensity dot plots were displayed for CD40 and CD3 markers. Fluorescently labeled isotype controls were used to determine respective cutoff points for CD40 and CD3 positivity.

The results indicated that both 2H7.v16 and 2H7.v31 were capable of producing full peripheral B-cell depletion at the 10 mg/kg dose and partial peripheral B-cell depletion at the 0.05 mg/kg dose. The time course and extent of B-cell depletion measured during the first 72 hours of dosing were similar for the two antibodies. Subsequent analysis of the recovery animals indicated that animals treated with 2H7.v31 showed a prolonged depletion of B-cells as compared to those dosed with 2H7.v16. In particular, for recovery animals treated with 10 mg/kg 2H7.v16, B-cells showed substantial B-cell recovery at some time between sampling on Day 10 and on Day 36. However, for recovery animals treated with 10 mg/kg 2H7.v31, B-cells did not show recovery until some time between Day 36 and Day 67. This suggests a greater duration of full depletion by about one month for 2H7.v31 compared to 2H7.v16.

No toxicity was observed in the monkey study at low or high dose and the gross pathology was normal. In other studies, v 16 was well tolerated up to the highest dose evaluated of (100 mg/kgx2=1200 mg/m²×2) following i.v. administration of 2 doses given 2 weeks apart in these monkeys.

Data in Cynomolgus monkeys with 2H7.v16 versus RITUXAN® suggest that a 5-fold reduction in CDC activity does not adversely affect potency. An antibody with potent ADCC activity but reduced CDC activity may have a more favorable safety profile with regard to first infusion reactions than one with greater CDC activity.

### EXAMPLE 11

### Fucose-Deficient 2H7 Variant Antibodies with Enhanced Effector Function

Normal CHO and HEK293 cells add fucose to IgG oligosaccharide to a high degree (97-98%). IgG from sera are also highly fucosylated.

DP12, a dihydrofolate-reductase-minus (DHFR) CHO cell line that is fucosylation competent, and Lec13, a cell line that is deficient in protein fucosylation, were used to produce antibodies for this study. The CHO cell line, Pro-Lec13.6a (Lec13), was obtained from Professor Pamela Stanley of Albert Einstein College of Medicine of Yeshiva University. Parental lines are Pro-(proline auxotroph) and Gat-(glycine, adenosine, thymidine auxotroph). The CHO-DP12 cell line is a derivative of the CHO-K1 cell line (ATCC #CCL-61), which is dihydrofolate reductase deficient, and has a reduced requirement for insulin. Cell lines were transfected with cDNA using the SUPERFECT™ transfection reagent method (Qiagen, Valencia, Calif.). Selection of the Lec13 cells expressing transfected antibodies was performed using puromycin dihydrochloride (Calbiochem, San Diego, Calif.) at 10 μg/ml in growth medium containing: MEM Alpha Medium with L-glutamine, ribonucleosides and deoxyribonucleosides (GIBCO-BRL, Gaithersburg, Md.), supplemented with 10% inactivated FBS (Gibco), 10 mM HEPES, and 1×penicillin/streptomycin (Gibco). The CHO cells were similarly selected in growth medium containing Ham's F12 without GHT: Low Glucose DMEM without Glycine with NaHCO₃ supplemented with 5% FBS (Gibco), 10 mM HEPES, 2 mM L-glutamine, 1×GHT (glycine, hypoxanthine, thymidine), and 1×penicillin/streptomycin.

Colonies formed within two to three weeks and were pooled for expansion and protein expression. The cell pools were seeded initially at 3×10⁶ cells/10 cm plate for small batch protein expression. The cells were converted to serum-free media once they grew to 90-95% confluency, and after 3-5 days cell supernatants were collected and tested in an Fc IgG- and intact IgG-ELISA to estimate protein expression levels. Lec 13 and CHO cells were seeded at approximately 8×10⁶ cells/15-cm plate one day prior to converting to PS24 production medium, supplemented with 10 mg/L recombinant human insulin and 1 mg/L trace elements.

Lec13 cells and DP12 cells remained in serum-free production medium for 3-5 days. Supernatants were collected and clarified by centrifugation in 150-ml conical tubes to remove cells and debris. The protease inhibitors PMSF and aprotinin (Sigma, St. Louis, Mo.) were added and the supernatants were concentrated 5-fold on stirred cells using MWCO30™ filters (Amicon, Beverly, Mass.) prior to immediate purification using protein G chromatography (Amersham Pharmacia Biotech, Piscataway, N.J.)). All proteins were buffer exchanged into PBS using CENTRIPREP-30™ concentrators (Amicon) and analyzed by SDS-polyacrylamide gel electrophoresis. Protein concentrations were determined using A280 absorbance values and verified using amino acid composition analysis.

The CHO cells were transfected with vectors expressing humanized 2H7v16, 2H7v.31 and selected as described. The 2H7v.16 antibody retains the wild-type Fc region, while v.31 (see Example 5, Table 8 above) has an Fc region wherein 3 amino acid changes were made (S298A, E333A, K334A), which results in higher affinity for the FcγRIIIa receptor (Shields et al. J. Biol. Chem. 276 (9):6591-6604 (2001)). Following transfection and selection, individual colonies of cells were isolated and evaluated for protein expression level, and the highest producers were subjected to methotrexate selection to select for cells that had amplified the plasmid copy number and that, therefore, produced higher levels of antibody. Cells were grown and transferred to serum-free medium for a period of 7 days, then the medium was collected and loaded onto a protein A column and the antibody was eluted using standard techniques. The final concentration of the antibody was determined using an ELISA that measures intact antibody. All proteins were buffer exchanged into PBS using CENTRIPREP-30™ concentrators. (Amicon) and analyzed by SDS-polyacrylamide gel electrophoresis.

Matrix-Assisted Laser Desorption/Ionization Time-of-Flight (MALDI-TOF) Mass Spectral Analysis of Asparagine-Linked Oligosaccharides.

N-linked oligosaccharides were released from recombinant glycoproteins using the procedure of Papac et al. Glycobiology 8:445-454 (1998). Briefly, the wells of a 96-well polyvinylidine difluoride (PVDF)-lined microtitre plate (Millipore, Bedford, Mass.) were conditioned with 100 μl methanol that was drawn through the PVDF membranes by applying vacuum to the Millipore MULTISCREEN™ vacuum manifold. The conditioned PVDF membranes were washed with 3×250 μl water. Between all wash steps the wells were drained completely by applying gentle vacuum to the manifold. The membranes were washed with reduction and carboxymethylation buffer (RCM) consisting of 6 M guanidine hydrochloride, 360 mM TRIS, 2 mM EDTA, pH 8.6. Glycoprotein samples (50 μg) were applied to individual wells, again drawn through the PVDF membranes by gentle vacuum and the wells were washed with 2×50 μl of RCM buffer. The immobilized samples were reduced by adding 50 μl of a 0.1 M dithiothreitol (DTT) solution to each well and incubating the microtitre plate at 37° C. for 1 hr. DTT was removed by vacuum and the wells were washed with 4×250 μl water.

Cysteine residues were carboxylmethylated by the addition of 50 μl of a 0.1 M iodoacetic acid (IAA) solution that was freshly prepared in 1 M NaOH and diluted to 0.1 M with RCM buffer. Carboxymethylation was accomplished by incubation for 30 min in the dark at ambient temperature. Vacuum was applied to the plate to remove the IAA solution and the wells were washed with 4×250 μl purified water. The PVDF membranes were blocked by the addition of 100 μl of 1% PVP-360 (polyvinylpyrrolidine 360,000 MW) (Sigma) solution and incubation for 1 hr at ambient temperature. The PVP-360 solution was removed by gentle vacuum and the wells were washed 4×250 μl water. The PNGASE F™ amidase (New England Biolabs, Beverly, Mass.) digest solution, 25 μl of a 25 unit/ml solution in 10 mM TRIS acetate, pH 8.4, was added to each well and the digest proceeded for 3 hr at 37° C. After digestion, the samples were transferred to 500 μl Eppendorf tubes and 2.5 μL of a 1.5 M acetic acid solution was added to each sample. The acidified samples were incubated for 3 hr at ambient temperature to convert the oligosaccharides from glycosylamines to the hydroxyl form. Prior to MALDI-TOF mass spectral analysis, the released oligosaccharides were desalted using a 0.7-ml bed of cation-exchange resin (AG50W-X8™ resin in the hydrogen form) (Bio-Rad, Hercules, Calif.) slurried packed into compact-reaction tubes (US Biochemical, Cleveland, Ohio).

For MALDI-TOF mass spectral analysis of the samples in the positive mode, the desalted oligosaccharides (0.5 μl aliquots) were applied to the stainless target with 0.5 μl of the 2,5 dihydroxybenzoic acid matrix (sDHB) that was prepared by dissolving 2 mg 2,5 dihydroxybenzoic acid with 0.1 mg of 5-methoxyslicylic acid in 1 ml of ethanol/O mM sodium chloride 1:1 (v/v). The sample/matrix mixture was dried by vacuum. For analysis in the negative mode, the desalted N-linked oligosaccharides (0.5 μl aliquots) were applied to the stainless target along with 0.5 μl 2′,4′,6′-trihydroxyacetophenone matrix (THAP) prepared in 1:3 (v/v) acetonitrile/13.3 mM ammonium citrate buffer. The sample/matrix mixture was vacuum dried and then allowed to absorb atmospheric moisture prior to analysis. Released oligosaccharides were analyzed by MALDI-TOF on a PERSEPTIVE BIOSYSTEMS™ VOYAGER-DE™ mass spectrometer. The mass spectrometer was operated at 20 kV either in the positive or negative mode with the linear configuration and utilizing delayed extraction. Data were acquired using a laser power of 1300 and in the data summation mode (240 scans) to improve the signal-to-noise ratio. The instrument was calibrated with a mixture of standard oligosaccharides and the data were smoothed using a 19-point Savitsky-Golay algorithm before the masses were assigned. Integration of the mass spectral data was achieved using the CAESAR 7.0™ data analysis software package (SciBridge Software). NK cell ADCCs.

ADCC assays were performed as described in Example 9. NK-to-target cell (WIL2-S) ratio was 4 to 1, assays were run for 4 hours, and toxicity was measured as before using the lactose dehydrogenase assay. Target cells were opsonized with the concentrations of antibody indicated for 30 min prior to addition of NK cells. The RITUXAN® antibody used was from Genentech (S. San Francisco, Calif.).

The results show that underfucosylated antibodies mediate NK-cell target-cell killing more efficiently than do antibodies with a full complement of fucose. The underfucosylated antibody, 2H7v.31, is most efficient at mediating target-cell killing. This antibody is effective at lower concentrations and is capable of mediating killing of a greater percentage of target cells at higher concentrations than are the other antibodies. The activity of the antibodies is as follows: Lec13-derived 2H7 v31>Lec 13 derived 2H7v16>Dp12 derived 2H7v31>Dp12 derived 2H7v16> or =to RITUXAN®. The protein and carbohydrate alterations are additive. Comparison of the carbohydrate found on native IgG from the Lec13-produced and CHO-produced IgG showed no appreciable differences in the extent of galactosylation, and hence the results can be attributed solely to the presence/absence of fucose.

### EXAMPLE 12

### Cloning of Cynomolgus Monkey CD20 and Antibody Binding

The CD20 DNA sequence for cynomolgus monkey (Macaca fascicularis) was determined upon the isolation of cDNA encoding CD20 from a cynomolgus spleen cDNA library. A SUPERSCRIPT™ Plasmid System for cDNA Synthesis and Plasmid Cloning (Cat#18248-013, Invitrogen, Carlsbad, Calif.) was used with slight modifications to construct the library. The cDNA library was ligated into a pRK5E vector using restriction sites XhoI and NotI. mRNA was isolated from spleen tissue ((California Regional Research Primate Center, Davis, Calif.). Primers to amplify cDNA encoding CD20 were designed based on non-coding sequences of human CD20. N-terminal region primer 5′-AGTTTTGAGAGCAAAATG-3′ (SEQ ID NO:41) and C-terminal region primer 5′-AAGCTATGAACACTAATG-3′(SEQ ID NO:42) were used to clone by polymerase chain reaction (PCR) the cDNA encoding cynomolgus monkey CD20. The PCR reaction was carried out using the PLATINUM TAQ DNA POLYMERASE HIGH FIDELITY™ system according to the manufacturer's recommendation (Gibco, Rockville, Md.). The PCR product was subcloned into PCR®2.1-TOPO® vector (Invitrogen) and transformed into XL-1 blue E. coli (Stratagene. La Jolla, Calif.). Plasmid DNA containing ligated PCR products was isolated from individual clones and sequenced.

The amino acid sequence for cynomolgus monkey CD20 is shown in FIG. 19. FIG. 20 shows a comparison of cynomolgus and human CD20. The cynomolgus monkey CD20 is 97.3% similar to human CD20 with 8 differences. The extracellular domain contains one change at V157A, while the remaining 7 residues can be found in the cytoplasmic or transmembrane regions.

Antibodies directed against human CD20 were assayed for the ability to bind and displace FITC-conjugated murine 2H7 binding to cynomolgus monkey cells expressing CD20. Twenty milliliters of blood were drawn from 2 cynomolgus monkeys (California Regional Research Primate Center, Davis, Calif.) into sodium heparin and shipped directly to Genentech, Inc. On the same day, the blood samples were pooled and diluted 1:1 by the addition of 40 ml of PBS. 20 ml of diluted blood was layered on 4×20 ml of FICOLL-PAQUE™ PLUS (Amersham Biosciences, Uppsala, Sweden) in 50 ml conical tubes (Cat#352098, Falcon, Franklin Lakes, N.J.) and centrifuged at 1300 rpm for 30 minutes room temperature in a SORVAL™7 centrifuge (Dupont, Newtown, Conn.). The PBMC layer was isolated and washed in PBS. Red blood cells were lysed in a 0.2% NaCl solution, restored to isotonicity with an equivalent volume of a 1.6% NaCl solution, and centrifuged for 10 minutes at 1000 RPM. The PBMC pellet was resuspended in RPMI 1640 (Gibco, Rockville, Md.) containing 5% FBS and dispensed into a 10-cm tissue culture dish for 1 hour at 37° C. The non-adherent B- and T-cell populations were removed by aspiration, centrifuged, and counted. A total of 2.4×10⁷ cells were recovered. The resuspended PBMC were distributed into twenty 12×75-mm culture tubes (Cat#352053, Falcon), with each tube containing 1×10⁶ cells in a volume of 0.25 ml. Tubes were divided into four sets of five tubes. To each set was added either media (RPMI1640, 5% FBS), titrated amounts of control human IgG₁ antibody, RITUXAN®, 2H7.v16, or 2H7.v31. The final concentration of each antibody was 30, 10, 3.3 and 1.1 nM. In addition, each tube also received 20 μl of fluorescein isothiocyanate (FITC)-conjugated anti-human CD20 (Cat#555622, BD Biosciences, San Diego, Calif.). The cells were gently mixed, incubated for 1 hour on ice, and then washed twice in cold PBS. The cell surface staining was analyzed on an EPIC XL-MCL™ flow cytometer (Coulter, Miami, Fla.), and the geometric means derived and plotted (KALEIDAGRAPH™, Synergy Software, Reading, Pa.) versus antibody concentrations.

Data showed that 2H7 v.16 and 2H7 v.31 competitively displaced FITC-murine 2H7 binding to cynomolgus monkey cells. Furthermore, RITUXAN® also displaced FITC-murine 2H7 binding, thus demonstrating that both 2H7 and RITUXAN® bind to an overlapping epitope on CD20. In addition, the data show that the IC₅₀ values for 2H7 v.16, 2H7 v.31 and RITUXAN® are similar and fall in the 4-6 nM range.

### EXAMPLE 13

### Phase I/II Study of rhuMAb 2H7 (2H7.v16) in Moderate-to-Severe Rheumatoid Arthritis

Protocol Synopsis

A randomized, placebo-controlled, multicenter, blinded phase I/II study of the safety of escalating doses of PRO70769 (rhuMAb 2H7) in subjects with moderate-to-severe rheumatoid arthritis receiving stable doses of concomitant methotrexate (MTX).

Objectives

The primary objective of this study is to evaluate the safety and tolerability of escalating intravenous (IV) doses of PRO70769 (rhuMAb 2H7) in subjects with moderate-to-severe rheumatoid arthritis (RA).

Study Design

This is a randomized, placebo-controlled, multicenter, blinded Phase I/II, investigator- and subject-blinded study of the safety of escalating doses of PRO70769 in combination with MTX in subjects with moderate-to-severe RA. The study consists of a dose-escalation phase and a second phase with enrollment of a larger number of subjects. The Sponsor will remain unblended to treatment assignment.

Subjects with moderate-to-severe RA who have failed one to five disease-modifying anti-rheumatic drugs or biologics who currently have unsatisfactory clinical responses to treatment with MTX will be enrolled.

Subjects will be required to receive MTX in the range of 10-25 mg weekly for at least 12 weeks prior to study entry and to be on a stable dose for at least 4 weeks before receiving their initial dose of study drug (PRO70769 or placebo). Subjects may also receive stable doses of oral corticosteroids (up to 10 mg daily or prednisone equivalent) and stable doses of non-steroidal anti-inflammatory drugs (NSAIDs). Subjects will receive two IV infusions of PRO70769 or placebo equivalent at the indicated dose on Days 1 and 15 according to the following dose-escalation plan.

Dose escalation will occur according to specific criteria and after review of safety data by an internal safety data review committee and assessment of acute toxicity 72 hours following the second infusion in the last subject treated in each cohort. After the dose-escalation phase, 40 additional subjects (32 active and 8 placebo) will be randomized to each of the following dose levels: 2×50 mg, 2×200 mg, 2×500 mg, and 2×1000 mg, if the dose levels have been demonstrated to be tolerable during the dose-escalation phase. Approximately 205 subjects will be enrolled in the study.

B-cell counts will be obtained and recorded. B-cell counts will be evaluated using flow cytometry in a 48-week follow-up period beyond the 6-month efficacy evaluation. B-cell depletion will not be considered a dose-limiting toxicity (DLC), but rather the expected pharmacodynamic outcome of PRO70769 treatment.

In an optional substudy, blood for serum and RNA analyses, as well as urine samples, will be obtained from subjects at various timepoints. These samples may be used to identify biomarkers that may be predictive of response to PRO70769 treatment in subjects with moderate-to-severe RA.

Outcome Measures

The primary outcome measure for this study is the safety and tolerability of PRO70769 in subjects with moderate-to-severe RA.

Study Treatment

Cohorts of subjects will receive two IV infusions of PRO70769 or placebo equivalent at the indicated dose on Days 1 and 15 according to the following escalation plan:
10 mg PRO70769 or placebo equivalent: 4 subjects active drug, I control
50 mg PRO70769 or placebo equivalent: 8 subjects active drug, 2 control
200 mg PRO70769 or placebo equivalent: 8 subjects active drug, 2 control
500 mg PRO70769 or placebo equivalent: 8 subjects active drug, 2 control
1000 mg PRO70769 or placebo equivalent: 8 subjects active drug, 2 control
   Efficacy

The efficacy of PRO70769 will be measured by ACR responses. The percentage of subjects who achieve an ACR20, ACR50, and ACR70 response will be summarized by treatment group and 95% confidence intervals will be generated for each group. The components of these responses and their change from baseline will be summarized by treatment and visit.

### CONCLUSION OF EXAMPLES 1-13

The data above demonstrated the success in producing humanized CD20 binding antibodies, in particular, humanized 2H7 antibody variants, that maintained and even enhanced their biological properties. The humanized 2H7 antibodies of the invention bound to CD20 at affinities similar to the murine donor and chimeric 2H7 antibodies and were effective at B-cell killing in a primate, leading to B-cell depletion. Certain variants showed enhanced ADCC over a chimeric anti-CD20 antibody currently used to treat non-Hodgkin's lymphoma (NHL), favoring the use of lower doses of the therapeutic antibody in patients. Additional, whereas it may be necessary for a chimeric antibody that has murine FR residues to be administered at a dose effective to achieve complete B-cell depletion to obviate an antibody response against it, the present humanized antibodies can be administered at dosages that achieve partial or complete B-cell depletion, and for different durations of time, as desired for the particular disease and patient. In addition, these antibodies demonstrated stability in solution. These properties of the humanized 2H7 antibodies make them ideal for use as immunotherapeutic agents in the treatment of CD20-positive autoimmune diseases; these antibodies are not expected to be immunogenic or will at least be less immunogenic than fully murine or chimeric anti-CD20 antibodies in human patients.

### EXAMPLE 14

### Preparation of Further Humanized Antibodies

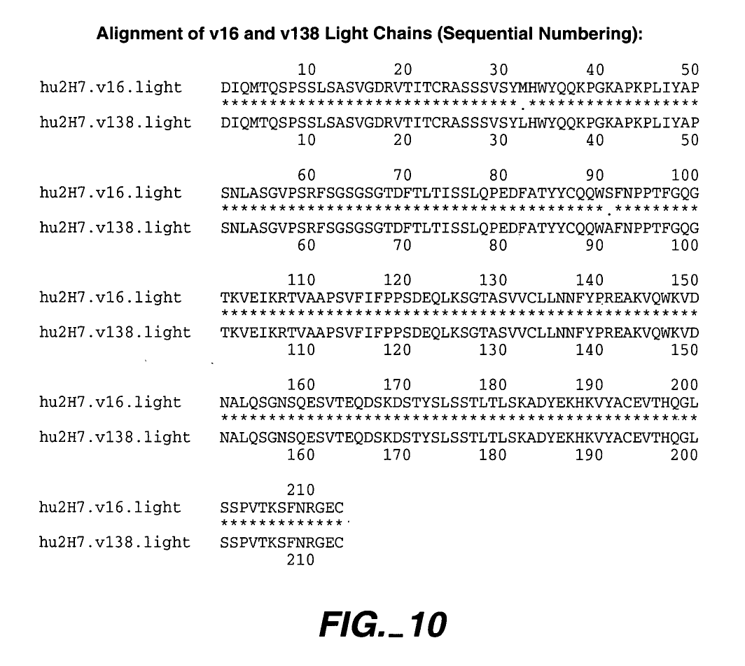

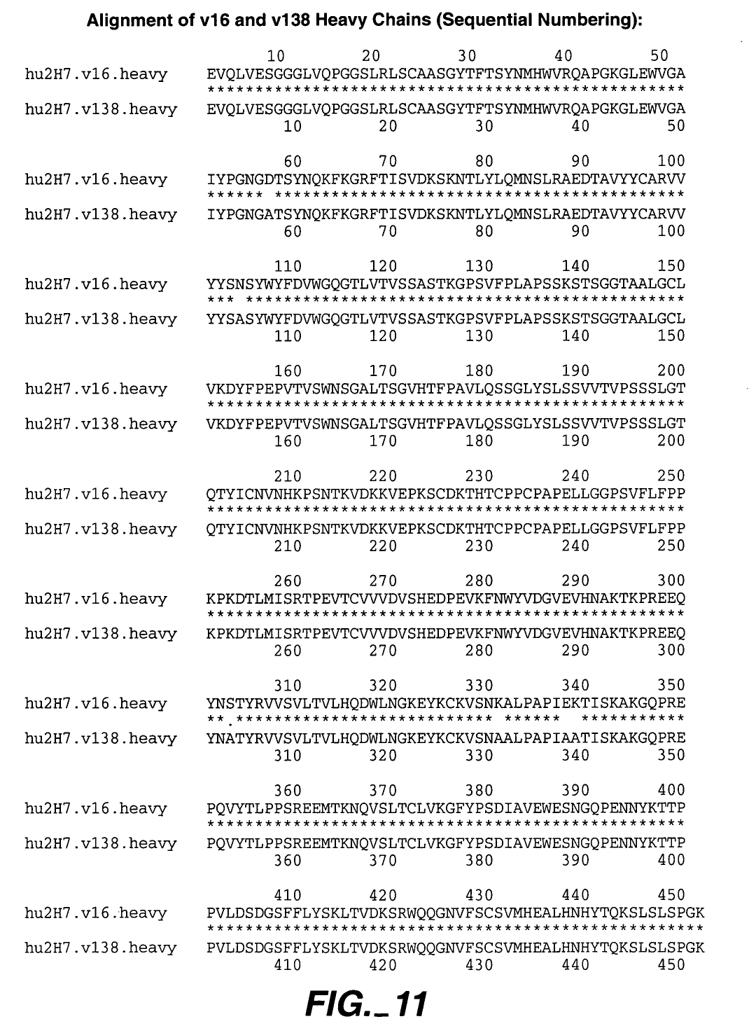

The antibody 2H7.v31 comprising the light- and heavy-chain amino acid sequences of SEQ ID NOS:24 and 28, respectively, may further comprise at least one amino acid substitution in the Fc region that improves ADCC and/or CDC activity, such as one wherein the amino acid substitutions are S298A/E333A/K334A, more preferably 2H7.v31 having the heavy-chain amino acid sequence of SEQ ID NO:28. The antibody may be 2H7.v138 comprising the light- and heavy-chain amino acid sequences of SEQ ID NOS:29 and 30, respectively, as shown in FIGS. 10 and 11, respectively, which are alignments of such sequences with the corresponding light- and heavy-chain amino acid sequences of 2H7.v16. Alternatively, such preferred intact humanized 2H7 antibody is 2H7.v477, which has the light- and heavy-chain sequences of 2H7.v138 except for the amino-acid substitution of N434W. Any of these antibodies may further comprise at least one amino acid substitution in the Fc region that decreases CDC activity, for example, comprising at least the substitution K322A. See U.S. Pat. No. 6,528,624B1 (Idusogie et al.).

Some preferred humanized 2H7 variants are those having the variable light-chain domain of SEQ ID NO:2 and the variable heavy-chain domain of SEQ ID NO:8, i.e., those with or without substitutions in the Fc region, and those having a variable heavy-chain domain with alteration N100A or D56A and N100A in SEQ ID NO:8 and a variable light-chain domain with alteration M32L, or S92A, or M32L and S92A in SEQ ID NO:2, i.e., those with or without substitutions in the Fc region. If substitutions are made in the Fc region, they are preferably one of those set forth in the table below.

In a summary of some various preferred embodiments of the invention, the V region of variants based on the 2H7 version 16 will have the amino acid sequences of v16 except at the positions of amino acid substitutions that are indicated in the table below. Unless otherwise indicated, the 2H7 variants will have the same L chain as that of v16.

| 2H7 | Heavy chain | Light chain | |
|---|---|---|---|
| version | (V_{H}) changes | (V_{L}) changes | Fc changes |
| 16 | | | — |
| 31 | — | — | S298A, E333A, K334A |
| 73 | N100A | M32L | |
| 75 | N100A | M32L | S298A, E333A, K334A |
| 96 | D56A, N100A | S92A | |
| 114 | D56A, N100A | M32L, S92A | S298A, E333A, K334A |
| 115 | D56A, N100A | M32L, S92A | S298A, E333A, K334A, E356D, |
| | | | M358L |
| 116 | D56A, N100A | M32L, S92A | S298A, K334A, K322A |
| 138 | D56A, N100A | M32L, S92A | S298A, E333A, K334A, K326A |
| 477 | D56A, N100A | M32L, S92A | S298A, E333A, K334A, |
| | | | K326A, N434W |
| 375 | — | — | K334L |

In addition to the variants above, the intact humanized 2H7 antibody may be version 138, which comprises the light-chain amino acid sequence:

(SEQ ID NO:29)

DIQMTQSPSSLSASVGDRVTITCRASSSVSYLHWYQQKPGKAPKPLIYAP
SNLASGVPSRFSGSGSGTDFTLTISSLQPEDFATYYCQQWAFNPPTFGQG
TKVEIKRTVAAPSVFIFPPSDEQLKSGTASVVCLLNNFYPREAKVQWKVD
NALQSGNSQESVTEQDSKDSTYSLSSTLTLSKADYEKHKVYACEVTHQGL
SSPVTKSFNRGEC

and the heavy-chain amino acid sequence:

(SEQ ID NO:30)

EVQLVESGGGLVQPGGSLRLSCAASGYTFTSYNMHWVRQAPGKGLEWVGA
IYPGNGATSYNQKFKGRFTISVDKSKNTLYLQMNSLRAEDTAVYYCARVV
YYSASYWYFDVWGQGTLVTVSSASTKGPSVFPLAPSSKSTSGGTAALGCL
VKDYFPEPVTVSWNSGALTSGVHTFPAVLQSSGLYSLSSVVTVPSSSLGT
QTYICNVNHKPSNTKVDKKVEPKSCDKTHTCPPCPAPELLGGPSVFLFPP
KPKDTLMISRTPEVTCVVVDVSHEDPEVKFNWYVDGVEVHNAKTKPREEQ
YNATYRVVSVLTVLHQDWLNGKEYKCKVSNAALPAPIAATISKAKGQPRE
PQVYTLPPSREEMTKNQVSLTCLVKGFYPSDIAVEWESNGQPENNYKTTP
PVLDSDGSFFLYSKLTVDKSRWQQGNVFSCSVMHEALHNHYTQKSLSLSP
GK.

In another embodiment, the humanized 2H7 antibody may comprise the light-chain variable region (V_{L}) sequence of SEQ ID NO:43 and the heavy-chain variable region (V_{H}) sequence of SEQ ID NO:8, wherein the antibody further contains an amino acid substitution of D56A in VH-CDR2, and N100 in VH-CDR3 is substituted with Y or W, wherein SEQ ID NO:43 has the sequence:

(SEQ ID NO:43)

DIQMTQSPSSLSASVGDRVTITCRASSSVSYLHWYQQKPGKAPKPLIYAP
SNLASGVPSRFSGSGSGTDFTLTISSLQPEDFATYYCQQWAFNPPTFGQG
TKVEIKR.

In one embodiment of this lattermost humanized 2H7 antibody, N100 is substituted with Y. In another embodiment, N100 is substituted with W. Moreover, in a further embodiment, the antibody comprises the substitution S100aR in VH-CDR3, preferably further comprising at least one amino acid substitution in the Fc region that improves ADCC and/or CDC activity, such as one that comprises an IgG 1 Fc comprising the amino acid substitutions S298A, E333A, K334A, K326A. Alternatively, the antibody comprises the substitution S100aR in VH-CDR3, preferably further comprising at least one amino acid substitution in the Fc region that improves ADCC but decreases CDC activity, such as one that comprises at least the amino acid substitution K322A, as well as one that further comprises the amino acid substitutions S298A, E333A, K334A.

In one especially preferred embodiment, the antibody is version 511 and comprises the 2H7.v511 light-chain sequence:

(SEQ ID NO:44)

DIQMTQSPSSLSASVGDRVTITCRASSSVSYLHWYQQKPGKAPKPLIYA
PSNLASGVPSRFSGSGSGTDFTLTISSLQPEDFATYYCQQWAFNPPTFG
QGTKVEIKRTVAAPSVFIFPPSDEQLKSGTASVVCLLNNFYPREAKVQW
KVDNALQSGNSQESVTEQDSKDSTYSLSSTLTLSKADYEKHKVYACEVT
HQGLSSPVTKSFNRGEC

and the 2H7.v511 heavy-chain sequence:

(SEQ ID NO:45)

EVQLVESGGGLVQPGGSLRLSCAASGYTFTSYNMHWVRQAPGKGLEWVGA
IYPGNGATSYNQKFKGRFTISVDKSKNTLYLQMNSLRAEDTAVYYCARVV
YYSYRYWYFDVWGQGTLVTVSSASTKGPSVFPLAPSSKSTSGGTAALGCL
VKDYFPEPVTVSWNSGALTSGVHTFPAVLQSSGLYSLSSVVTVPSSSLGT
QTYICNVNHKPSNTKVDKKVEPKSCDKTHTCPPCPAPELLGGPSVFLFPP
KPKDTLMISRTPEVTCVVVDVSHEDPEVKFNWYVDGVEVHNAKTKPREEQ
YNATYRVVSVLTVLHQDWLNGKEYKCKVSNAALPAPIAATISKAKGQPRE
PQVYTLPPSREEMTKNQVSLTCLVKGFYPSDIAVEWESNGQPENNYKTTP
PVLDSDGSFFLYSKLTVDKSRWQQGNVFSCSVMHEALHNHYTQKSLSLSP
GK.

### EXAMPLE 15

### Clinical Study of Rituximab in Polychondritis

Patients diagnosed with polychondritis are treated with RITUXAN® antibody. The patient treated will not have a B-cell malignancy.

RITUXAN® is administered intravenously (IV) to the patient according to any of the following dosing schedules:
(A) 50 mg/m² IV day 1
   150 mg/m² IV on days 8, 15 & 22
(B) 150 mg/m² IV day 1
   375 mg/m² IV on days 8, 15 & 22
(C) 375 mg/m² IV days 1, 8, 15 & 22

Further adjunct therapies (such as immunosuppressive agents as noted above) may be combined with the RITUXAN® therapy, but preferably the patient is treated with RITUXAN® as a single agent throughout the course of therapy.

Overall response rate is determined based upon a reduction in inflammation of cartilaginous tissues as determined by standard chemical parameters. Administration of RITUXAN® will improve any one or more of the symptoms of polychondritis in the patient treated as described above.

### EXAMPLE 16

### Clinical Study of Rituximab in Mononeuritis Multiplex

Patients with clinical diagnosis of mononeuritis multiplex as defined herein are treated with rituximab (RITUXAN®) antibody, optionally in combination with steroid therapy. The patient treated will not have a B-cell malignancy. A detailed and complete medical history is vitally important in determining the possible underlying cause of the disorder. Pain often begins in the low back or hip and spreads to the thigh and knee on one side. The pain usually is characterized as deep and aching with superimposed lancinating jabs that are most severe at night. Individuals with diabetes typically present with acute onset of unilateral severe thigh pain that is followed rapidly by weakness and atrophy of the anterior thigh muscles and loss of the knee reflex. Other possible symptoms that may be reported by the patient include the following: numbness, tingling, abnormal sensation, burning pain—dysesthesia, difficulty moving a body part—paralysis, lack of controlled movement of a body part. Loss of sensation and movement may be associated with dysfunction of specific nerves. Examination reveals preservation of reflexes and good strength except in regions more profoundly affected. Some common findings of mononeuritis multiplex may include the following (not listed in order of frequency): sciatic nerve dysfunction, femoral nerve dysfunction, common peroneal nerve dysfunction, auxiliary nerve dysfunction, radial nerve dysfunction, median nerve dysfunction, ulnar nerve dysfunction, and autonomic dysfunction, i.e., the part of the nervous system that controls involuntary bodily functions, such as the glands and the heart.

A positive response to therapy is projected as improvement in two of the four parameters listed below to account for this variance and also based upon previous treatment studies in diabetic neuropathy (Jaradeh et al. Journal of Neurology, Neurosurgery and Psychiatry 67:607-612 (1999)). Patients must have measurable neuropathy as defined by electrophysiologic testing. Patients with known diabetic or hereditary neuropathy are excluded.

The patients must have adequate organ function as measured by the following criteria (values should be obtained within 2 months prior to registration): Hepatic: AST<3×upper limit of lab normal and bilirubin<2.0 mg/dl. Renal: Creatinine<3.0 mg/dl.

Rituximab will be administered in an out-patient setting intravenously. An in-line filter is not required. The initial rate is 50 mg/hr for the first hour. If no toxicity is seen, the rate may be escalated gradually in 50 mg/hr increments at 30-minute intervals to a maximum of 400 mg/hr. If the first dose is well tolerated, the initial rate for subsequent dose is 100 mg/hr, increased gradually in 100 mg/hr increments at 30-minute intervals, not to exceed 400 mg/hr. If the patient experiences fever and rigors, the antibody infusion is discontinued. The severity of the side effects should be evaluated. If the symptoms improve, the infusion is continued initially at one-half the previous rate. Following the antibody infusion, the intravenous line should be maintained for medications as needed. If there are no complications after one hour of observation, the intravenous line may be discontinued.

All patients registered to this study will receive rituximab weekly for 4 consecutive weeks. The dose is based on actual surface area. The administration schedule is rituximab: 375 mg/m2 weekly×4 by IV infusion on day 1, 8, 15 and 22. All patients should be premedicated with 650 mg of TYLENOL® pain reliever and 50 mg of BENADRYL® allergic medication given IV or PO to reduce adverse events 30-60 minutes prior to treatment. Medications for the treatment of hypersensitivity reactions, e.g. epinephrine, antihistamines and corticosteroids, should be available for immediate use in the event of a reaction during administration. In addition, an anti-pain agent such as acetaminophen, aspirin, amitriptyline (ELAVIL®), carbamazepine (TEGRETOL®), phenyltoin (DILANTIN®), gabapentin (NEURONTIN®), (E)-N-Vanillyl-8-methyl-6-noneamid (CAPSAICIN®), or a nerve blocker may be employed in conjunction with the rituximab.

Neuropathy will be evaluated by several different parameters: 1) EMG/NCS 2) Quantitative Sensory Testing 3) Neuropathy Impairment Score 4) Neuropathy Symptoms and Change Questionnaire.

EMG/NCS: Electromyography and nerve conduction velocity measurements will be performed at three, six and twelve months post-infusion of rituximab by the same electromyographer and technician. Summary data from each study will be used for comparison with initial values including mean sensory nerve action potential (sural, median and ulnar), mean compound motor nerve action potential (peroneal at the anterior tibialis, tibial, ulnar and median), and mean conduction velocity of motor nerves. Mean F wave latencies and proximal-to-distal motor amplitude ratios will also be calculated. An objective response would require ≧10% improvement from base line. Stable disease would indicate no significant change in neuropathy (+/−≧10). Progressive disease would indicate worsening of neuropathy (>10% from baseline).

Quantitative Sensory Testing: Quantitative sensory test with vibration detection threshold (VDT), cooling detection threshold (CDT), and heat pain threshold (HPT) on the dorsum of the foot and hand in addition to sudomotor axon reflex test (QSART) of the distal foot and hand will be performed at three, six and twelve months post-infusion of rituximab by the same technician. Abnormalities in these tests can be transformed into points based on the percentile score in relationship to standard deviation. A change of two percentiles from the pre-study measurements will be considered significant.

Neuropathy Impairment Score (NIS): This test measures reflexes, sensation and muscle strength. A functional assessment of the lower limbs with walking on toes, heels and arising from a kneeled position is made. A score will be performed at three, six and twelve months post-infusion of rituximab by the same neurologist throughout the study. Improvement will be defined as a decrease in NIS by 5 points or more (Dyck “Quantitating severity of neuropathy” In: Dyck et al. Eds. Peripheral Neuropathy. Philadelphia: W B Saunders, 686-697 (1993)).

Neuropathy Symptoms and Change Questionnaire (NSC): This questionnaire consists of 38 items answered in a true or false fashion. It evaluates for the presence or absence of neuropathic symptoms, their severity and change over time. It will be performed by the same neurologist for each patient throughout the study. A change of 10% from baseline score will be considered significant.

The primary outcome measure of the study is patient improvement. A patient is classified as improving if he/she shows significant improvement on 2 of the 4 parameters listed above, while he/she does not decline on any of the other measures. Based on this response classification, exact 95% confidence intervals are computed for the response rates based on a binomial calculation. With 14 patients the width of this interval will be less than about 50% if the true response rate is between 30-70%, about 40% if the rate is between 70-90% or 10-30%, and about 30% if the rate is >90% or <10%.

Point estimates and 95% confidence intervals will be computed for the proportion of patients with a successful outcome on each parameter using exact binomial intervals. For each continuous or ordinal measurement, exact 95% confidence intervals will be computed for the change from baseline by the Hodges-Lehmann statistic and the Tukey Interval (See Hollender and Wolfe Nonparametric Statistical Methods 2nd Edition, Wiley, New York, 1999 p51-56). Calculations will be made using the STATEXACT™ (Cytel) statistical software package.

The Neuropathy Impairment Score test will provide a single score of neuropathic deficits and subset scores related to cranial nerve function, muscles weakness, reflexes, and sensation. The deficits will be scored by the examiner when compared to age and gender-related patients considering height, weight and physical fitness. Muscle weakness will be scored as 0 if normal, I if 25% weak, 2 if 50% weak, 3 if 75% weak, 3.25 if the muscle moves against gravity, 3.5 if there is movement when gravity is eliminated, 3.75 when there is a flicker without movement, and 4 if there is total paralysis. This will be applied to cranial nerves III, VI, VII, X and XII. Individual muscle groups tested for their strength include respiratory, neck flexion, shoulder abduction, elbow flexion, brachial radialis, elbow extension, wrist flexion and extension, finger flexion and spread, thumb abduction, hip flexion and extension, knee flexion and extension, ankle dorsiflexion, ankle plantar flexion toe extensor and flexors for a total of 24 items. Each group will be tested on the right and left sides.

The reflexes will be scored as 0=normal, I=decreased, 2=absent. Fiber-tendon reflexes will be examined on each side including biceps, triceps, brachial radialis, quadriceps, and triceps surae. For patients who are 60 years or older, ankle reflexes decrease will be graded as 0 and their absence will be graded as 1.

The sensory examination will be performed over the dorsum of the finger and great toe. Touch pressure will be measured by using a long cotton wool. Pinprick will be assessed with the use of straight pins. Vibration sensation is tested with a 165 Hz tuning fork, and joint position will be tested by moving the terminal phalanx of the index finger and great toe. The exam will be done on each extremity and the scoring will be 0=normal, I=decreased and 2=absent.

It is expected that rituximab or humanized 2H7 will exhibit patient improvement as defined above over a control (without such antibody), and therefore treat mononeuritis multiplex.

What is claimed is:

1. A method of treating polychondritis or mononeuritis multiplex in a mammal comprising administering to the mammal an effective amount of an antibody that binds CD20.

2. The method of claim 1 wherein the antibody is not conjugated with another molecule.

3. The method of claim 1 wherein the antibody is conjugated with another molecule.

4. The method of claim 3 wherein the other molecule is a cytotoxic agent.

5. The method of claim 4 wherein the cytotoxic agent is a radioactive compound.

6. The method of claim 5 wherein the cytotoxic agent comprises Y2B8 or ¹³¹I-B1.

7. The method of claim 1 wherein the antibody comprises rituximab.

8. The method of claim 1 wherein the antibody comprises a humanized 2H7.

9. The method of claim 1 comprising administering a dose of about 20 mg/m² to about 250 mg/m² of the antibody to the mammal.

10. The method of claim 9 wherein the dose is about 50 mg/m² to about 200 mg/m².

11. The method of claim 1 comprising administering an initial dose of the antibody followed by a subsequent dose, wherein the mg/m² dose of the antibody in the subsequent dose exceeds the mg/m² dose of the antibody in the initial dose.

12. The method of claim 1 wherein the mammal is human.

13. The method of claim 1 wherein the antibody is administered intravenously.

14. The method of claim 1 wherein the antibody is administered subcutaneously.

15. The method of claim 1 further comprising administering to the mammal an effective amount of an immunosuppressive agent, anti-pain agent, or a chemotherapeutic agent.

16. The method of claim 1 wherein polychondritis is treated.

17. The method of claim 16 further comprising administering to the mammal an effective amount of a nonsteroidal anti-inflammatory drug, steroid, methotrexate, cyclophosphamide, dapsone, azathioprine, penicillamine, or cyclosporine.

18. The method of claim 1 wherein mononeuritis multiplex is treated.

19. The method of claim 18 further comprising administering to the mammal an effective amount of an anti-pain agent, steroid, methotrexate, cyclophosphamide, plasma exchange, intravenous immunoglobulin, cyclosporine, or mycophenolate mofetil.

20. An article of manufacture comprising a container and a composition contained therein, wherein the composition comprises an antibody that binds CD20, and further comprising a package insert instructing the user of the composition to treat polychondritis or mononeuritis multiplex in a mammal.

21. The article of claim 20 further comprising a container comprising an agent other than the antibody for the treatment and further comprising instructions on treating the mammal with such agent.
